SEQUENCE LISTING (14 sequences)

SEQ ID NO: 1 - DNA, 18 bp, synthetic construct
  misc_feature 1..7: note = RNA
  source 1..18: mol_type = other DNA
  misc_feature 1: note = 5' Phosphorylated
  misc_feature >18: note = 3' C3 Spacer
  misc_feature 8..18: note = DNA
atcagcacttagcgtcag

SEQ ID NO: 2 - DNA, 24 bp, synthetic construct
  source 1..24: mol_type = other DNA
  misc_feature 8..9: note = internal biotin
  misc_feature 1: note = 5' Phosphorylated
tgacttgcgacgctaagtgctgat

SEQ ID NO: 3 - DNA, 32 bp, synthetic construct
  source 1..32: mol_type = other DNA
  misc_feature 1: note = 5' Phosphorylated
  misc_feature >32: note = 3' 2-MethoxyEthoxy G
  modified_base 28..32: mod_base = OTHER; note = Phosphorothioate bonds
aagaccaccagatcggaagagcgtcgtgtagg

SEQ ID NO: 4 - DNA, 32 bp, synthetic construct
  source 1..32: mol_type = other DNA
  misc_feature 1: note = 5' Phosphorylated
  misc_feature 15..16: note = internal biotin
tgacttgtcatgtctccgatctggtggtcttt

SEQ ID NO: 5 - DNA, 27 bp, synthetic construct
  source 1..27: mol_type = other DNA
  misc_feature >27: note = 3' C3 Spacer
tacacgacgctcttccgatctnnnnnn

SEQ ID NO: 6 - DNA, 21 bp, synthetic construct
  source 1..21: mol_type = other DNA
  misc_feature 1: note = 5' Phosphorylated
  misc_feature >21: note = 3' C3 Spacer
agatcggaagagcgtcgtgta

SEQ ID NO: 7 - DNA, 30 bp, synthetic construct
  source 1..30: mol_type = other DNA
  misc_feature 1: note = 5' Phosphorylated
aaacacccaagatcggaagagcgtcgtgta

SEQ ID NO: 8 - DNA, 33 bp, synthetic construct
  source 1..33: mol_type = other DNA
  misc_feature 1: note = 5' Phosphorylated
tgacttgtcatgtcttccgatcttgggtgtttt

SEQ ID NO: 9 - DNA, 48 bp, synthetic construct
  source 1..48: mol_type = other DNA
  misc_feature 1: note = 5' Phosphorylated
caagtcaagctagattccacgaagagttgtcacgtcagccgcagtatc

SEQ ID NO: 10 - DNA, 48 bp, synthetic construct
  source 1..48: mol_type = other DNA
  misc_feature 1: note = 5' Phosphorylated
tgacttggatactgcggctgacgtgacaactcttcgtggaatctagct

SEQ ID NO: 11 - DNA, 27 bp, synthetic construct
  source 1..27: mol_type = other DNA
agttgtcaccataataagatcggaaga

SEQ ID NO: 12 - DNA, 31 bp, synthetic construct
  source 1..31: mol_type = other DNA
cagacgtgtgctcttccgatcttattatggt

SEQ ID NO: 13 - DNA, 39 bp, synthetic construct
  misc_feature 1..12: note = RNA
  source 1..39: mol_type = other DNA
  misc_feature 1: note = 5' Phosphorylated
  misc_feature 13..39: note = DNA
atcagcacccggatgtagataggatggacttagcgtcag

SEQ ID NO: 14 - DNA, 37 bp, synthetic construct
  source 1..37: mol_type = other DNA
tgacttgctgacgctaagtccatcctatctacatccg

(19) United States
(12) Patent Application Publication
(10) Pub. No.: US 2023/0408504 A1
(43) Pub. Date: Dec. 21, 2023

(54) ANTIBODY BARCODED BEADS AND USES THEREOF

(71) Applicant: California Institute of Technology, Pasadena, CA (US)

(72) Inventors: Mitchell Guttman, Los Angeles, CA (US); Andrew A. Perez, Pasadena, CA (US); Mario R. Blanco, Los Angeles, CA (US); Isabel N. Goronzy, Pasadena, CA (US); James K. Guo, Pasadena, CA (US)

(21) Appl. No.: 18/167,362

(22) Filed: Feb. 10, 2023

Related U.S. Application Data
(60) Provisional application No. 63/309,436, filed on Feb. 11, 2022.

(51) Int. Cl. G01N 33/543

(52) U.S. Cl. CPC G01N 33/54353; G01N 33/54326; G01N 33/54346

(57) ABSTRACT

Disclosed herein include methods, compositions, and kits suitable for use in generating barcoded detection particles. Each barcoded detection particle can comprise a particle associated with an antigen-binding protein and a plurality of barcoding oligonucleotides. The plurality of barcoding oligonucleotides can comprise a first ligand. The particle can comprise a second ligand. The plurality of barcoding oligonucleotides can be associated with the particle via a multivalent binding agent comprising two or more binding moieties capable of binding the first ligand and/or the second ligand. There are provided, in some embodiments, methods for detecting interactions between nucleic acid molecules and proteins of interest. Methods for detecting interactions between ribonucleic acid molecules and RNA-binding proteins (RBPs) are also provided herein.

### RELATED APPLICATIONS

This application claims the benefit under 35 U.S.C. § 119(e) of U.S. Provisional Patent Application Ser. No. 63/309,436, filed Feb. 11, 2022, the content of this related application is incorporated herein by reference in its entirety for all purposes.

### STATEMENT REGARDING FEDERALLY SPONSORED R&D

This invention was made with government support under Grant No. DK127420 & DA053178 & HG012216 awarded by the National Institutes of Health. The government has certain rights in the invention.

### REFERENCE TO SEQUENCE LISTING

The present application is being filed along with a Sequence Listing in electronic format. The Sequence Listing is provided as a file entitled Revised-30KJ-365853-US, created Jun. 28, 2023, which is 21,670 bytes in size. The information in the electronic format of the Sequence Listing is incorporated herein by reference in its entirety.

### BACKGROUND

### Field

The present disclosure relates generally to the field of detecting macromolecule interactions, such as, for example, interactions between nucleic acid molecules and proteins of interest.

### Description of the Related Art

DNA is not randomly organized in the nucleus, but is instead structured around function. For decades, it has been known that DNA can change its compaction based on gene expression. For example, DNA is compacted into heterochromatin when genes are silenced, but is more accessible as open euchromatin when genes are activated. This compaction of DNA in the nucleus is thought to play an important role in gene regulation because it makes genes more or less accessible to regulatory proteins such as transcription factors, polymerase, and chromatin modifying proteins. However, it remains unclear how specific genes are positioned in the nucleus to achieve specific functions, such as regulating gene expression. Current genomic mapping methods include Chromatin Immunoprecipitation (ChIP) for protein-DNA interactions, Crosslinking and Immunoprecipitation (CLIP) for protein-RNA interactions, and RNA Antisense Purification (RAP). These approaches generate genome-wide maps of binding proteins on DNA or RNA or RNAs to other RNA or genomic DNA, respectively. While these methods have advanced our understanding of gene regulation, they map interactions of a single DNA or RNA binding protein or ncRNA at a time. Because of the importance of these interactions, major international research efforts have generated reference maps of genomic datasets—including mapping multiple proteins to DNA and RNA—across a defined set of human and mouse cell types or tissues. Comprehensive data sets have been produced by large consortia (e.g. ENCODE, RoadMap Epigenomics), involving many labs and large amounts of money. In spite of these major efforts, only a tiny fraction of the total number of predicted DNA and RNA binding proteins and ncRNAs have been successfully mapped, and of these most have been mapped in only a small number of cell types. Because these datasets are highly cell-type specific, it is critically important to enable the generation of comprehensive cell-type specific genomic maps for any cell-type of interest that would not be well represented in reference maps (e.g. patient samples, animal models, or perturbations). Yet, this goal has remained elusive for individual labs because of the time, cost, and labor required to generate such data with current approaches. There is a need for multiplexed methods of detecting macromolecule interactions, such as, for example, interactions between nucleic acid molecules and proteins of interest.

### SUMMARY

Disclosed herein include compositions. In some embodiments, the composition comprises: a plurality of barcoded detection particles. In some embodiments, each barcoded detection particle comprises a particle associated with an antigen-binding protein and a plurality of barcoding oligonucleotides. In some embodiments, the antigen-binding protein is associated with the particle via an immunoglobulin-binding moiety. In some embodiments, plurality of barcoding oligonucleotides comprise a first ligand. In some embodiments, the particle comprises a second ligand. In some embodiments, the plurality of barcoding oligonucleotides are associated with the particle via a multivalent binding agent comprising two or more binding moieties capable of binding the first ligand and/or the second ligand. Disclosed herein include compositions comprising a plurality of barcoded detection particles generated according to the methods disclosed herein.

Disclosed herein include methods for generating barcoded detection particles. In some embodiments, the method comprises: providing a plurality of barcoding oligonucleotides comprising a first ligand; providing a plurality of particles comprising an antigen-binding protein, a second ligand and an immunoglobulin-binding moiety; providing a plurality of multivalent binding agents comprising two or more binding moieties capable of binding the first ligand and/or the second ligand; and/or contacting the plurality of barcoding oligonucleotides, the plurality of particles, and the plurality of multivalent binding agents to generate a plurality of barcoded detection particles, wherein each of the barcoded detection particles comprises a particle associated with a plurality of barcoding oligonucleotides.

Disclosed herein include kits for the generation of barcoded detection particles. In some embodiments, the kit comprises: a plurality of barcoding oligonucleotides comprising a first ligand, or precursor(s) thereof a plurality of particles comprising an antigen-binding protein, a second ligand and an immunoglobulin-binding moiety, or precursor(s) thereof; and/or a plurality of multivalent binding agents comprising two or more binding moieties capable of binding the first ligand and/or the second ligand, or precursor(s) thereof.

Disclosed herein include compositions. In some embodiments, the composition comprises: a plurality of barcoded detection particles. In some embodiments, each barcoded detection particle comprises a particle associated with an antigen-binding protein and a plurality of barcoding oligonucleotides. In some embodiments, the antigen-binding protein is associated with the particle via an immunoglobulin-binding moiety. In some embodiments, the plurality of barcoding oligonucleotides are directly attached to the particle.

Disclosed herein include compositions. In some embodiments, the composition comprises: a plurality of barcoded detection particles. In some embodiments, each barcoded detection particle comprises a particle associated with an antigen-binding protein and a plurality of barcoding oligonucleotides. In some embodiments, the antigen-binding protein is associated with the particle via an immunoglobulin-binding moiety. In some embodiments, plurality of barcoding oligonucleotides are directly attached to the antigen-binding protein.

In some embodiments, the first ligand and/or the second ligand are the same. In some embodiments, the first ligand and/or the second ligand are different. In some embodiments, the two or more binding moieties are the same. In some embodiments, at least one of the two or more binding moieties are different. In some embodiments, the first ligand comprises biotin; the second ligand comprises biotin; the plurality of multivalent binding agents comprise streptavidin; the particle is a Dynabead; and/or the immunoglobulin-binding moiety comprises Protein G. In some embodiments, providing a plurality of barcoding oligonucleotides comprising a first ligand comprises: attaching the first ligand to each of the barcoding oligonucleotides (e.g., via click chemistry).

In some embodiments, providing a plurality of particles comprising an antigen-binding protein, a second ligand and an immunoglobulin-binding moiety comprises: attaching the immunoglobulin-binding moiety to the particles (e.g., via click chemistry); attaching the second ligand to the particles (e.g., via click chemistry); and attaching the antigen-binding protein to the particles via the interaction between the antigen-binding protein and the immunoglobulin-binding moiety. In some embodiments, the contacting step comprises: contacting the plurality of multivalent binding agents and the plurality of particles to form first intermediate complexes, and contacting said first intermediate complexes with the plurality of barcoding oligonucleotides to generate the plurality of barcoded detection particles; or contacting the plurality of barcoding oligonucleotides and the plurality of multivalent binding agents to form second intermediate complexes, and contacting said second intermediate complexes with the plurality of plurality of particles to generate the plurality of barcoded detection particles.

In some embodiments, the first ligand and at least one of the two or more binding moieties are a specific binding pair, wherein the specific binding pair comprises a first member of a specific binding pair and a second member of a specific binding pair that bind one another with: (i) high affinity, high avidity, and/or high specificity, or (ii) low affinity, low avidity, and/or low specificity. In some embodiments, the second ligand and at least one of the two or more binding moieties are a specific binding pair, wherein the specific binding pair comprises a first member of a specific binding pair and a second member of a specific binding pair that bind one another with: (i) high affinity, high avidity, and/or high specificity, or (ii) low affinity, low avidity, and/or low specificity.

In some embodiments, the binding between the first and second member of the specific binding pair occurs via covalent bonding. In some embodiments, the binding between the first and second member of the specific binding pair occurs via non-covalent interactions. In some embodiments, the non-covalent interactions comprise one or more of ionic bonding, hydrophobic interactions, van der Waals forces, and hydrogen bonding. In some embodiments, the binding between the first and the second member of the specific binding pair has a dissociation constant K_{d} between about 10⁻¹⁰ to about 10⁻¹⁵ mol/L.

In some embodiments, the first and the second member of the specific binding pair is each selected from the group comprising an antibody or an antigen-binding portion thereof and an antigen, an biotin moiety and an avidin moiety, a dinitrophenol (DNP) and an anti-DNP antibody, a digoxin and an anti-digoxin antibody, a digoxigenin and an anti-digoxigenin antibody, a hapten and an anti-hapten, a polysaccharide and a polysaccharide binding moiety, a lectin and a receptor, a ligand and a receptor, a fluorescein and an anti-fluorescein antibody, complementary nucleic acids, derivatives therefore, and fragments thereof. In some embodiments, the multivalent binding agent, the first ligand, the second ligand, and/or at least one of the two or more binding moieties is a biotin moiety and/or an avidin moiety. In some embodiments, the biotin moiety is selected from the group comprising biotin (cis-hexahydro-2-oxo-1H-thieno[3,4]imidazole-4-pentanoic acid) and derivatives or analogs thereof that can specifically bind to an avidin moiety. In some embodiments, the biotin moiety is selected from the group comprising biotin-e-N-lysine, biocytin hydrazide, amino or sulfhydryl derivatives of 2-iminobiotin and biotinyl-6-aminocaproic acid-N-hydroxysuccinimide ester, sulfosuccinimideiminobiotin, biotinbromoacetylhy dr azide, p-diazobenzoyl biocytin, 3-(N-maleimidopropionyl)biocytin, oxybiotin, 2′-iminobiotin, diaminobiotin, biotin sulfoxide, biocytin, 9-methylbiotin, biotin methyl ester (MEBio), desthiobiotin (DEBio), e-N-Biotinyl-L-lysine, diaminobiotin (DABio), biotin sulfone, 2′-thiobiotin and N3′-ethyl biotin, and derivatives thereof. In some embodiments, the avidin moiety comprises native egg-white glycoprotein avidin, or any derivatives, analogs, fragments and other non-native forms thereof that can specifically bind to a biotin moiety. In some embodiments, the avidin moiety comprises an N-acyl avidin. In some embodiments, the N-acyl avidin comprises N-acetyl avidin, N-phthalyl avidin, N-succinyl avidin, and derivatives thereof. In some embodiments, the avidin moiety comprises streptavidin, nitrostreptavidin, ExtrAvidin, Captavidin, Neutravidin, Neutralite Avidin, and derivatives thereof.

In some embodiments, the immunoglobulin-binding moiety comprises Protein L, Protein G, Protein A, Protein A/G, or a combination thereof. In some embodiments, the antigen binding protein comprises an antibody, an antibody fragment, an scFv, a Fv, a Fab, a (Fab′)2, a single domain antibody (SDAB), a VH or VL domain, a camelid VHH domain, a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. In some embodiments, the antigen binding protein is capable of specifically binding a protein of interest. In some embodiments, the antigen binding protein is not conjugated to an oligonucleotide. In some embodiments, the protein of interest is: a histone modification (e.g., H2AZK4/K7Ac, H2BK12Ac, H2BK15Ac, H2BK20Ac, H3K14Ac, H3K18Ac, H3K9Ac, H3K27Ac, H3K36Ac, H3K56Ac, H3K9/K14Ac, H4K5Ac, H4K12Ac, H4K16Ac, H3 Ser10p, H3Thr3p, H2AK119ub, H2AK120ub, H3K4me1, H3K79me1, H3K9me1, H3K27me2, H3K4me2, H3K79me2, H3K9me2, H3K9me2/me3, H3K4me3, H3K36me3, H3K36me1, H3K36me2, H3K79me3, H3K9me3, H4K20me3, H3R8me2, H3R3me2, H3R18me2, or any combination thereof); and/or chromatin-associated proteins, such as transcription factors, chromatin regulators or polymerases, and their modified forms (e.g., AEBP2, ATF2, BCL6, Beta Catenin, CBFβ, CDK8 NELFb, CREB, CTCF, DNMT3A, DNMT3B, E2F1, E2F4, EGR1, ELK1, ELL, FoxP1, HIF 1, INT S9, KLF5, LAP1α, LAP1β, MAX, MAZ, MBD2, MBD3, MITF, MNT, MeCP2, NRF1, Nanog, Pou5f1, RAD21, RBPJ, RFX1, RNF20, RING1, SP1, SPT16, SRF, Suz12, Sox2, TAF1, TBP, TCF4, TET1, TET2, TH1L, USF2, UTX, YY1, ZNF24, ZNF687, cFos, cFos-pSer32, cJun, cJun-pSer63, cJun-pSer73, P53, P53-pSer15, POLR1A, POLR2A, POLR2A-pSer2, POLR2A-pSer5, POLR2A-pSer2/5, POLR3A, POLR2A-pThr4, POLR3D, POLR3E, ASH2, BAF57, BRD3, BRD4, BRG1, CBP, CLOCK, ESET, EZH2, G9a, HDAC1, HDAC2, HDAC3, HDAC5, HDAC6, HP1α, HP1β, JARID1A, JARID1B, JARID2, JMJD2A, LSD1, MLL, MTA1, MTA2, Menin, NFRkB, PCAF, PHC1, PHF8, RBBP5, RING1B, SAP30, SETD1A, SETD2, SIN3A, SIRT6, SPT4, SPT6, SRC3, SSRP1, WDR5, ZMYND11, or any combination thereof). In some embodiments, the protein of interest is: a transcriptional regulator (e.g., ILF3, SAF-B, TAF15, EWSR1, WDR43, TARDBP, FUBP3, SSB, SLBP, ELAVL1, SHARP, FUS, RBM15, SAF-A, TIAL1, LBR, or any combination thereof); an RNA processing factor (e.g., CPSF6, hnRNPC, hnRNPL, KHSRP, LIN28B, PTBP1, QKI, RBM5, U2AF1, hnRNPM, NOLC1, TRA2A, BUD13, RBFOX2, DROSHA, DGCR8, LSM11, SMNDC1, hnRNPA1, DDX52, DDX55, AQR, SRSF9, or any combination thereof); and/or a translational regulator (e.g., PCBP1, PCBP2, IGF2BP1, IGF2BP2, IGF2BP3, RP S3, UPF1, LARP1, FASTKD2, LARP4, LARP7, DDX6, PUM1, DHX30, or any combination thereof).

In some embodiments, the particle is or comprises a bead, a gold bead, polysaccharide, poly(acrylate), polystyrene, poly(acrylamide), polyol, agarose, agar, cellulose, dextran, starch, heparin, glycogen, amylopectin, mannan, inulin, nitrocellulose, diazocellulose, polyvinylchloride, polypropylene, polyethylene, nylon, a latex bead, a conducting metal, a nonconducting metal, glass, a magnetic bead, a paramagnetic bead, a superparamagnetic bead, or any combination thereof. In some embodiments, each particle is associated with at least about 2, about 3, about 4, about 5, about 6, about 7, about 8, about 9, about 10, about 11, about 12, about 14, about 16, about 18, about 20, about 30, about 40, about 50, about 60, about 70, about 80, or about 100, barcoding oligonucleotides. In some embodiments, each barcoding oligonucleotide comprises a capture barcode, wherein the capture barcode is a unique sequence specific to the antigen-binding protein associated with the particle to which the barcoding oligonucleotide is associated with. In some embodiments, the first ligand is situated: (i) on the 3′ end of the barcoding oligonucleotide, (ii) on the 5′ end of the barcoding oligonucleotide, and/or (iii) internally between the 5′ end and the 3′ end of the barcoding oligonucleotide. In some embodiments, the 5′ end of the barcoding oligonucleotide comprises a modified phosphate group and/or a 5′ overhang capable of ligation to the 5′ overhang of a combinatorial barcode unit. In some embodiments, the barcoding oligonucleotide comprises a unique molecular identifier (UMI), which can be about 8 nucleotides in length. In some embodiments, the barcoding oligonucleotide comprises a universal library sequence. In some embodiments, the universal library sequence comprises a sequence complementary to at least a portion of a sequencing primer, such as, for example, an Illumina-compatible sequencing primer sequence (e.g., a Read 1 sequencing primer or a Read 2 sequencing primer). In some embodiments, the barcoding oligonucleotide further comprises a spacer sequence (e.g., a 3′ spacer sequence).

Disclosed herein include compositions comprising a pool of barcoded detection particles disclosed herein. In some embodiments, the pool of barcoded detection particles comprises: two or more barcoded detection particles that differ from each other with respect to the antigen-binding protein associated with the particle. In some embodiments, each barcoding oligonucleotide comprises a capture barcode, wherein one or more of the plurality of barcoded detection particles comprises two or more barcoding oligonucleotides having distinct capture barcodes, and wherein each barcoded detection particle of the plurality of barcoded detection particles has a unique set of one or more capture barcode(s) associated therewith specific to the antigen-binding protein associated with the particle to which the barcoding oligonucleotide is associated with. The pool of barcoded detection particles can comprise: at least about 5, about 10, about 20, about 30, about 50, about 75, about 100, about 150, about 200, about 250, about 300, about 350, about 400, about 450, about 500, about 550, about 600, about 650, about 700, about 750, about 800, about 850, about 900, about 950, about 1000, about 2000, about 3000, about 4000, about 5000, about 6000, about 7000, about 8000, about 9000, or about 10000, barcoded detection particles that differ from each other with respect to the antigen-binding protein and barcoding oligonucleotide associated with the particle.

Disclosed herein include methods for detecting interactions between nucleic acid molecules and proteins of interest. In some embodiments, the method comprises: lysing a sample comprising a plurality of cells to generate a cell lysate, wherein the cells comprise nucleic acid molecules suspected of being associated with proteins of interest. The method can comprise providing a pool of barcoded detection particles provided herein. The method can comprise: contacting the cell lysate, or a product thereof, with the pool of barcoded detection particles disclosed herein to form a plurality of detection complexes, wherein each of the plurality of detection complexes comprises: a barcoded detection particle; a captured protein of interest; and captured nucleic acid molecule(s) associated with the captured protein of interest. The method can comprise: performing one or more (e.g., two or more iterations) of split-and-pool barcoding, wherein each iteration comprises: (i) randomly distributing the plurality of detection complexes into a plurality of partitions; (ii) in the plurality of partitions, combinatorially barcoding captured nucleic acid molecules and barcoding oligonucleotides, or products thereof, with a combinatorial barcode unit, wherein within each partition, the captured nucleic acid molecules and barcoding oligonucleotides are barcoded with the same combinatorial barcode unit, wherein captured nucleic acid molecules and barcoding oligonucleotides of different partitions receive different combinatorial barcode units from each other, and wherein captured nucleic acid molecules and barcoding oligonucleotides of the same detection complex will assort together in a partition of the plurality of partitions; and (iii) pooling the detection complexes from the plurality of partitions, wherein, after said two or more iterations of split-and-pool barcoding, each combinatorially barcoded captured nucleic acid molecule and each combinatorially barcoded barcoding oligonucleotide comprises a combinatorial barcode comprising two or more combinatorial barcode units, wherein each combinatorial barcode unit corresponds to an iteration of split-and-pool-barcoding. The method can comprise: obtaining sequence information of the combinatorially barcoded captured nucleic acid molecules and the combinatorially barcoded barcoding oligonucleotides, or products thereof. The method can comprise: detecting interactions between captured nucleic acid molecules and proteins of interest based on the sequence information.

The method can further comprise: adding a crosslinking agent (e.g., formaldehyde) to the plurality of cells prior to the lysis step; or adding a crosslinking agent to the cell lysate. In some embodiments, the method further comprises: isolation of the nuclei of the plurality of cells. In some embodiments, the nuclei of the plurality of cells are obtained via centrifugation of the cell lysate. In some embodiments, the method further comprises: fragmentation of the chromatin of the plurality of cells. In some embodiments, fragmentation comprises enzymatic chromatin fragmentation and/or sonication of the nuclear pellet. In some embodiments, said fragmentation generates chromatin fragments of about 150 bp to about 700 bp, and can have an average size of about 350 bp. In some embodiments, the method further comprises: processing at least one end of the captured nucleic acid molecule(s) to enable ligation of said captured nucleic acid molecule(s) to a ligation adaptor molecule, wherein said processing comprises blunt-ending, phosphorylation, and/or dA-tailing. In some embodiments, the method further comprises: ligating a ligation adaptor molecule (e.g., an DNA Phosphate Modified (DPM) tag) to the captured nucleic acid molecule(s).

In some embodiments, a probability of the interaction between the nucleic acid molecule and the protein of interest as being bona fide is proportional to the number of iterations of split-and-pool barcoding. In some embodiments, performing two or more iterations of split-and-pool barcoding comprises performing n*2 iterations of split-and-pool barcoding, wherein n is an integer greater than zero, wherein the combinatorial barcode unit is an Odd tag or an Even tag, and wherein each set of two iterations comprises barcoding with an Odd tag followed by barcoding with an Even tag. In some embodiments, performing two or more iterations of split-and-pool barcoding comprises performing n iterations of split-and-pool barcoding, wherein n is an integer greater than one. In some embodiments, n is at least about 2, about 3, about 4, about 5, about 6, about 7, about 8, about 9, about about 11, about 12, about 14, about 16, about 18, about 20, about 30, about 40, about 50, about about 70, about 80, or about 100. In some embodiments, each combinatorial barcode unit comprises at least one 5′ overhang, and wherein said 5′ overhang is capable of ligating to a 5′ overhang of one or more of a ligation adaptor molecule, a combinatorial barcode unit, or a terminal tag. In some embodiments, each combinatorial barcode unit comprises a modified 5′ phosphate group. In some embodiments, the combinatorial barcoding step comprises: annealing the 5′ overhang of a barcoding oligonucleotide, a ligation adaptor molecule, or a combinatorial barcode unit, to the 5′ overhang of a combinatorial barcode unit; and ligating the annealed molecules. In some embodiments, the method further comprises, following the two or more iterations of split-and-pool barcoding: annealing a terminal tag to each captured nucleic acid molecule and each barcoding oligonucleotide; and ligating said annealed molecules. In some embodiments, the barcoding oligonucleotide and/or terminal tag further comprises a spacer sequence, such as, for example, a 3′ spacer sequence that allows the combinatorial barcode unit to only ligate to the 5′ end of each single-stranded DNA sequence and prevents formation of hairpins during library amplification. In some embodiments, the method further comprises: reversing crosslinking to elute the combinatorially barcoded captured nucleic acid molecules and the combinatorially barcoded barcoding oligonucleotides from the particles.

In some embodiments, obtaining sequence information comprises amplifying the combinatorially barcoded captured nucleic acid molecules and the combinatorially barcoded barcoding oligonucleotides. In some embodiments, obtaining sequence information comprises: obtaining sequencing data comprising a plurality of sequencing reads of the combinatorially barcoded captured nucleic acid molecules and the combinatorially barcoded barcoding oligonucleotides, or products thereof. In some embodiments, each of the plurality of sequencing reads of the combinatorially barcoded captured nucleic acid molecules, or products thereof, comprise: a combinatorial barcode sequence, and a captured nucleic acid molecule sequence. In some embodiments, each of the plurality of sequencing reads of the combinatorially barcoded barcoding oligonucleotides, or products thereof, comprise: a combinatorial barcode sequence, and a capture barcode sequence, wherein the capture barcode is a unique sequence specific to the antigen-binding protein associated with the particle to which the barcoding oligonucleotide is associated with.

In some embodiments, detecting interactions comprises: for each unique combinatorial barcode sequence, which indicates a single detection complex of the plurality of detection complexes, identifying the captured nucleic acid molecule sequence and capture barcode sequence of sequencing reads sharing a combinatorial barcode sequence. In some embodiments, detecting interactions comprises: for each unique capture barcode sequence, which indicates a captured protein of interest, identifying the captured nucleic acid molecule sequence of sequencing reads sharing a capture barcode sequence. In some embodiments, the method further comprises determining the binding site of a captured protein of interest on associated captured nucleic acid molecule sequence(s). In some embodiments, the method further comprises aligning captured nucleic acid molecule sequence(s) to a reference genome. In some embodiments, the nucleic acid molecules comprise deoxyribonucleic acid molecules and/or ribonucleic acid molecules. In some embodiments, the nucleic acid molecules are selected from the group comprising double-stranded DNA, single-stranded DNA, microRNA (miRNA), messenger RNA (mRNA), long non-coding RNA (lncRNA), ribosomal RNA (rRNA), transfer RNA (tRNA), small nuclear RNA (snRNA), small nucleolar RNA (snoRNA), Piwi-interacting RNA (piRNA), interfering RNA (siRNA), antisense RNA (aRNA), transfer messenger RNA (tmRNA), tRNA-derived small RNA (tsRNA), rDNA-derived small RNA (srRNA), ribozyme, viral RNA, single-stranded RNA, double-stranded RNA, or any combination thereof. In some embodiments, detecting interactions between nucleic acid molecules and proteins of interest comprises detecting interactions between nucleic acid molecules and at least about 2, about 4, about 6, about 8, about 10, about 12, about 14, about 16, about 18, about 20, about 30, about 40, about 50, about 60, about 70, about 80, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 2000, about 3000, about 4000, about 5000, about 6000, about 7000, about 8000, about 9000, or about 10000, different proteins of interest.

Disclosed herein include methods for detecting interactions between ribonucleic acid molecules and RNA-binding proteins (RBPs). In some embodiments, the method comprises: lysing a sample comprising a plurality of cells to generate a cell lysate, wherein the cells comprise ribonucleic acid molecules suspected of being associated with RBPs. The method can comprise providing a pool of barcoded detection particles provided herein. The method can comprise: contacting the cell lysate, or a product thereof, with the pool of barcoded detection particles disclosed herein to form a plurality of detection complexes, wherein each of the plurality of detection complexes comprises: a barcoded detection particle; a captured RBP; and captured ribonucleic acid molecule(s) associated with the captured RBP. The method can comprise: converting the captured ribonucleic acid molecule(s) to complementary DNA (cDNA) molecules. The method can comprise: performing one or more (e.g., two or more iterations) of split-and-pool barcoding, wherein each iteration comprises: (i) randomly distributing the plurality of detection complexes into a plurality of partitions; (ii) in the plurality of partitions, combinatorially barcoding cDNA molecules and barcoding oligonucleotides, or products thereof, with a combinatorial barcode unit, wherein within each partition, the cDNA molecules and barcoding oligonucleotides are barcoded with the same combinatorial barcode unit, wherein cDNA molecules and barcoding oligonucleotides of different partitions receive different combinatorial barcode units from each other, and wherein cDNA molecules and barcoding oligonucleotides of the same detection complex will assort together in a partition of the plurality of partitions; and (iii) pooling the detection complexes from the plurality of partitions, wherein, after said two or more iterations of split-and-pool barcoding, each combinatorially barcoded cDNA molecule and each combinatorially barcoded barcoding oligonucleotide comprises a combinatorial barcode comprising two or more combinatorial barcode units, wherein each combinatorial barcode unit corresponds to an iteration of split-and-pool-barcoding. The method can comprise: obtaining sequence information of the combinatorially barcoded cDNA molecules and the combinatorially barcoded barcoding oligonucleotides, or products thereof. The method can comprise: detecting interactions between captured ribonucleic acid molecules and RBPs based on the sequence information.

In some embodiments, the method further comprises: crosslinking interacting ribonucleic acids and RBPs (e.g., via UV crosslinking and/or via a crosslinking agent). Said UV crosslinking can comprise about 0.01 J cm⁻² to about 25 J cm⁻² of UV at about 100 nm to about 400 nm, such as, for example, about 0.25 J cm⁻² (UV 2.5k) of UV at about 254 nm. The crosslinking step can comprise contacting (e.g., contacting samples, cells, detection complexes) with 4-Thiouridine (4SU) and/or 6-thioguanosine (6SG). In some embodiments, the method further comprises DNase digestion and/or sonication of the cell lysate. In some embodiments, the method further comprises: partial fragmentation of the ribonucleic acids of the plurality of cells. The partial fragmentation can be enzyme-mediated (e.g., via RNase If). Said partial fragmentation can generate ribonucleic acids of about 300 bp to about 400 bp in length. In some embodiments, the method further comprises: processing the 3′ ends of captured ribonucleic acid molecules to enable ligation of said captured ribonucleic acid molecules to a ligation adaptor molecule. Said processing can comprises end repair (e.g., using T4 Polynucleotide Kinase). End repair can comprise processing said captured ribonucleic acid molecules to have 3′ OH groups compatible for ligation. In some embodiments, the method further comprises: ligating a ligation adaptor molecule (e.g., an RNA Phosphate Modified (RPM) tag) to the captured ribonucleic acid molecules. Ligating can comprise use of RNA Ligase I. In some embodiments, converting the captured ribonucleic acid molecule(s) to complementary DNA (cDNA) molecules is performed after ligation of the ligation adaptor molecule using a reverse transcription primer having a 5′ overhang capable of ligation to the 5′ overhang of a combinatorial barcode unit.

In some embodiments, a probability of the interaction between the ribonucleic acid molecule and the RBP as being bona fide is proportional to the number of iterations of split-and-pool barcoding. In some embodiments, performing two or more iterations of split-and-pool barcoding comprises performing n*2 iterations of split-and-pool barcoding, wherein n is an integer greater than zero, wherein the combinatorial barcode unit is an Odd tag or an Even tag, and wherein each set of two iterations comprises barcoding with an Odd tag followed by barcoding with an Even tag. In some embodiments, performing two or more iterations of split-and-pool barcoding comprises performing n iterations of split-and-pool barcoding, wherein n is an integer greater than one. In some embodiments, n is at least about 2, about 3, about 4, about 5, about 6, about 7, about 8, about 9, about about 11, about 12, about 14, about 16, about 18, about 20, about 30, about 40, about 50, about about 70, about 80, or about 100. In some embodiments, each combinatorial barcode unit comprises at least one 5′ overhang, and wherein said 5′ overhang is capable of ligating to a 5′ overhang of one or more of a reverse transcription primer, a combinatorial barcode unit, or a terminal tag. In some embodiments, each combinatorial barcode unit comprises a modified 5′ phosphate group. In some embodiments, the combinatorial barcoding step comprises: annealing the 5′ overhang of a barcoding oligonucleotide, a reverse transcription primer, or a combinatorial barcode unit, to the 5′ overhang of a combinatorial barcode unit; and ligating the annealed molecules. In some embodiments, the method further comprises, following the two or more iterations of split-and-pool barcoding: annealing a terminal tag to each cDNA molecule and each barcoding oligonucleotide; and ligating said annealed molecules. In some embodiments, the barcoding oligonucleotide and/or terminal tag further comprises a spacer sequence, such as, for example, a 3′ spacer sequence that allows the combinatorial barcode unit to only ligate to the 5′ end of each single-stranded DNA sequence and prevents formation of hairpins during library amplification. In some embodiments, the method further comprises: reversing crosslinking to elute the combinatorially barcoded cDNA molecules and the combinatorially barcoded barcoding oligonucleotides from the particles.

In some embodiments, obtaining sequence information comprises amplifying the combinatorially barcoded cDNA molecules and the combinatorially barcoded barcoding oligonucleotides. In some embodiments, the amplifying step comprises: ligating a chimeric ssDNA-dsDNA adaptor to the 3′ ends of the combinatorially barcoded cDNA molecules via a splint ligation reaction. In some embodiments, said chimeric ssDNA-dsDNA adaptor comprises a random sequence that anneals to the 3′ end of the cDNA. In some embodiments, obtaining sequence information comprises: obtaining sequencing data comprising a plurality of sequencing reads of the combinatorially barcoded cDNA molecules and the combinatorially barcoded barcoding oligonucleotides, or products thereof. In some embodiments, each of the plurality of sequencing reads of the combinatorially barcoded cDNA molecules, or products thereof, comprise: a combinatorial barcode sequence, and a captured cDNA molecule sequence. In some embodiments, each of the plurality of sequencing reads of the combinatorially barcoded barcoding oligonucleotides, or products thereof, comprise: a combinatorial barcode sequence, and a capture barcode sequence, wherein the capture barcode is a unique sequence specific to the antigen-binding protein associated with the particle to which the barcoding oligonucleotide is associated with.

In some embodiments, detecting interactions comprises: for each unique combinatorial barcode sequence, which indicates a single detection complex of the plurality of detection complexes, identifying the captured cDNA molecule sequence and capture barcode sequence of sequencing reads sharing a combinatorial barcode sequence. In some embodiments, detecting interactions comprises: for each unique capture barcode sequence, which indicates a captured RBP, identifying the captured cDNA molecule sequence of sequencing reads sharing a capture barcode sequence. In some embodiments, the method further comprises determining the binding site of a captured RBP on associated captured ribonucleic acid molecule sequence(s). In some embodiments, the method further comprises aligning captured cDNA molecule sequence(s) to a reference genome. In some embodiments, the ribonucleic acid molecules are selected from the group comprising microRNA (miRNA), messenger RNA (mRNA), long non-coding RNA (lncRNA), ribosomal RNA (rRNA), transfer RNA (tRNA), small nuclear RNA (snRNA), small nucleolar RNA (snoRNA), Piwi-interacting RNA (piRNA), interfering RNA (siRNA), antisense RNA (aRNA), transfer messenger RNA (tmRNA), tRNA-derived small RNA (tsRNA), rDNA-derived small RNA (srRNA), ribozyme, viral RNA, single-stranded RNA, double-stranded RNA, or any combination thereof. In some embodiments, detecting interactions between ribonucleic acid molecules and RBPs comprises detecting interactions between ribonucleic acid molecules and at least about 2, about 4, about 6, about 8, about 10, about 12, about 14, about 16, about 18, about 20, about 30, about 40, about about 60, about 70, about 80, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 2000, about 3000, about 4000, about 5000, about 6000, about 7000, about 8000, about 9000, or about 10000, different RBPs.

In some embodiments, the contacting step comprises immunoprecipitation. In some embodiments, the plurality of partitions are in fluid isolation from each other and/or comprise wells, microwells, tubes, vials, microcapsules, droplets, or any combination thereof. In some embodiments, the plurality of partitions comprise at least about 6, about 8, about 10, about 12, about 14, about 16, about 18, about 20, about 22, about 24, about 26, about 28, about 30, about 32, about 34, about 36, about 38, about 40, about 42, about 44, about 46, about 48, about 50, about 52, about 54, about 56, about 58, about 60, about 62, about 64, about 66, about 68, about 70, about 72, about 74, about 76, about 78, about 80, about 82, about 84, about 86, about 88, about 90, about 92, about 94, about 96, about 98, or about 100, different partitions comprising different combinatorial barcode units from each other.

In some embodiments, the sample is obtained from a subject, such as, for example, an organ of the subject, a tissue of the subject, or a bodily fluid of the subject. In some embodiments, the plurality of cells comprise immortalized cells and/or primary cells. In some embodiments, the plurality of cells comprise a eukaryotic cell. In some embodiments, the eukaryotic cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof. In some embodiments, the plurality of cells comprise cells of human origin or cells of non-human origin, such as, for example, mouse cells, rat cells, rabbit cells, pig cells, bovine cells, primate cells, non-mammalian cells, fish cells, insect cells, mold cells, dictostelium cells, worm cells, or drosophila cells.

In some embodiments, lysing a sample comprising a plurality of cells comprises lysing a plurality of samples each comprising a plurality of cells. In some embodiments, the plurality of samples differ with respect to cell type. In some embodiments, detection complexes of the same sample are labeled with a nucleic acid comprising the same unique sample identifier sequence, wherein detection complexes of different samples differ with respect to the unique sample identifier sequence added during said labeling, and wherein the method comprises pooling the detection complexes of different samples after said labeling step and prior to performing two or more iterations of split-and-pool barcoding. In some embodiments, the ligation adaptor molecule comprises a unique sample identifier sequence. In some embodiments, each of the sequencing reads of the combinatorially barcoded captured nucleic acid molecule, the combinatorially barcoded cDNA molecules and/or the combinatorially barcoded barcoding oligonucleotides, or products thereof, comprise a unique sample identifier sequence. In some embodiments, the method comprises identifying the sample origin of detection complexes based on the unique sample identifier sequence of one or more sequencing reads originating from said detection complexes.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 depicts a non-limiting exemplary schematic of the ChIP-DIP workflow.

FIGS. 2A-2E depict data related to a small proof-of-concept panel (CTCF, POLR2A, H3K4me3, H3K27me3, and IgG) ChIP-DIP experiments, including the assigning of cluster barcodes to target proteins using SA-Oligo reads (FIG. 2A), separated tracks of individual target proteins (FIG. 2B), track comparison with ENCODE (FIG. 2C and FIG. 2D), and track correlations with ENCODE (FIG. 2E).

FIGS. 3A-3H depict data related to a set of larger proof-of-concept ChIP-DIP experiments interrogating over 50 targets (a Histone and ABCAM Panel, a Transcription Factor and Chromatin Regulator Panel and an All Class of Targets Panel), including the distribution of beads assigned to each target (Histone and ABCAM Panel and Transcription Factor and Chromatin Regulator Panel; FIG. 3A), Pearson correlation matrices comparing track coverage with ENCODE (Histone and ABCAM Panel, FIG. 3B; Transcription Factor and Chromatin Regulator Panel, FIG. 3C), a comparison of the performance of multiple antibodies targeting the same protein (Transcription Factor and Chromatin Regulator Panel, FIG. 3D), visualization of multiple targets responsible for gene silencing at the Hox Gene Cluster (Transcription Factor and Chromatin Regulator Panel, FIG. 3E), visualization of various phosphorylation states of POLR2A (Transcription Factor and Chromatin Regulator Panel, FIG. 3F), visualization of various methylation states of H3K79 (Histone and ABCAM Panel, FIG. 3G), and a Pearson correlation matrix comparing track coverage with ENCODE over a diverse set of targets (All Class of Targets Panel, FIG. 311).

FIG. 4 depicts a non-limiting exemplary schematic related to the challenge of elucidating gene regulation via mapping of proteins on chromatin.

FIG. 5 depicts a non-limiting exemplary ChIP-DIP workflow.

FIGS. 6A-6D depict data related to ChIP-DIP experiments interrogating a panel of model proteins (CTCF, POLR2A, H3K4me3, H3K27me3, and IgG), including signal (log 10) comparisons with ENCODE (FIG. 6A), peak-centered coverage comparison with ENCODE (FIG. 6B), sensitivity and specificity of target detection relative to ENCODE (FIG. 6C), and ChIP-DIP reproducibility (FIG. 6D).

FIGS. 7A-7E depict data related to ChIP-DIP experiments demonstrating the diversity of proteins capable of being handled by the method, including many histone proteins (FIG. 7A), many chromatin regulators (FIG. 7B and FIG. 7C), and many transcription factors (FIG. 7D), as well as data demonstrating that ChIP-DIP data can be used to accurately calls motifs (FIG. 7E).

FIGS. 8A-8D depict data related to ChIP-DIP cell type accessibility, including data showing operation of ChIP-DIP with minimal cell numbers (FIG. 8A and FIG. 8B), data showing genome wide concordance as input decreases (FIG. 8C), and data showing Pearson correlations with active or repressive histone modifications for different antibody signals (FIG. 8D).

FIG. 9 depicts a non-limiting exemplary workflow for Split Pool Identification of RBPs (SPIDR).

FIGS. 10A-10F depict data related to proof-of-concept SPIDR experiments, including mapped RNA reads for XIST RBPs SHARP, hnRNPK, PTBP1, and SAF-A (pre-deconvolution, FIG. 10A; post-deconvolution, FIG. 10B), mapped RNA reads for splicing proteins FUS and KHSRP (FIG. 10C), mapped RNA reads for translation proteins hnRNPK and PCBP2 (FIG. 10D), mapped RNA reads for translation protein LARP1 (FIG. 10E), and a comparison of RBP motifs generated by SPIDR, ENCODE RNBS, and ENCODE eCLIP (FIG. 10F).

FIG. 11A is a non-limiting exemplary schematic showing the molecular biology steps performed for ligating DNA molecules in a cell lysate with a series of unique nucleotide tags in order to barcode molecules in the same complex with the same barcode, according to embodiments disclosed herein. As a first step, the DNA is end-repaired and dA-tailed, and then a complementary dT overhang DNA Phosphate modified (DPM) adaptor (shown in red) is ligated to both ends of the DNA molecule. After the DPM adaptor is ligated, all molecules can be pooled and redistributed in a multi-well (e.g., 96-well) format and can be then tagged with a first set of “Odd” nucleotide tags (shown in green) which can be capable of ligating to the preceding DPM nucleotide tag (shown in red) on both ends of each DNA molecule. After the Odd nucleotide tag is ligated, all molecules can be pooled and redistributed in a (e.g., 96-well) format and can be then tagged with a first set of “Even” nucleotide tags (shown in blue) which can be capable of ligating to the preceding Odd nucleotide tag on both ends of each DNA molecule. After the Even nucleotide tags have been ligated, all molecules can be pooled and redistributed in a multi-well format and in the schematic shown, can be tagged with a Terminal tag sequence capable of ligating to the preceding Even nucleotide tag.

FIG. 11B is a non-limiting exemplary schematic of DNA Phosphate Modified (DPM) adaptor tags, according to embodiments disclosed herein. The DPM Adaptor tags can be double stranded (ds) DNA in which the 5′ end of the molecule has a modified phosphate group (5′ Phos) that allows for the ligation between the DPM adaptor tag and the target DNA molecules as well as the subsequent nucleotide tag (e.g., the first Odd nucleotide tag). The highlighted regions on the DPM have the following functions: the yellow T overhang is a mini-sticky-end that ligates to the end-repaired target DNA molecules; the pink region may serve as an optionally unique nucleotide sequence making it possible to distinguish each DPM tag; the green sequence is a sticky end that is capable of ligating to the first Odd nucleotide tag; and the grey sequence is complementary to the First Primer used for library amplification with a part of the grey sequence functioning as a 3′ spacer (3′ Sper).

FIG. 11C is a non-limiting exemplary schematic of an Odd tag (shown in grey) and an Even tag (shown in yellow) ligated together, according to embodiments disclosed herein. Both the Odd and Even tags can be dsDNA molecules which have, as depicted: 1) a 5′ overhang on the top strand that is capable of ligating to either the DPM adaptor (the green sequence in FIG. 2B) or to the 5′ overhang on the bottom strand of the Even tag, 2) both the Odd tag and Even tag have modified 5′ phosphate groups (5′ Phos) to allow for tag elongation, and 3) the bolded regions of complementarity on each tag can be the sequences unique to each of the Odd tags (e.g., 96 Odd tags) and Even tags (e.g., 96 Even tags), resulting in many possible unique sequences amongst both the Odd and Even tags (e.g., 192 unique nucleotide tags).

FIG. 11D is a non-limiting exemplary schematic of a Terminal tag according to embodiments disclosed herein. The Terminal tag as depicted is capable of ligating to an Odd tag and there is no modified 5′ phosphate, making it so that the Terminal tag cannot ligate to itself. As depicted, the Terminal tag has a sequence complementary to a Second Primer (shown in grey) used for library amplification in which the Second Primer anneals to a daughter strand synthesized from a First Primer, and the bolded regions of complementarity on the Terminal tag can be the sequences unique to each of the different Terminal tags, according to embodiments disclosed herein.

FIG. 12A is a non-limiting exemplary schematic showing the molecular biology steps performed for ligating RNA molecules in a cell lysate with a series of unique nucleotide tags. As depicted, RNA is end repaired to obtain a 3′ OH. A partially single-stranded RNA adaptor called RNA Phosphate Modified (RPM) adaptor is ligated to the RNA through a single-stranded RNA ligation. The 3′ end of the RPM adaptor is synthesized with DNA bases and is annealed to a DNA adaptor to generate a double-stranded DNA overhang on the 3′ end of the RPM adaptor. This double-stranded DNA sticky end on RNA allows for ligation of the same set of “Odd” and “Even” tags (as depicted and described in FIG. 2C) to be used for ligation of adaptors to RNA and DNA. A Terminal tag as depicted and described in FIG. 2D is ligated at the last step, and the primer sites can be indicated.

FIG. 12B is a non-limiting exemplary schematic of the RNA Phosphate Modified (RPM) adaptor tags, according to embodiments disclosed herein. The RPM adaptor is designed to specifically ligate RNA molecules using a single-stranded RNA ligase. The features and regions on the RPM as shown, have the following functions: the grey region in the RPM is synthesized using ribonucleotide bases, and it is also a single-stranded overhang on the 5′ end of the molecule that allows for the 5′ end of the RPM molecule to ligate RNA molecules; the pink region serves as a RNA-specific nucleotide tag to identify each read as RNA (if the pink sequence is read) or DNA (if the DPM sequence is read); the blue region may serve as an optionally unique nucleotide sequence making it possible to distinguish each RPM tag from another; the green region of the RPM (which is the same as the green region for the DPM as shown in FIG. 2B), is a sticky end sequence that renders the RPM capable of ligating to a first (e.g., Odd) nucleotide tag; the bottom strand of the RPM is phosphorylated (5 after ligation of the RPM adaptor to DNA to ensure that the RPM adaptor does not form chimeras and ligate to each other; and a 3′ spacer (3′ sper) on the top strand of the RPM adaptor prevents ligation of single-stranded RPM molecules from ligating to the RPM adaptor and forming chimeras of several RPM molecules ligating to each other.

FIG. 12C is a non-limiting exemplary schematic of the amplification of a tagged RNA molecule according to the embodiments disclosed herein. For example, after performing a SPRITE ligation of an RPM adaptor molecule, an Odd nucleotide tag, an Even nucleotide tag, and a Terminal tag on the 3′ end of an RNA molecule in the cell lysate, as depicted in FIG. 2C, FIG. 2D, FIG. 3A, and FIG. 3B, the RNA molecule is converted into cDNA such that a 2P universal primer may be used to amplify the tagged RNA after reverse transcription (RT) in preparation for sequencing of the nucleotide tags.

FIG. 12D is a non-limiting exemplary schematic of the addition (i.e., ligation) of a single stranded (ss)RNA adaptor sequence (shown in blue) ligated to the 5′ end of RNA through a single-stranded RNA ligase, according to embodiments disclosed herein. Using this strategy, after RPM is ligated to an RNA molecule, the bottom strand of the RPM serves as the reverse-transcription primer, and during reverse transcription (+RT), the tagged RNA molecule and the 5′ ssRNA adaptor is converted into cDNA, and the blue region may then serve as a priming site of the 3′ end of the tagged cDNA.

FIG. 12E is a non-limiting exemplary schematic of the ligation of a 2P universal sequence to the cDNA as described and shown in FIG. 3C in which the blue represents a single-stranded DNA adaptor that is ligated to the cDNA through a single-stranded RNA/DNA ligase. Using this strategy, after RPM is ligated, the bottom strand of RPM serves as the reverse-transcription primer, and during reverse transcription (+RT), the tagged RNA is converted into cDNA in which the RNA is then degraded, leaving the cDNA as single-stranded DNA, to which the cDNA adaptor may be ligated through a single-stranded DNA ligation, and the blue region may then serve as a priming site of the 3′ end of the tagged cDNA.

FIG. 12F is a non-limiting exemplary schematic of the addition of a single-stranded adaptor to the cDNA through template switching using a reverse transcriptase that adds the cDNA adaptor to the 3′ end of the cDNA using the Smart-seq strategy, according to embodiments disclosed herein.

FIG. 12G is a non-limiting exemplary schematic of template switching, according to embodiments disclosed herein, in which 1) the reverse transcriptase synthesizes cDNA (shown in orange) and extends leaving 3 dCTP nucleotides (ccc) on the 3′ end of the cDNA, 2) a complementary oligonucleotide with a GGG overhang is hybridized to the CCC sequence on the cDNA, this oligonucleotide also contains a 2P universal priming sequence amplification, and 3) the cDNA is then extended (shown in blue) by the Reverse Transcriptase enzyme to extend the 3′ end of the cDNA to contain the 2P universal priming sequence.

FIG. 13 is a non-limiting exemplary schematic showing the molecular biology steps performed for ligating nucleotide tags to proteins or antibodies, according to embodiments disclosed herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include compositions. In some embodiments, the composition comprises: a plurality of barcoded detection particles. In some embodiments, each barcoded detection particle comprises a particle associated with an antigen-binding protein and a plurality of barcoding oligonucleotides. In some embodiments, the antigen-binding protein is associated with the particle via an immunoglobulin-binding moiety. In some embodiments, plurality of barcoding oligonucleotides comprise a first ligand. In some embodiments, the particle comprises a second ligand. In some embodiments, the plurality of barcoding oligonucleotides are associated with the particle via a multivalent binding agent comprising two or more binding moieties capable of binding the first ligand and/or the second ligand. Disclosed herein include compositions comprising a plurality of barcoded detection particles generated according to the methods disclosed herein.

Disclosed herein include methods for generating barcoded detection particles. In some embodiments, the method comprises: providing a plurality of barcoding oligonucleotides comprising a first ligand; providing a plurality of particles comprising an antigen-binding protein, a second ligand and an immunoglobulin-binding moiety; providing a plurality of multivalent binding agents comprising two or more binding moieties capable of binding the first ligand and/or the second ligand; and/or contacting the plurality of barcoding oligonucleotides, the plurality of particles, and the plurality of multivalent binding agents to generate a plurality of barcoded detection particles, wherein each of the barcoded detection particles comprises a particle associated with a plurality of barcoding oligonucleotides.

Disclosed herein include kits for the generation of barcoded detection particles. In some embodiments, the kit comprises: a plurality of barcoding oligonucleotides comprising a first ligand, or precursor(s) thereof a plurality of particles comprising an antigen-binding protein, a second ligand and an immunoglobulin-binding moiety, or precursor(s) thereof; and/or a plurality of multivalent binding agents comprising two or more binding moieties capable of binding the first ligand and/or the second ligand, or precursor(s) thereof.

Disclosed herein include compositions comprising a pool of barcoded detection particles disclosed herein. In some embodiments, the pool of barcoded detection particles comprises: two or more barcoded detection particles that differ from each other with respect to the antigen-binding protein associated with the particle. In some embodiments, each barcoding oligonucleotide comprises a capture barcode, wherein one or more of the plurality of barcoded detection particles comprises two or more barcoding oligonucleotides having distinct capture barcodes, and wherein each barcoded detection particle of the plurality of barcoded detection particles has a unique set of one or more capture barcode(s) associated therewith specific to the antigen-binding protein associated with the particle to which the barcoding oligonucleotide is associated with. The pool of barcoded detection particles can comprise: at least about 5, about 10, about 20, about 30, about 50, about 75, about 100, about 150, about 200, about 250, about 300, about 350, about 400, about 450, about 500, about 550, about 600, about 650, about 700, about 750, about 800, about 850, about 900, about 950, about 1000, about 2000, about 3000, about 4000, about 5000, about 6000, about 7000, about 8000, about 9000, or about 10000, barcoded detection particles that differ from each other with respect to the antigen-binding protein and barcoding oligonucleotide associated with the particle.

Disclosed herein include methods for detecting interactions between nucleic acid molecules and proteins of interest. In some embodiments, the method comprises: lysing a sample comprising a plurality of cells to generate a cell lysate, wherein the cells comprise nucleic acid molecules suspected of being associated with proteins of interest. The method can comprise: contacting the cell lysate, or a product thereof, with the pool of barcoded detection particles disclosed herein to form a plurality of detection complexes, wherein each of the plurality of detection complexes comprises: a barcoded detection particle; a captured protein of interest; and captured nucleic acid molecule(s) associated with the captured protein of interest. The method can comprise: performing two or more iterations of split-and-pool barcoding, wherein each iteration comprises: (i) randomly distributing the plurality of detection complexes into a plurality of partitions; (ii) in the plurality of partitions, combinatorially barcoding captured nucleic acid molecules and barcoding oligonucleotides, or products thereof, with a combinatorial barcode unit, wherein within each partition, the captured nucleic acid molecules and barcoding oligonucleotides are barcoded with the same combinatorial barcode unit, wherein captured nucleic acid molecules and barcoding oligonucleotides of different partitions receive different combinatorial barcode units from each other, and wherein captured nucleic acid molecules and barcoding oligonucleotides of the same detection complex will assort together in a partition of the plurality of partitions; and (iii) pooling the detection complexes from the plurality of partitions, wherein, after said two or more iterations of split-and-pool barcoding, each combinatorially barcoded captured nucleic acid molecule and each combinatorially barcoded barcoding oligonucleotide comprises a combinatorial barcode comprising two or more combinatorial barcode units, wherein each combinatorial barcode unit corresponds to an iteration of split-and-pool-barcoding. The method can comprise: obtaining sequence information of the combinatorially barcoded captured nucleic acid molecules and the combinatorially barcoded barcoding oligonucleotides, or products thereof. The method can comprise: detecting interactions between captured nucleic acid molecules and proteins of interest based on the sequence information.

Disclosed herein include methods for detecting interactions between ribonucleic acid molecules and RNA-binding proteins (RBPs). In some embodiments, the method comprises: lysing a sample comprising a plurality of cells to generate a cell lysate, wherein the cells comprise ribonucleic acid molecules suspected of being associated with RBPs. The method can comprise: contacting the cell lysate, or a product thereof, with the pool of barcoded detection particles disclosed herein to form a plurality of detection complexes, wherein each of the plurality of detection complexes comprises: a barcoded detection particle; a captured RBP; and captured ribonucleic acid molecule(s) associated with the captured RBP. The method can comprise: converting the captured ribonucleic acid molecule(s) to complementary DNA (cDNA) molecules. The method can comprise: performing two or more iterations of split-and-pool barcoding, wherein each iteration comprises: (i) randomly distributing the plurality of detection complexes into a plurality of partitions; (ii) in the plurality of partitions, combinatorially barcoding cDNA molecules and barcoding oligonucleotides, or products thereof, with a combinatorial barcode unit, wherein within each partition, the cDNA molecules and barcoding oligonucleotides are barcoded with the same combinatorial barcode unit, wherein cDNA molecules and barcoding oligonucleotides of different partitions receive different combinatorial barcode units from each other, and wherein cDNA molecules and barcoding oligonucleotides of the same detection complex will assort together in a partition of the plurality of partitions; and (iii) pooling the detection complexes from the plurality of partitions, wherein, after said two or more iterations of split-and-pool barcoding, each combinatorially barcoded cDNA molecule and each combinatorially barcoded barcoding oligonucleotide comprises a combinatorial barcode comprising two or more combinatorial barcode units, wherein each combinatorial barcode unit corresponds to an iteration of split-and-pool-barcoding. The method can comprise: obtaining sequence information of the combinatorially barcoded cDNA molecules and the combinatorially barcoded barcoding oligonucleotides, or products thereof. The method can comprise: detecting interactions between captured ribonucleic acid molecules and RBPs based on the sequence information.

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, N Y 1989). For purposes of the present disclosure, the following terms are defined below.

### Interaction Detection Compositions, Methods, and Kits

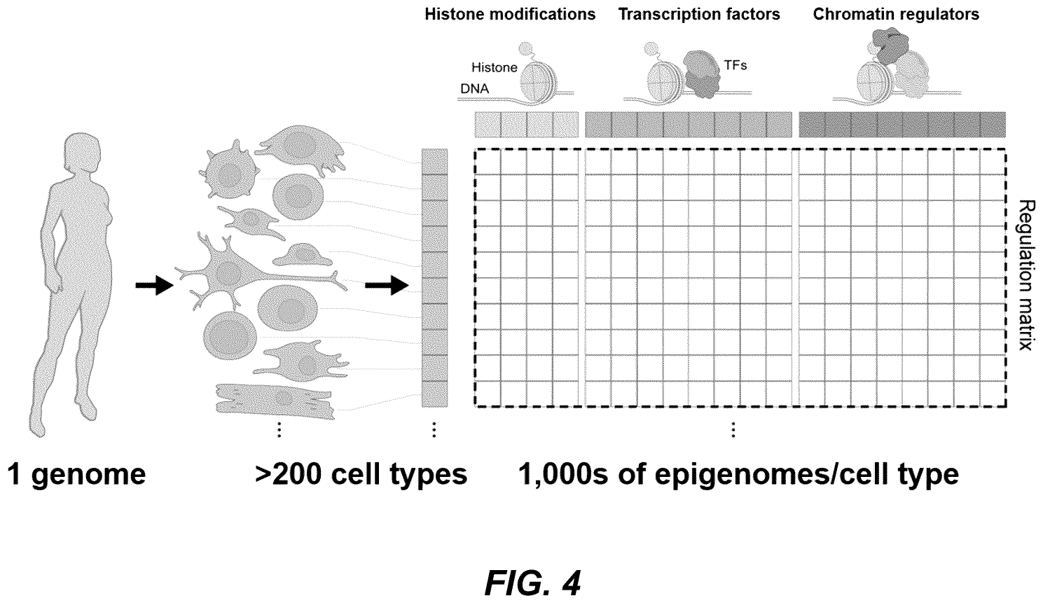

The human body has over 200 cell types, but only one genome. A single genome can encode the information for cell types that have very different functions, in part, based on which regulatory protein is binding where on the genome, and when. The proteins that regulate gene expression can be broken down into a few broad categories: histones, TFs, and CRs. FIG. 4 depicts a non-limiting exemplary schematic related to the challenge of elucidating gene regulation via mapping of proteins on chromatin. The shear number of proteins makes the task of elucidating gene regulation very difficult. Not only is the number of proteins to be examined staggering, but the same protein may work differently depending on the cellular context. Because of the importance of this problem, there have been several international consortiums, such as the ENCODE consortium, that have aimed to address this question by mapping dozens to hundreds of regulatory proteins in different cellular contexts. Despite this effort, this has been focused on cell lines that can be grown in culture, which neglects many other important sample types, such as primary cells and tissues, and rare disease cells. The methods, compositions, and kits provided herein address the need for multiplexed methods of detecting macromolecule interactions, such as, for example, interactions between nucleic acid molecules and proteins of interest. The methods, compositions, and kits provided herein demonstrate advantageous cell type accessibility, can handle protein diversity, and can operate at scale.

Provided herein are compositions, methods, and kits that can enable genome-wide and transcriptome-wide mapping of a large number of proteins or ncRNAs simultaneously, while ensuring that it can be accessed by any lab with low cell number requirements and cost, without the need for specialized equipment.

Some embodiments of the methods provided herein comprise one or both of the following components to uniquely determine protein nucleic acid interactions at scale: (i) Antibody-Bead Generation (hundreds of barcoded beads can be generated, each of which contain antibodies against a specific protein (e.g. chromatin modification, transcription factor, or RBP)); and (ii) Linking Antibodies to DNA/RNA targets. In some embodiments of the methods and compositions provided herein, SPRITE, RD-SPRITE, or other versions thereof provided herein, are employed to add the same barcode to the bead and the captured RNA and/or DNA sequences with which it is associated, and this shared barcode enables assignment of each nucleic acid sequence to a specific bead and therefore enable identification of its associated protein or ncRNA target.

Antibody-Bead Generation: Provided herein is a highly modular and simple scheme to generate hundreds of barcoded antibodies while avoiding key challenges of current methods of oligo-conjugated antibody generation which limit their utility at scale (e.g., multi-step conjugation chemistry, sensitivity to buffer composition and potential oligo interference with antibody activity). The approach can comprise, for example, (1) coupling biotin labeled nucleic acid sequences containing a SPRITE-compatible overhang and unique barcode sequence, and (2) mixing all the different barcoded beads together to generate an antibody pool, which can be stored and used for many parallel experiments. This strategy has several key advantages relative to direct antibody conjugation strategies, such as, but not limited to: (i) because this scheme does not comprise labeling each individual antibody, the approach can work with any antibody of interest and avoids interference of the oligo with epitope binding affinity; (ii) because this scheme integrates multiple labeled barcodes on each bead, the frequency of assigning the protein identity to each target is increased; and (iii) because the approach has fewer barcoded antibodies relative to protein-specific antibodies, fewer sequencing reads of the protein barcodes are needed relative to DNA/RNA to reconstruct their interactions. Moreover, this disclosure provides many ways that this coupling can be done to achieve the goals provided herein.

Linking beads to DNA/RNA targets: In some embodiments of the methods and compositions provided herein, SPRITE, RD-SPRITE, or other versions thereof provided herein, are employed to match the barcoded antibody-bead complex to its associated DNA and RNA targets. Unlike the original SPRITE method, this approach maps barcoded beads relative to genomic DNA or RNA targets. The pooled mapping approach provided herein can comprise one or more of the following steps: (1) crosslinking RNA, DNA, and/or protein interactions within cells (with exact crosslinking conditions determined based on the precise application), and lysing and digesting as appropriate for each assay; (2) incubating the barcoded antibody-bead pools generated above and performing immunoprecipitation; (3) after the appropriate wash steps, performing split-and-pool barcoding to add the same combinatorial barcode to antibody-bead and the captured DNA/RNA sequences; (4) sequencing the combinatorial barcodes linked to each antibody-bead and DNA/RNA sequence; and (5) matching all antibodies and DNA/RNA sequences containing the same barcodes and split the data based on antibody identity to generate a linear localization map for each specific protein.

In summary, each barcoded bead can be seen as an independent IP-seq experiment and the combinatorial barcoding strategy can allow multiplexing of hundreds of different protein targets. The methods, compositions, and kits provided herein can have a number of different applications and advantages over existing methods, including the ability to quickly screen antibodies to find the most high-quality antibody, the ability to generate high-quality maps for any cell type of interest, accessibility to virtually any molecular biology lab, the ability to explore broad classes of regulatory proteins simultaneously, and a fast protocol (less 1 week from start to end). ChIP-DIP/SPIDR can enable a “consortium in a week” via its multiplexed capability.

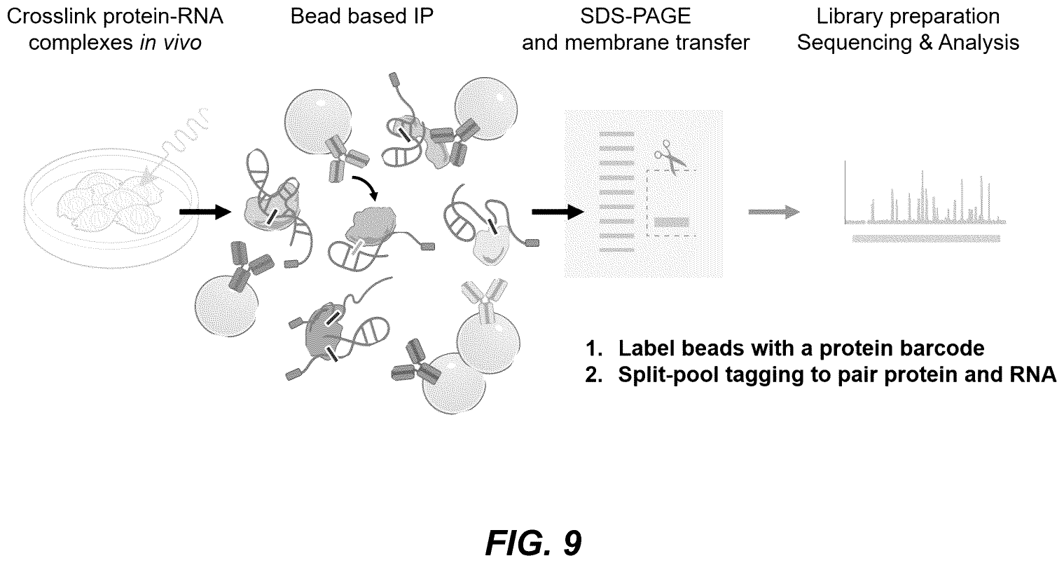

RNA-binding proteins can play crucial roles in regulating all aspects of RNA life/metabolism, from transcription to decay. In addition to RNA biogenesis, RNA binding proteins are also critical for modulating key functions of noncoding RNAs, such as miRNAs and lncRNAs. One prime example of this is Xist, a lncRNA that binds many different proteins on discrete sites of its RNA to enact different functions to orchestrate the process of XCI. Beyond Xist, there are many critical RNAs that are not yet functionally characterized because there is a lack of information regard what proteins they bind and to what sites they bind. There may be thousands of non-canonical RBPs with unknown RNA-binding domains. Recent efforts by the international ENCODE consortium have profiled hundreds of RBPs, yet this only constitutes a small fraction of all putative RBPs identified, and this required dozens of labs and a significant expenditure of time and money. Outstanding questions are how many suspected RBPs are bona fide RBPs, as well as what RNAs they associate with, what precise binding sites, and what are their functional roles. While the method CLIP is seen as a gold standard for confirming and characterizing direct RNA-protein interactions, this approach is limited to studying a single RBP at a time and requires a large number of cells. There are also provided, in some embodiments, methods, compositions, and kits, for detecting interactions between ribonucleic acid molecules and RNA-binding proteins (RBPs) in a highly-multiplexed manner. FIG. 9 depicts a non-limiting exemplary workflow for Split Pool Identification of RBPs (SPIDR).

There are provided, in some embodiments, compositions comprising a plurality of barcoded detection particles. Each barcoded detection particle can comprise a particle associated with an antigen-binding protein and a plurality of barcoding oligonucleotides. The antigen-binding protein can be associated with the particle via an immunoglobulin-binding moiety. The plurality of barcoding oligonucleotides can comprise a first ligand. The particle can comprise a second ligand. The plurality of barcoding oligonucleotides can be associated with the particle via a multivalent binding agent comprising two or more binding moieties capable of binding the first ligand and/or the second ligand.

There are provided, in some embodiments, methods for generating barcoded detection particles. In some embodiments, the method comprises: providing a plurality of barcoding oligonucleotides comprising a first ligand; providing a plurality of particles comprising an antigen-binding protein, a second ligand and an immunoglobulin-binding moiety; providing a plurality of multivalent binding agents comprising two or more binding moieties capable of binding the first ligand and/or the second ligand; and/or contacting the plurality of barcoding oligonucleotides, the plurality of particles, and the plurality of multivalent binding agents to generate a plurality of barcoded detection particles, wherein each of the barcoded detection particles comprises a particle associated with a plurality of barcoding oligonucleotides.

There are provided, in some embodiments, kits for the generation of barcoded detection particles. In some embodiments, the kit comprises: a plurality of barcoding oligonucleotides comprising a first ligand, or precursor(s) thereof; a plurality of particles comprising an antigen-binding protein, a second ligand and an immunoglobulin-binding moiety, or precursor(s) thereof; and/or a plurality of multivalent binding agents comprising two or more binding moieties capable of binding the first ligand and/or the second ligand, or precursor(s) thereof. A precursor of the plurality of barcoding oligonucleotides comprising a first ligand can be, for example, a composition comprising the plurality of barcoding oligonucleotides and a separate composition comprising the first ligand. A precursor of the plurality of particles comprising an antigen-binding protein, a second ligand and an immunoglobulin-binding moiety can be, for example, four separate compositions comprising the antigen-binding protein, the second ligand and the immunoglobulin-binding moiety, respectively.

The first ligand and/or the second ligand can be the same. The first ligand and/or the second ligand can be different. The two or more binding moieties can be the same. At least one of the two or more binding moieties can be different. In some embodiments, the first ligand comprises biotin; the second ligand comprises biotin; the plurality of multivalent binding agents comprise streptavidin; the particle is a Dynabead; and/or the immunoglobulin-binding moiety comprises Protein G. In some embodiments, providing a plurality of barcoding oligonucleotides comprising a first ligand comprises: attaching the first ligand to each of the barcoding oligonucleotides (e.g., via click chemistry). In some embodiments, providing a plurality of particles comprising an antigen-binding protein, a second ligand and an immunoglobulin-binding moiety comprises: attaching the immunoglobulin-binding moiety to the particles (e.g., via click chemistry); attaching the second ligand to the particles (e.g., via click chemistry); and attaching the antigen-binding protein to the particles via the interaction between the antigen-binding protein and the immunoglobulin-binding moiety. In some embodiments, the contacting step comprises: contacting the plurality of multivalent binding agents and the plurality of particles to form first intermediate complexes, and contacting said first intermediate complexes with the plurality of barcoding oligonucleotides to generate the plurality of barcoded detection particles; or contacting the plurality of barcoding oligonucleotides and the plurality of multivalent binding agents to form second intermediate complexes, and contacting said second intermediate complexes with the plurality of plurality of particles to generate the plurality of barcoded detection particles.

The first ligand and at least one of the two or more binding moieties can be a specific binding pair, wherein the specific binding pair comprises a first member of a specific binding pair and a second member of a specific binding pair that bind one another with: (i) high affinity, high avidity, and/or high specificity, or (ii) low affinity, low avidity, and/or low specificity. The second ligand and at least one of the two or more binding moieties can be a specific binding pair, wherein the specific binding pair comprises a first member of a specific binding pair and a second member of a specific binding pair that bind one another with: (i) high affinity, high avidity, and/or high specificity, or (ii) low affinity, low avidity, and/or low specificity. In some embodiments, the binding between the first and second member of the specific binding pair occurs via covalent bonding. In some embodiments, the binding between the first and second member of the specific binding pair occurs via non-covalent interactions. The non-covalent interactions can comprise one or more of ionic bonding, hydrophobic interactions, van der Waals forces, and hydrogen bonding. The binding between the first and the second member of the specific binding pair can have a dissociation constant K_{d} between about 10⁻¹⁰ to about 10⁻¹⁵ mol/L.

The first and the second member of the specific binding pair can be each selected from the group comprising an antibody or an antigen-binding portion thereof and an antigen, an biotin moiety and an avidin moiety, a dinitrophenol (DNP) and an anti-DNP antibody, a digoxin and an anti-digoxin antibody, a digoxigenin and an anti-digoxigenin antibody, a hapten and an anti-hapten, a polysaccharide and a polysaccharide binding moiety, a lectin and a receptor, a ligand and a receptor, a fluorescein and an anti-fluorescein antibody, complementary nucleic acids, derivatives therefore, and fragments thereof.

The multivalent binding agent, the first ligand, the second ligand, and/or at least one of the two or more binding moieties can be a biotin moiety and/or an avidin moiety. The biotin moiety can be selected from the group comprising biotin (cis-hexahydro-2-oxo-1H-thieno[3,4]imidazole-4-pentanoic acid) and derivatives or analogs thereof that can specifically bind to an avidin moiety. The biotin moiety can be selected from the group comprising biotin-e-N-lysine, biocytin hydrazide, amino or sulfhydryl derivatives of 2-iminobiotin and biotinyl-6-aminocaproic acid-N-hydroxysuccinimide ester, sulfosuccinimideiminobiotin, biotinbromoacetylhy dr azide, p-diazobenzoyl biocytin, 3-(N-maleimidopropionyl)biocytin, oxybiotin, 2′-iminobiotin, diaminobiotin, biotin sulfoxide, biocytin, 9-methylbiotin, biotin methyl ester (MEBio), desthiobiotin (DEBio), e-N-Biotinyl-L-lysine, diaminobiotin (DABio), biotin sulfone, 2′-thiobiotin and N3′-ethyl biotin, and derivatives thereof. The avidin moiety can comprise native egg-white glycoprotein avidin, or any derivatives, analogs, fragments and other non-native forms thereof that can specifically bind to a biotin moiety. The avidin moiety can comprise an N-acyl avidin. The N-acyl avidin can comprise N-acetyl avidin, N-phthalyl avidin, N-succinyl avidin, and derivatives thereof. The avidin moiety can comprise streptavidin, nitrostreptavidin, ExtrAvidin, Captavidin, Neutravidin, Neutralite Avidin, and derivatives thereof.

The immunoglobulin-binding moiety can comprise Protein L, Protein G, Protein A, Protein A/G, or a combination thereof. The antigen binding protein can comprise an antibody, an antibody fragment, an scFv, a Fv, a Fab, a (Fab′)2, a single domain antibody (SDAB), a VH or VL domain, a camelid VHH domain, a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. The antigen binding protein can be capable of specifically binding a protein of interest. The protein of interest can be an RNA-binding protein. The RBP can be a suspected RBP or a confirmed RBP. The antigen-binding protein can be capable of binding to a histone protein, including a modified histone protein, such as, for example, a histone tail that has been modified by one or more of acetylation, methylation, phosphorylation, and ubiquitination. In some embodiments, the antigen binding protein is not conjugated to an oligonucleotide. In some embodiments, the protein of interest is: a histone modification (e.g., H2AZK4/K7Ac, H2BK12Ac, H2BK15Ac, H2BK20Ac, H3K14Ac, H3K18Ac, H3K9Ac, H3K27Ac, H3K36Ac, H3K56Ac, H3K9/K14Ac, H4K5Ac, H4K12Ac, H4K16Ac, H3 Ser10p, H3Thr3p, H2AK119ub, H2AK120ub, H3K4me1, H3K79me1, H3K9me1, H3K27me2, H3K4me2, H3K79me2, H3K9me2, H3K9me2/me3, H3K4me3, H3K36me3, H3K36me1, H3K36me2, H3K79me3, H3K9me3, H4K20me3, H3R8me2, H3R3me2, H3R18me2, or any combination thereof); and/or chromatin-associated proteins, such as transcription factors, chromatin regulators or polymerases, and their modified forms (e.g., AEBP2, ATF2, BCL6, Beta Catenin, CBFβ, CDK8 NELFb, CREB, CTCF, DNMT3A, DNMT3B, E2F1, E2F4, EGR1, ELK1, ELL, FoxP1, HIF 1, INTS9, KLF5, LAP1α, LAP1β, MAX, MAZ, MBD2, MBD3, MITF, MNT, MeCP2, NRF1, Nanog, Pou5f1, RAD21, RBPJ, RFX1, RNF20, RING1, SP1, SPT16, SRF, Suz12, Sox2, TAF1, TBP, TCF4, TET1, TET2, TH1L, USF2, UTX, YY1, ZNF24, ZNF687, cFos, cFos-pSer32, cJun, cJun-pSer63, cJun-pSer73, P53, P53-pSer15, POLR1A, POLR2A, POLR2A-pSer2, POLR2A-pSer5, POLR2A-pSer2/5, POLR3A, POLR2A-pThr4, POLR3D, POLR3E, ASH2, BAF57, BRD3, BRD4, BRG1, CBP, CLOCK, ESET, EZH2, G9a, HDAC1, HDAC2, HDAC3, HDAC5, HDAC6, HP1α, HP1β, JARID1A, JARID1B, JARID2, JMJD2A, LSD1, MLL, MTA1, MTA2, Menin, NFRkB, PCAF, PHC1, PHF8, RBBP5, RING1B, SAP30, SETD1A, SETD2, SIN3A, SIRT6, SPT4, SPT6, SRC3, SSRP1, WDR5, ZMYND11, or any combination thereof). In some embodiments, the protein of interest is: a transcriptional regulator (e.g., ILF3, SAF-B, TAF15, EWSR1, WDR43, TARDBP, FUBP3, SSB, SLBP, ELAVL1, SHARP, FUS, RBM15, SAF-A, TIAL1, LBR, or any combination thereof); an RNA processing factor (e.g., CPSF6, hnRNPC, hnRNPL, KHSRP, LIN28B, PTBP1, QKI, RBM5, U2AF1, hnRNPM, NOLC1, TRA2A, BUD13, RBFOX2, DROSHA, DGCR8, LSM11, SMNDC1, hnRNPA1, DDX52, DDX55, AQR, SRSF9, or any combination thereof); and/or a translational regulator (e.g., PCBP1, PCBP2, IGF2BP1, IGF2BP2, IGF2BP3, RPS3, UPF1, LARP1, FASTKD2, LARP4, LARP7, DDX6, PUM1, DHX30, or any combination thereof).

The particle can be or can comprise a bead, a gold bead, polysaccharide, poly(acrylate), polystyrene, poly(acrylamide), polyol, agarose, agar, cellulose, dextran, starch, heparin, glycogen, amylopectin, mannan, inulin, nitrocellulose, diazocellulose, polyvinylchloride, polypropylene, polyethylene, nylon, a latex bead, a conducting metal, a nonconducting metal, glass, a magnetic bead, a paramagnetic bead, a superparamagnetic bead, or any combination thereof. Each particle can be associated with at least about 2, about 3, about 4, about 5, about 6, about 7, about 8, about 9, about 10, about 11, about 12, about 14, about 16, about 18, about 20, about 30, about 40, about 50, about 60, about 70, about 80, or about 100, barcoding oligonucleotides. Each barcoding oligonucleotide can comprise a capture barcode, and the capture barcode can be a unique sequence specific to the antigen-binding protein associated with the particle to which the barcoding oligonucleotide is associated with.

The first ligand can be situated: (i) on the 3′ end of the barcoding oligonucleotide, (ii) on the 5′ end of the barcoding oligonucleotide, and/or (iii) internally between the 5′ end and the 3′ end of the barcoding oligonucleotide. The 5′ end of the barcoding oligonucleotide can comprise a modified phosphate group and/or a 5′ overhang capable of ligation to the 5′ overhang of a combinatorial barcode unit. The barcoding oligonucleotide can comprise a unique molecular identifier (UMI), which can be about 8 nucleotides in length. The barcoding oligonucleotide can comprise a universal library sequence. The universal library sequence can comprise a sequence complementary to at least a portion of a sequencing primer, such as, for example, an Illumina-compatible sequencing primer sequence (e.g., a Read 1 sequencing primer or a Read 2 sequencing primer). The barcoding oligonucleotide can further comprise a spacer sequence (e.g., a 3′ spacer sequence).

There are provided, in some embodiments, compositions comprising a plurality of barcoded detection particles generated according to the methods disclosed herein. There are provided, in some embodiments, compositions comprising a pool of barcoded detection particles disclosed herein. In some embodiments, the pool of barcoded detection particles comprises: two or more barcoded detection particles that differ from each other with respect to the antigen-binding protein associated with the particle. In some embodiments, each barcoding oligonucleotide comprises a capture barcode, wherein one or more of the plurality of barcoded detection particles comprises two or more barcoding oligonucleotides having distinct capture barcodes, and wherein each barcoded detection particle of the plurality of barcoded detection particles has a unique set of one or more capture barcode(s) associated therewith specific to the antigen-binding protein associated with the particle to which the barcoding oligonucleotide is associated with. The pool of barcoded detection particles can comprise: at least about 5, about 10, about 20, about 30, about 50, about 75, about 100, about 150, about 200, about 250, about 300, about 350, about 400, about 450, about 500, about 550, about 600, about 650, about 700, about 750, about 800, about 850, about 900, about 950, about 1000, about 2000, about 3000, about 4000, about 5000, about 6000, about 7000, about 8000, about 9000, or about 10000, barcoded detection particles that differ from each other with respect to the antigen-binding protein and barcoding oligonucleotide associated with the particle.

There are provided, in some embodiments, methods for detecting interactions between nucleic acid molecules and proteins of interest. In some embodiments, the method comprises: lysing a sample comprising a plurality of cells to generate a cell lysate, wherein the cells comprise nucleic acid molecules suspected of being associated with proteins of interest. The method can comprise: contacting the cell lysate, or a product thereof, with the pool of barcoded detection particles disclosed herein to form a plurality of detection complexes, wherein each of the plurality of detection complexes comprises: a barcoded detection particle; a captured protein of interest; and captured nucleic acid molecule(s) associated with the captured protein of interest.

The method can comprise: performing two or more iterations of split-and-pool barcoding, wherein each iteration comprises: (i) randomly distributing the plurality of detection complexes into a plurality of partitions; (ii) in the plurality of partitions, combinatorially barcoding captured nucleic acid molecules and barcoding oligonucleotides, or products thereof, with a combinatorial barcode unit, wherein within each partition, the captured nucleic acid molecules and barcoding oligonucleotides are barcoded with the same combinatorial barcode unit, wherein captured nucleic acid molecules and barcoding oligonucleotides of different partitions receive different combinatorial barcode units from each other, and wherein captured nucleic acid molecules and barcoding oligonucleotides of the same detection complex will assort together in a partition of the plurality of partitions; and (iii) pooling the detection complexes from the plurality of partitions, wherein, after said two or more iterations of split-and-pool barcoding, each combinatorially barcoded captured nucleic acid molecule and each combinatorially barcoded barcoding oligonucleotide comprises a combinatorial barcode comprising two or more combinatorial barcode units, wherein each combinatorial barcode unit corresponds to an iteration of split-and-pool-barcoding.

The method can comprise: obtaining sequence information of the combinatorially barcoded captured nucleic acid molecules and the combinatorially barcoded barcoding oligonucleotides, or products thereof. The method can comprise: detecting interactions between captured nucleic acid molecules and proteins of interest based on the sequence information.

In some embodiments, the method further comprises: adding a crosslinking agent (e.g., formaldehyde) to the plurality of cells prior to the lysis step; or adding a crosslinking agent to the cell lysate. In some embodiments, the method further comprises: isolation of the nuclei of the plurality of cells. The nuclei of the plurality of cells can be obtained via centrifugation of the cell lysate. In some embodiments, the method further comprises: fragmentation of the chromatin of the plurality of cells. Fragmentation can comprise enzymatic chromatin fragmentation and/or sonication of the nuclear pellet. In some embodiments, said fragmentation generates chromatin fragments of about 150 bp to about 700 bp, and can have an average size of about 350 bp. In some embodiments, the method further comprises: processing at least one end of the captured nucleic acid molecule(s) to enable ligation of said captured nucleic acid molecule(s) to a ligation adaptor molecule. Said processing can comprise blunt-ending, phosphorylation, and/or dA-tailing. In some embodiments, the method further comprises: ligating a ligation adaptor molecule (e.g., an DNA Phosphate Modified (DPM) tag) to the captured nucleic acid molecule(s).

A probability of the interaction between the nucleic acid molecule and the protein of interest as being bona fide can be proportional to the number of iterations of split-and-pool barcoding. Performing two or more iterations of split-and-pool barcoding can comprise performing n*2 iterations of split-and-pool barcoding, n can be an integer greater than zero, The combinatorial barcode unit can be an Odd tag or an Even tag, and each set of two iterations can comprise barcoding with an Odd tag followed by barcoding with an Even tag. Performing two or more iterations of split-and-pool barcoding can comprise performing n iterations of split-and-pool barcoding, and n can be an integer greater than one. In some embodiments, n can be at least about 2, about 3, about 4, about about 6, about 7, about 8, about 9, about 10, about 11, about 12, about 14, about 16, about 18, about about 30, about 40, about 50, about 60, about 70, about 80, or about 100.

Each combinatorial barcode unit can comprise at least one 5′ overhang, and said 5′ overhang can be capable of ligating to a 5′ overhang of one or more of a ligation adaptor molecule, a combinatorial barcode unit, or a terminal tag. Each combinatorial barcode unit can comprise a modified 5′ phosphate group. In some embodiments, the combinatorial barcoding step comprises: annealing the 5′ overhang of a barcoding oligonucleotide, a ligation adaptor molecule, or a combinatorial barcode unit, to the 5′ overhang of a combinatorial barcode unit; and ligating the annealed molecules. In some embodiments, the method further comprises, following the two or more iterations of split-and-pool barcoding: annealing a terminal tag to each captured nucleic acid molecule and each barcoding oligonucleotide; and ligating said annealed molecules. The barcoding oligonucleotide and/or terminal tag can further comprise a spacer sequence, such as, for example, a 3′ spacer sequence that allows the combinatorial barcode unit to only ligate to the 5′ end of each single-stranded DNA sequence and prevents formation of hairpins during library amplification.

In some embodiments, the method further comprises: reversing crosslinking to elute the combinatorially barcoded captured nucleic acid molecules and the combinatorially barcoded barcoding oligonucleotides from the particles. Obtaining sequence information can comprise amplifying the combinatorially barcoded captured nucleic acid molecules and the combinatorially barcoded barcoding oligonucleotides. In some embodiments, obtaining sequence information comprises: obtaining sequencing data comprising a plurality of sequencing reads of the combinatorially barcoded captured nucleic acid molecules and the combinatorially barcoded barcoding oligonucleotides, or products thereof. Each of the plurality of sequencing reads of the combinatorially barcoded captured nucleic acid molecules, or products thereof, can comprise: a combinatorial barcode sequence, and a captured nucleic acid molecule sequence; and Each of the plurality of sequencing reads of the combinatorially barcoded barcoding oligonucleotides, or products thereof, can comprise: a combinatorial barcode sequence, and a capture barcode sequence, wherein the capture barcode is a unique sequence specific to the antigen-binding protein associated with the particle to which the barcoding oligonucleotide is associated with.

In some embodiments, detecting interactions comprises: for each unique combinatorial barcode sequence, which indicates a single detection complex of the plurality of detection complexes, identifying the captured nucleic acid molecule sequence and capture barcode sequence of sequencing reads sharing a combinatorial barcode sequence. In some embodiments, detecting interactions comprises: for each unique capture barcode sequence, which indicates a captured protein of interest, identifying the captured nucleic acid molecule sequence of sequencing reads sharing a capture barcode sequence. In some embodiments, the method comprises determining the binding site of a captured protein of interest on associated captured nucleic acid molecule sequence(s). The method can further comprise aligning captured nucleic acid molecule sequence(s) to a reference genome. The nucleic acid molecules can comprise deoxyribonucleic acid molecules and/or ribonucleic acid molecules. The nucleic acid molecules can be selected from the group comprising double-stranded DNA, single-stranded DNA, microRNA (miRNA), messenger RNA (mRNA), long non-coding RNA (lncRNA), ribosomal RNA (rRNA), transfer RNA (tRNA), small nuclear RNA (snRNA), small nucleolar RNA (snoRNA), Piwi-interacting RNA (piRNA), interfering RNA (siRNA), antisense RNA (aRNA), transfer messenger RNA (tmRNA), tRNA-derived small RNA (tsRNA), rDNA-derived small RNA (srRNA), ribozyme, viral RNA, single-stranded RNA, double-stranded RNA, or any combination thereof. Detecting interactions between nucleic acid molecules and proteins of interest can comprise detecting interactions between nucleic acid molecules and at least about 2, about 4, about 6, about 8, about 10, about 12, about 14, about 16, about 18, about about 30, about 40, about 50, about 60, about 70, about 80, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 2000, about 3000, about 4000, about 5000, about 6000, about 7000, about 8000, about 9000, or about 10000, different proteins of interest.

There are provided, in some embodiments, methods for detecting interactions between ribonucleic acid molecules and RNA-binding proteins (RBPs). In some embodiments, the method comprises: lysing a sample comprising a plurality of cells to generate a cell lysate, wherein the cells can comprise ribonucleic acid molecules suspected of being associated with RBPs. The method can comprise: contacting the cell lysate, or a product thereof, with the pool of barcoded detection particles disclosed herein to form a plurality of detection complexes, wherein each of the plurality of detection complexes comprises: a barcoded detection particle; a captured RBP; and captured ribonucleic acid molecule(s) associated with the captured RBP. The method can comprise: converting the captured ribonucleic acid molecule(s) to complementary DNA (cDNA) molecules.

The method can comprise: performing two or more iterations of split-and-pool barcoding, wherein each iteration comprises: (i) randomly distributing the plurality of detection complexes into a plurality of partitions; (ii) in the plurality of partitions, combinatorially barcoding cDNA molecules and barcoding oligonucleotides, or products thereof, with a combinatorial barcode unit, wherein within each partition, the cDNA molecules and barcoding oligonucleotides are barcoded with the same combinatorial barcode unit, wherein cDNA molecules and barcoding oligonucleotides of different partitions receive different combinatorial barcode units from each other, and wherein cDNA molecules and barcoding oligonucleotides of the same detection complex will assort together in a partition of the plurality of partitions; and (iii) pooling the detection complexes from the plurality of partitions, wherein, after said two or more iterations of split-and-pool barcoding, each combinatorially barcoded cDNA molecule and each combinatorially barcoded barcoding oligonucleotide comprises a combinatorial barcode comprising two or more combinatorial barcode units, wherein each combinatorial barcode unit corresponds to an iteration of split-and-pool-barcoding.

The method can comprise: obtaining sequence information of the combinatorially barcoded cDNA molecules and the combinatorially barcoded barcoding oligonucleotides, or products thereof. The method can comprise: detecting interactions between captured ribonucleic acid molecules and RBPs based on the sequence information. In some embodiments, the method further comprises: crosslinking interacting ribonucleic acids and RBPs (e.g., via UV crosslinking and/or via a crosslinking agent). Said UV crosslinking can comprise about 0.01 J cm⁻² to about 25 J cm⁻² of UV at about 100 nm to about 400 nm, such as, for example, about 0.25 J cm⁻² (UV 2.5k) of UV at about 254 nm. The crosslinking step can comprise contacting (e.g., contacting samples, cells, detection complexes) with 4-Thiouridine (4SU) and/or 6-thioguanosine (6SG).

The method further can comprise DNase digestion and/or sonication of the cell lysate. In some embodiments, the method further comprises: partial fragmentation of the ribonucleic acids of the plurality of cells The partial fragmentation can be enzyme-mediated (e.g., via RNase If). Said partial fragmentation can generate ribonucleic acids of about 300 bp to about 400 bp in length. In some embodiments, the method further comprises: processing the 3′ ends of captured ribonucleic acid molecules to enable ligation of said captured ribonucleic acid molecules to a ligation adaptor molecule. Said processing can comprises end repair (e.g., using T4 Polynucleotide Kinase). End repair can comprise processing said captured ribonucleic acid molecules to have 3′ OH groups compatible for ligation. In some embodiments, the method further comprises: ligating a ligation adaptor molecule (e.g., an RNA Phosphate Modified (RPM) tag) to the captured ribonucleic acid molecules. Ligating can comprise use of RNA Ligase I. Converting the captured ribonucleic acid molecule(s) to complementary DNA (cDNA) molecules can be performed after ligation of the ligation adaptor molecule using a reverse transcription primer having a 5′ overhang capable of ligation to the overhang of a combinatorial barcode unit.

A probability of the interaction between the ribonucleic acid molecule and the RBP as being bona fide can be proportional to the number of iterations of split-and-pool barcoding. Performing two or more iterations of split-and-pool barcoding can comprise performing n*2 iterations of split-and-pool barcoding, and n can be an integer greater than zero. The combinatorial barcode unit can be an Odd tag or an Even tag, and each set of two iterations can comprise barcoding with an Odd tag followed by barcoding with an Even tag. Performing two or more iterations of split-and-pool barcoding can comprise performing n iterations of split-and-pool barcoding, and n can be an integer greater than one. In some embodiments, n can be at least about 2, about 3, about 4, about 5, about 6, about 7, about 8, about 9, about 10, about 11, about 12, about 14, about 16, about 18, about 20, about about 40, about 50, about 60, about 70, about 80, or about 100.

Each combinatorial barcode unit can comprise at least one 5′ overhang, and said overhang can be capable of ligating to a 5′ overhang of one or more of a reverse transcription primer, a combinatorial barcode unit, or a terminal tag. Each combinatorial barcode unit can comprise a modified 5′ phosphate group. In some embodiments, the combinatorial barcoding step comprises: annealing the 5′ overhang of a barcoding oligonucleotide, a reverse transcription primer, or a combinatorial barcode unit, to the 5′ overhang of a combinatorial barcode unit; and ligating the annealed molecules. In some embodiments, the method further comprises, following the two or more iterations of split-and-pool barcoding: annealing a terminal tag to each cDNA molecule and each barcoding oligonucleotide; and ligating said annealed molecules. The barcoding oligonucleotide and/or terminal tag can further comprise a spacer sequence, such as, for example, a 3′ spacer sequence that allows the combinatorial barcode unit to only ligate to the 5′ end of each single-stranded DNA sequence and prevents formation of hairpins during library amplification. In some embodiments, the method further comprises: reversing crosslinking to elute the combinatorially barcoded cDNA molecules and the combinatorially barcoded barcoding oligonucleotides from the particles.

Obtaining sequence information can comprise amplifying the combinatorially barcoded cDNA molecules and the combinatorially barcoded barcoding oligonucleotides. In some embodiments, the amplifying step comprises: ligating a chimeric ssDNA-dsDNA adaptor to the 3′ ends of the combinatorially barcoded cDNA molecules via a splint ligation reaction. Said chimeric ssDNA-dsDNA adaptor can comprise a random sequence that anneals to the 3′ end of the cDNA.

In some embodiments, obtaining sequence information comprises: obtaining sequencing data comprising a plurality of sequencing reads of the combinatorially barcoded cDNA molecules and the combinatorially barcoded barcoding oligonucleotides, or products thereof. Each of the plurality of sequencing reads of the combinatorially barcoded cDNA molecules, or products thereof, can comprise: a combinatorial barcode sequence, and a captured cDNA molecule sequence. Each of the plurality of sequencing reads of the combinatorially barcoded barcoding oligonucleotides, or products thereof, can comprise: a combinatorial barcode sequence, and a capture barcode sequence, wherein the capture barcode is a unique sequence specific to the antigen-binding protein associated with the particle to which the barcoding oligonucleotide is associated with.

In some embodiments, detecting interactions comprises: for each unique combinatorial barcode sequence, which indicates a single detection complex of the plurality of detection complexes, identifying the captured cDNA molecule sequence and capture barcode sequence of sequencing reads sharing a combinatorial barcode sequence. In some embodiments, detecting interactions comprises: for each unique capture barcode sequence, which indicates a captured RBP, identifying the captured cDNA molecule sequence of sequencing reads sharing a capture barcode sequence. The method can further comprise determining the binding site of a captured RBP on associated captured ribonucleic acid molecule sequence(s). The method can further comprise aligning captured cDNA molecule sequence(s) to a reference genome. The ribonucleic acid molecules can be selected from the group comprising microRNA (miRNA), messenger RNA (mRNA), long non-coding RNA (lncRNA), ribosomal RNA (rRNA), transfer RNA (tRNA), small nuclear RNA (snRNA), small nucleolar RNA (snoRNA), Piwi-interacting RNA (piRNA), interfering RNA (siRNA), antisense RNA (aRNA), transfer messenger RNA (tmRNA), tRNA-derived small RNA (tsRNA), rDNA-derived small RNA (srRNA), ribozyme, viral RNA, single-stranded RNA, double-stranded RNA, or any combination thereof. Detecting interactions between ribonucleic acid molecules and RBPs can comprise detecting interactions between ribonucleic acid molecules and at least about 2, about 4, about 6, about 8, about 10, about 12, about 14, about 16, about 18, about 20, about 30, about 40, about 50, about 60, about 70, about 80, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 2000, about 3000, about 4000, about 5000, about 6000, about 7000, about 8000, about 9000, or about 10000, different RBPs.

The contacting step can comprise immunoprecipitation. The plurality of partitions can be in fluid isolation from each other and/or can comprise wells, microwells, tubes, vials, microcapsules, droplets, or any combination thereof. The plurality of partitions can comprise at least about 6, about 8, about 10, about 12, about 14, about 16, about 18, about 20, about 22, about 24, about 26, about 28, about 30, about 32, about 34, about 36, about 38, about 40, about 42, about 44, about 46, about 48, about 50, about 52, about 54, about 56, about 58, about 60, about 62, about 64, about 66, about 68, about 70, about 72, about 74, about 76, about 78, about 80, about 82, about 84, about 86, about 88, about 90, about 92, about 94, about 96, about 98, or about 100, different partitions comprising different combinatorial barcode units from each other.

The sample can be obtained from a subject, such as, for example, an organ of the subject, a tissue of the subject, or a bodily fluid of the subject. The plurality of cells can comprise immortalized cells and/or primary cells. The plurality of cells can comprise a eukaryotic cell. The eukaryotic cell can comprise an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof. The plurality of cells can comprise cells of human origin or cells of non-human origin, such as, for example, mouse cells, rat cells, rabbit cells, pig cells, bovine cells, primate cells, non-mammalian cells, fish cells, insect cells, mold cells, dictostelium cells, worm cells, or drosophila cells.

In some embodiments, lysing a sample comprising a plurality of cells can comprise lysing a plurality of samples each comprising a plurality of cells. In some embodiments, the plurality of samples differ with respect to cell type. Detection complexes of the same sample can be labeled with a nucleic acid comprising the same unique sample identifier sequence, wherein detection complexes of different samples differ with respect to the unique sample identifier sequence added during said labeling. The method can comprise pooling the detection complexes of different samples after said labeling step and prior to performing two or more iterations of split-and-pool barcoding. The ligation adaptor molecule can comprise a unique sample identifier sequence. Each of the sequencing reads of the combinatorially barcoded captured nucleic acid molecule, the combinatorially barcoded cDNA molecules and/or the combinatorially barcoded barcoding oligonucleotides, or products thereof, can comprise a unique sample identifier sequence. The method can comprise identifying the sample origin of detection complexes based on the unique sample identifier sequence of one or more sequencing reads originating from said detection complexes.

### Detecting Interactions Between Macromolecules and Candidate Interaction Partners

Almost all detection methods for proteins utilize affinity reagents such as antibodies and aptamers. Yet, there are still a limited number of high-quality affinity reagents for most proteins. A reason for this is that conventional methods for screening libraries of molecules that bind to proteins, or that interfere with protein interactions, are typically low-throughput, labor intensive, and expensive. Thus, the ability to perform high-throughput screening of affinity reagents for specific proteins has conventionally been limited, and has conventionally resulted in a bottle-neck in the numbers of affinity reagents that are available. As an example, a complex library of in vitro generated affinity reagents (>10¹³ combinations) may be readily generated, yet evaluating which sequence binds a specific protein through individual interaction assays would involve billions of individual assays, which is not amendable to conventional screening platforms. There are provided, in some embodiments, methods, compositions, and kits for detecting interactions between macromolecules and candidate interaction partners.

As used herein, “macromolecule” has its customary and ordinary meaning as would be understood by one of ordinary skill in the art in view of this disclosure. It refers to a relatively large molecule such as a protein or nucleic acid such as RNA or DNA. It will be appreciated that macromolecules may be part of a larger complex, for example, a protein complex, or a protein-RNA complex, or a protein-DNA complex. Example macromolecules suitable for embodiments herein can comprise, consist essentially of, or consist of proteins, peptides, RNA binding proteins, chromatin associated proteins, enzymes, receptors, ligands, aptamers, immune cell receptors such as T cell receptors, antibodies, and antibody fragments such as Fabs, minibodies, diabodies, single chain variable fragments (scFvs), and nanobodies, or a combination of two or more of any of the listed items. In some embodiments, macromolecules are translated in vitro. The systems, methods, compositions, and kits provided herein can, in some embodiments, be employed in concert with the systems, methods, compositions, and kits described in U.S. Patent Application Publication No. 2020/0087657, the content of which is incorporated herein by reference in its entirety.

As used herein, “candidate interaction partner” has its customary and ordinary meaning as would be understood by one of ordinary skill in the art in view of this disclosure. It refers to a molecule that may interact with a macromolecule as described herein. In detecting methods and kits of some embodiments, binding between macromolecules and candidate interaction partners is detected. For example, a library of candidate interaction partners may be screened for binding to (or inhibition of complex formation for) one or more macromolecules. Examples candidate interaction partners suitable for embodiments herein can comprise, consist essentially of, or consist of proteins, peptides, DNA, RNA, and small molecules. In some embodiments, candidate interaction partners are transcribed and/or translated.

In some embodiments, methods of detecting an interaction between a macromolecule and an interaction partner are described. For conciseness, these methods may be referred to herein as “detecting methods.” In some embodiments, the barcoded detection particles provided herein do not comprise an antigen-binding protein and/or an immunoglobulin-binding moiety. In some embodiments, the barcoded detection particles provided herein are instead associated with macromolecule(s) and/or candidate interaction partner(s). In some embodiments, the method comprises obtaining a pool of macromolecules (for example, a library of antigen binding proteins such as nanobodies) and/or a pool of candidate interaction partners (for example, a library of candidate binding targets). In some embodiments, macromolecules and/or candidate interaction partners are associated with a barcoded particle. Each barcoded particle can be associated with a different macromolecule of the pool of macromolecules, or a different candidate interaction partner of the pool of candidate interaction partners. The method can comprise contacting the pool of barcoded detection complexes with macromolecules and/or candidate interaction partners to allow interaction to occur (if possible). The detecting method can comprise performing two or more iterations of split-and-pool barcoding. Each iteration of split-and-pool barcoding can comprise (i) randomly distributing the barcoded detection complexes into a plurality of partitions in fluid isolation from each other. The iteration of split-and-pool barcoding can further comprise (ii) barcoding the macromolecules and candidate interaction partners in the partitions with a combinatorial barcode unit as described herein, so that within each partition, the macromolecules and candidate interaction partners are barcoded with the same combinatorial barcode unit.

In some embodiments, the macromolecules and/or candidate interaction partners comprise a first ligand and/or second ligand, and are capable of being associated with a plurality of barcoding oligonucleotides via a multivalent binding agent comprising two or more binding moieties capable of binding the first ligand and/or the second ligand. Detecting methods and kits of some embodiments herein permit high-throughput identification of protein-protein, protein-DNA, and protein-RNA interactions to screen highly complex libraries of billions of molecules (such as affinity reagents) that bind to proteins or libraries of molecules (including proteins, RNAs, small molecules, etc.) that interfere with protein interactions in a single experiment. The detecting methods can make use of combinatorial barcoding scheme via the addition of tags onto each affinity reagent and macromolecule.

In the detecting method of some embodiments, each macromolecule is a protein, and each macromolecule comprises an identifier barcode comprising an polynucleotide comprising a coding sequence of the macromolecule, for example an mRNA encoding the macromolecule. The identifier barcode may further comprise a covalent polypeptide tag fused to the polynucleotide, and the protein may further comprise a counterpart polypeptide sequence covalently bound to the covalent polypeptide tag. In the detecting method of some embodiments, the counterpart polypeptide sequence is disposed at an N-terminal region of the macromolecule (protein). The detecting method of some embodiments further comprises fusing the covalent polypeptide tag to the polynucleotide encoding the macromolecule, and translating the polynucleotide in vitro, thus producing the macromolecule comprising the counterpart polypeptide sequence disposed at an N-terminal portion of the macromolecule. The detecting method may further comprise covalently binding the polypeptide tag to the counterpart polypeptide sequence, thus making the macromolecule comprising the identifier barcode. Examples of covalent polypeptide tag and counterpart polypeptide sequences suitable for detecting methods herein include, but are not limited to, a split CnaB protein; or a Spytag and SpyCatcher; or Isopeptag and pilin-C; or SnoopTag and SnoopCatcher; or DogTag and SnoopTagJr; or SdyTag and SdyCatcher, or a combination of two or more of any of the listing pairs. For example, the covalent polypeptide tag and counterpart polypeptide sequences may comprise a Spytag and SpyCatcher; or Isopeptag and pilin-C; or SnoopTag and SnoopCatcher; or DogTag and SnoopTagJr; or SdyTag and SdyCatcher, or a combination of two or more of any of the listing pairs. It will be appreciated that the listed pairs specifically form covalent bonds with each other, and thus either member of the listed pairs may serve as “polypeptide tag” in accordance with detecting methods and kits of some embodiments herein, provided that the other pair member serves as the “counterpart polypeptide sequence.” Thus, for example, a Spytag may serve as a “polypeptide tag” whicle a SpyCatcher serves as a “counterpart polypeptide sequence,” or SpyCatcher may serve as a “polypeptide tag” while a SpyTag serves as a “counterpart polypeptide sequence. In the detecting method of some embodiments, the covalent polypeptide tag is fused to the polynucleotide via a HUH protein, SMCC linkage, or RepB replicase. In the detecting method of some embodiments, the identifier barcode further comprises a random oligonucleotide barcode or at least 5 nucleotides. In the detecting method of some embodiments, the identifier barcode further comprises a terminal single-stranded handle sequence. Each combinatorial barcode unit can comprise a terminal single-stranded complementary to the terminal handle sequence. The barcoding can comprises permitting the terminal single-stranded handle sequences to anneal to the terminal single-stranded complements, and ligating the terminal handle sequences to the terminal complements.

In the detecting method of some embodiments, the macromolecules are of a library of in vitro translated polypeptides, and each macromolecule comprises an “identifier barcode” comprising a polynucleotide comprising a coding sequence of the macromolecule such as an mRNA encoding the macromolecule. For example, the macromolecules can be translated in vitro from a polynucleotide encoding the macromolecule, in which a polypeptide tag is fused to the polynucleotide. The polynucleotide can further encode a counterpart polypeptide sequence that is part of the macromolecule, and specifically covalently binds to the polypeptide tag. The counterpart polypeptide sequence can be disposed in an N-terminal region of the macromolecule. As such, the polypeptide tag can co-translationally (or immediately following translation) form a covalent bond with the counterpart polypeptide sequence. For example, a 5′ portion of the polynucleotide can encode the counterpart polypeptide sequence so that an N-terminal portion of the macromolecule comprises the counterpart polypeptide sequence. Under these approaches, the macromolecule can be barcoded with an identifier barcode comprising the polynucleotide comprising the coding sequence of the macromolecule. Optionally, the polynucleotide of the identifier barcode may further comprise a random oligonucleotide barcode, for example a random oligonucleotide barcode of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 36, 37, 38, 39, 40, 41, 42, 43, 44, 45, 46, 47, 48, 49, or 50 nucleotides, including ranges between any two of the listed values, for example, 3-10, 3-20, 3-30, 3-50, 6-10, 6-20, 6-30, 6-50, 10-20, 10-30, 10-50, 20-30, or 20-50 nucleotides. Examples of suitable covalent polypeptide tag and counterpart polypeptide sequences include, but are not limited to, spilt CnaB proteins (See, e.g., Proschel et al., PLoS One 12(6):e0179740, which is hereby incorporated by reference in its entirety) such as a Spytag and SpyCatcher; or Isopeptag and pilin-C; or SnoopTag and SnoopCatcher; or DogTag and SnoopTagJr; or SdyTag and SdyCatcher. In some embodiments, the covalent polypeptide tag and counterpart polypeptide sequences comprise a split CnaB protein, or Spytag and SpyCatcher; or Isopeptag and pilin-C; or SnoopTag and SnoopCatcher; or DogTag and SnoopTagJr; or SdyTag and SdyCatcher, or a combination of two or more of the listed items. In some embodiments, the covalent polypeptide tag and counterpart polypeptide sequences comprise a Spytag and SpyCatcher; or Isopeptag and pilin-C; or SnoopTag and SnoopCatcher; or DogTag and SnoopTagJr; or SdyTag and SdyCatcher, or a combination of two or more of the listed items. It will be appreciated that the listed pairs specifically form covalent bonds with each other, and thus either member of the listed pairs may serve as a “polypeptide tag” in accordance with detecting methods and kits of some embodiments herein, provided that the other pair member serves as the “counterpart polypeptide sequence.” Thus, for example, a Spytag may serve as a “polypeptide tag” while a SpyCatcher serves as a “counterpart polypeptide sequence,” or SpyCatcher may serve as a “polypeptide tag” while a SpyTag serves as a “counterpart polypeptide sequence.

An mRNA molecule can be covalently bound to a macromolecule (or candidate interaction partner) using a polypeptide tag and counterpart polypeptide sequence that specifically form a covalent bond, such as a SpyTag-SpyCatcher mediated approach. A ribosome system can be used to express the macromolecule (or candidate interaction partner), which is fused to a polypeptide tag such as SpyTag. The mRNA can be ligated to an oligonucleotide that is coupled to a spy-catcher protein via a protein-oligonucleotide conjugation, such a HUH protein, SMCC linkage, or RepB replicase. The protein/mRNA linkage can be performed any number of reaction environments, for example, bacterial transcription/translation systems. Using this system, nascent translation of the mRNA produces a protein that, via a polypeptide tag and counterpart polypeptide sequence (such as SpyCatcher-SpyTag conjugation, or other systems) is covalently linked to its cognate mRNA. To facilitate fidelity of co-translational or immediately post-translational (nascent) RNA/nascent protein conjugation, the detecting method of some embodiments may comprise additional stalling sequences and/or translation in oil-in-water emulsions.

In some embodiments, the detecting method further comprises fusing polypeptide tags to a library of polynucleotides encoding a library of macromolecules. As used herein “fusing” (and variations of this root term) has its ordinary and customary meaning as would be understood by one of ordinary skill in the art in view of this disclosure. It refers to a forming covalent linkage between two molecules. Thus, a polynucleotide that is “fused” to a polypeptide tag is covalently bound to the polypeptide tag. The polypeptide tags can be fused to a library of polynucleotides by any of a number of forms of covalent attachment. For example, the polypeptide tag may further comprise a HUH protein, SMCC linkage, or RepB replicase which forms a covalent bond with a polynucleotide of the library. It will be appreciated that “HUH proteins,” “SMCC linkages,” and “RepB replicase” suitable for forming fusing to polynucleotides or oligonucleotides as described herein encompass full-length proteins, as well as covalent-bond-with-polynucleotide-forming fragments thereof. For example, the polynucleotide can be synthesized with a primary amine or thiol group, and an amine- or sulfhydryl-reactive crosslinker can covalently bind the polynucleotide to the polypeptide tag. In the detecting method of some embodiments, the polynucleotide can encode a counterpart polypeptide sequence that is part of the macromolecule, and specifically covalently binds to the polypeptide tag. Accordingly, when the coding sequence of the polynucleotide is translated, the macromolecule comprising the counterpart polypeptide sequence covalently binds to the polynucleotide comprising the coding sequence of the macromolecule.

### Exemplary Split-Pool Recognition of Interactions by Tag Extension (SPRITE) Embodiments

In some embodiments of the methods and compositions provided herein, one or more elements and/or steps of SPRITE can be employed. The systems, methods, compositions, and kits provided herein can, in some embodiments, be employed in concert with the compositions and methods described in Quinodoz et al. (“Higher-order inter-chromosomal hubs shape 3D genome organization in the nucleus.” Cell 174.3 (2018): 744-757), in Quinodoz et al (“RNA promotes the formation of spatial compartments in the nucleus.” Cell 184.23 (2021): 5775-5790) and in Quinodoz et al. (“SPRITE: a genome-wide method for mapping higher-order 3D interactions in the nucleus using combinatorial split-and-pool barcoding.” Nature protocols 17.1 (2022): 36-75) the contents of which can be herein incorporated by reference in their entirety. The systems, methods, compositions, and kits provided herein can, in some embodiments, be employed in concert with the systems, methods, compositions, and kits described in U.S. patent application Ser. No. 15/466,861, entitled “Methods for identifying macromolecule interactions”, filed Mar. 22, 2017, the content of which is incorporated herein by reference in its entirety. The systems, methods, compositions, and kits provided herein can, in some embodiments, be employed in concert with the systems, methods, compositions, and kits described in U.S. patent application Ser. No. 16/141,901, entitled, “Methods and systems for performing single cell analysis of molecules and molecular complexes”, filed Sep. 25, 2018, the content of which is incorporated herein by reference in its entirety.

As used herein the terms “associated” or “associated with” shall be given their ordinary meaning and can also refer to two or more species can be identifiable as being co-located at a point in time. An association can be an informatics association. For example, digital information regarding two or more species can be stored and can be used to determine that one or more of the species can be co-located at a point in time. An association can also be a physical association. In some embodiments, two or more associated species can be “tethered”, “attached”, or “immobilized” to one another or to a common solid or semisolid surface. An association may refer to covalent or non-covalent means for attaching labels to solid or semi-solid supports such as beads.

As used herein, the term “DNA” refers to deoxyribonucleic acid. DNA may be double stranded including both complementary strands, unless the DNA is shown to be or indicated to be single stranded (ss) DNA. As used herein, the term “RNA” refers to ribonucleic acid. RNA is a single stranded nucleic acid molecule, and as shown or indicated herein, may be a part of a double stranded molecule when complemented, for example, with copy DNA (cDNA) by reverse transcription. Nucleic acid molecules can comprise deoxyribonucleic acid molecules and/or ribonucleic acid molecules, and can be selected from the group comprising double-stranded DNA, single-stranded DNA, microRNA (miRNA), messenger RNA (mRNA), long non-coding RNA (lncRNA), ribosomal RNA (rRNA), transfer RNA (tRNA), small nuclear RNA (snRNA), small nucleolar RNA (snoRNA), Piwi-interacting RNA (piRNA), interfering RNA (siRNA), antisense RNA (aRNA), transfer messenger RNA (tmRNA), tRNA-derived small RNA (tsRNA), rDNA-derived small RNA (srRNA), ribozyme, viral RNA, double-stranded RNA, or any combination thereof.

As used herein, “suspension” refers to a liquid heterogeneous mixture. For example, a suspension may refer to a cell lysate having all of its cellular molecules in a liquid mixture. For example, a suspension may also include a cell lysate after homogenization, sonication, or chemical shearing. As used herein, “adding,” and like terms, can refer to the combination of two components together, no matter the order of the addition. For example, “adding” a nucleotide tag to a molecule is the same as “adding” a molecule to a nucleotide tag so long as the nucleotide tag and the molecule can be combined. As used herein, “distributing” and “sorting” can be used interchangeably to refer to the division of a whole quantity into a plurality of parts. For example, distributing or sorting a suspension involves the division of the whole suspension into multiple smaller suspensions. As used herein, “pooling” refers to collecting and mixing together a plurality of components. For example, pooling of suspensions includes mixing multiple suspensions into one larger, pooled suspension. As used herein, “shearing” or “fragmenting,” and like terms, can refer to chemical or mechanical means of separating or fragmenting a cell lysate. For example, shearing of chromatin (e.g., chromosomal DNA) may be carried out using mechanical means or chemical means. Non-limiting examples of mechanical shearing include sonication or homogenization. Non-limiting examples of chemical shearing, for example, of chromatin, include enzymatic fragmentation, using, for example DNase. Fragmenting can be enzymatic. For example, RNA can be fragmented (partially or fully) via enzymatic means, such as, for example, using RNase If.

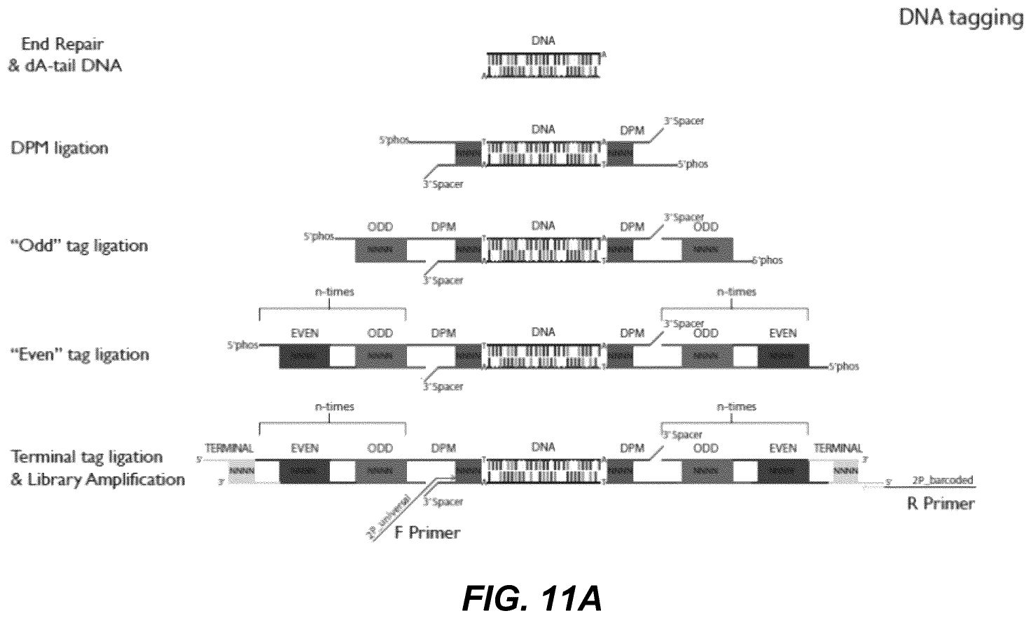

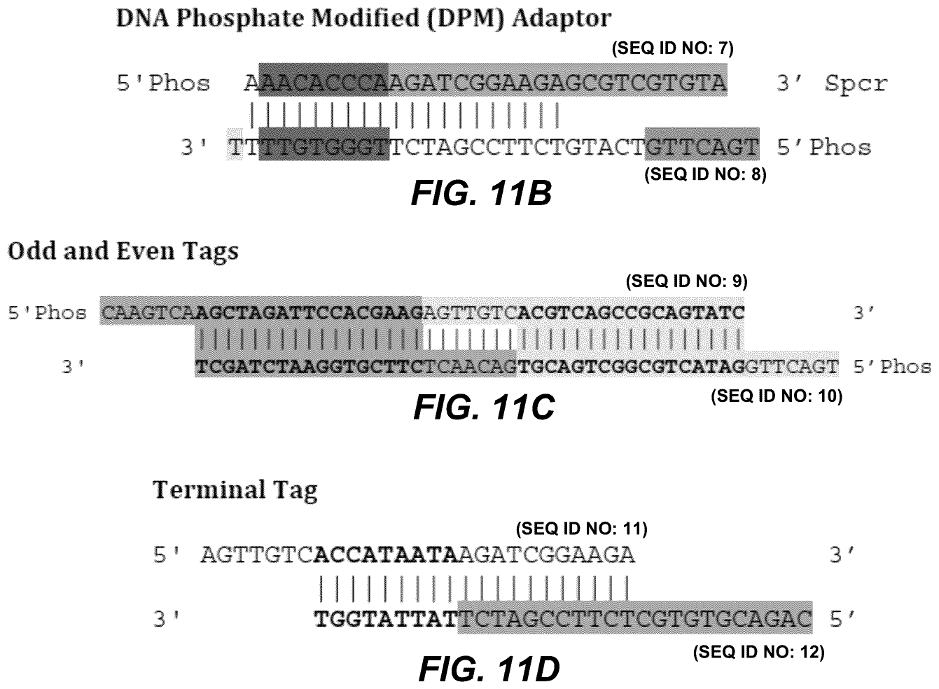

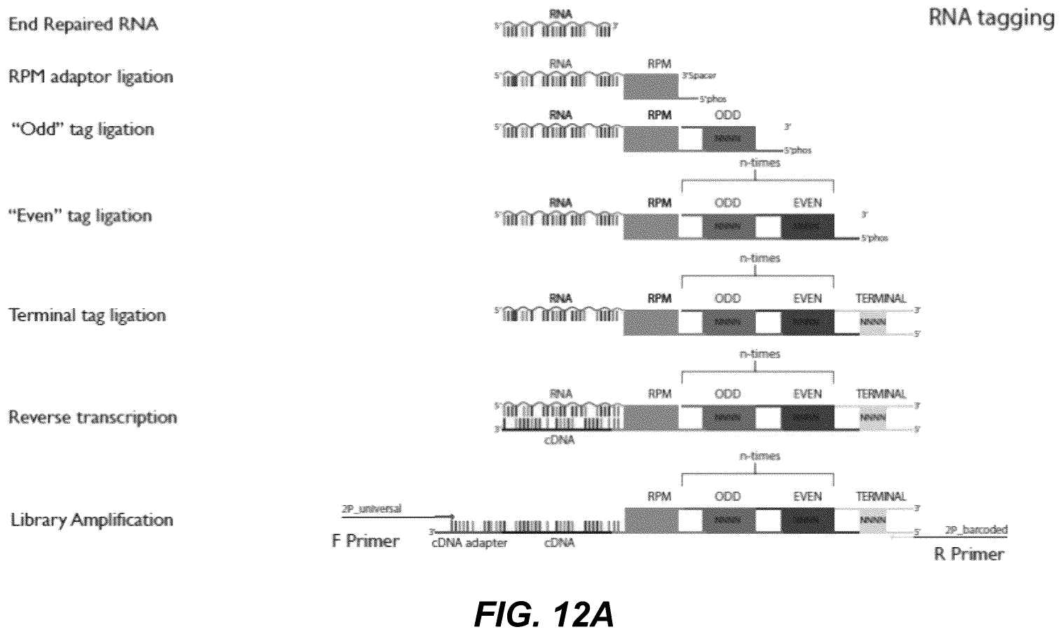

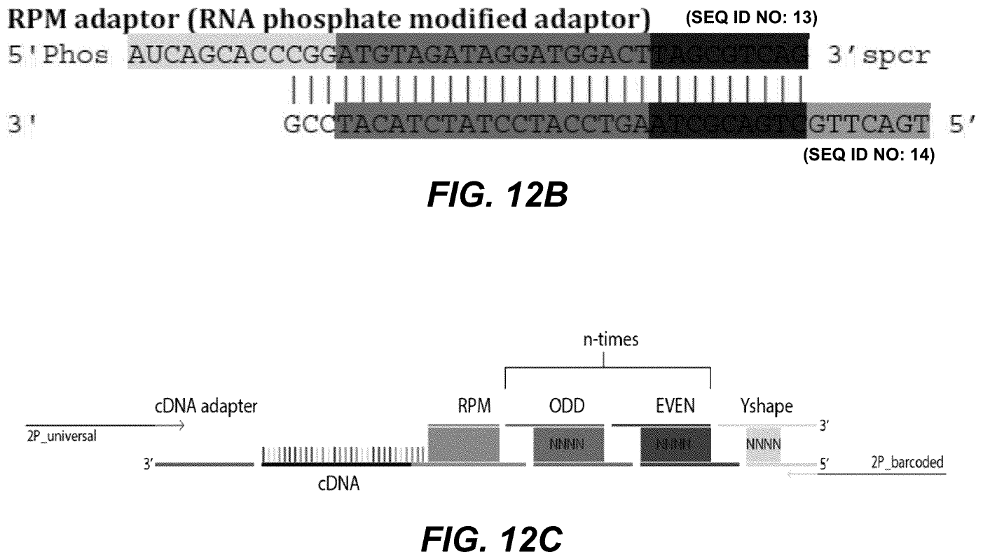

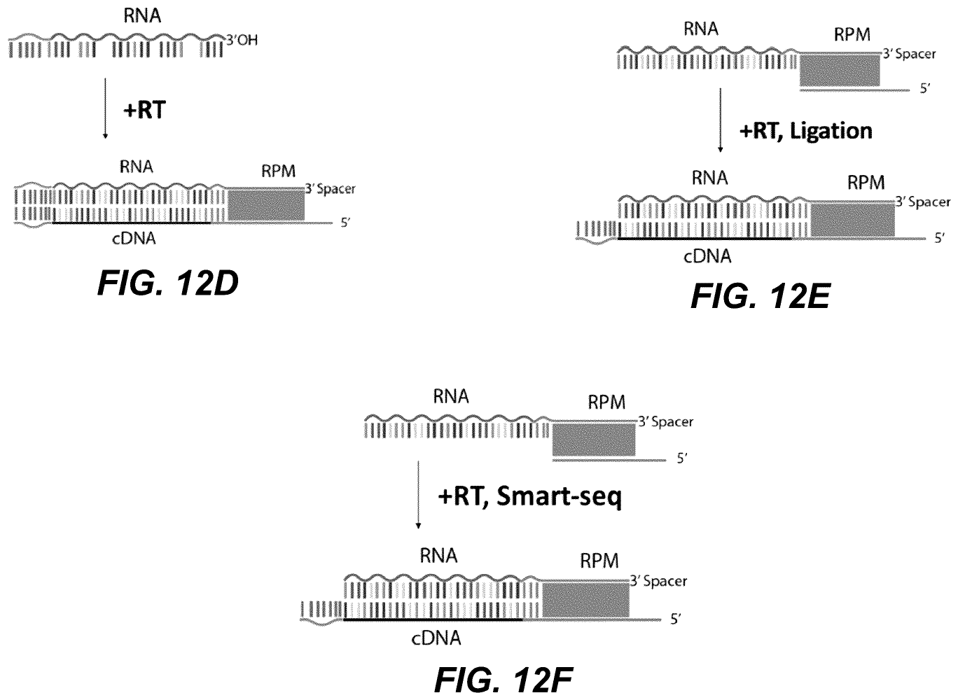

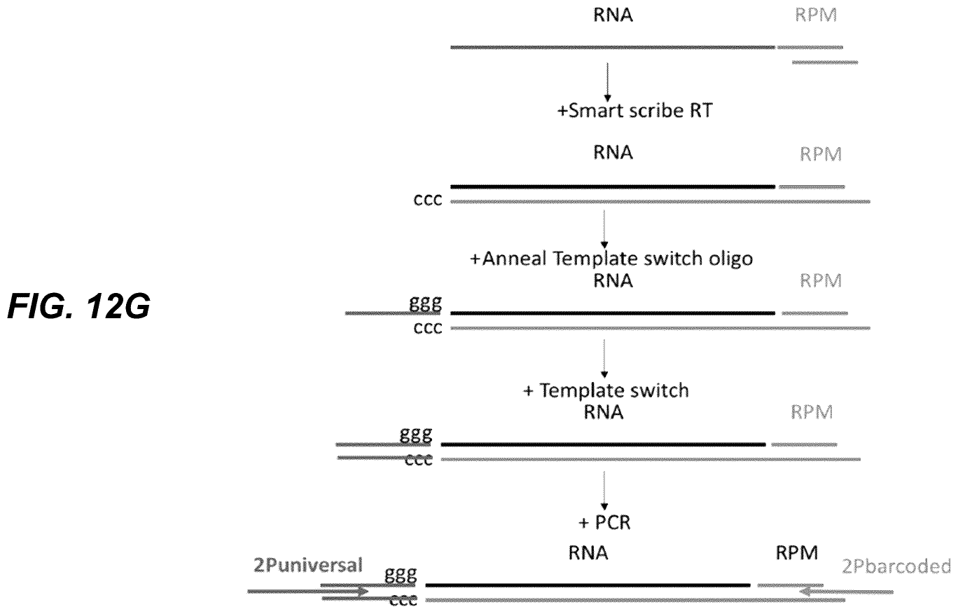

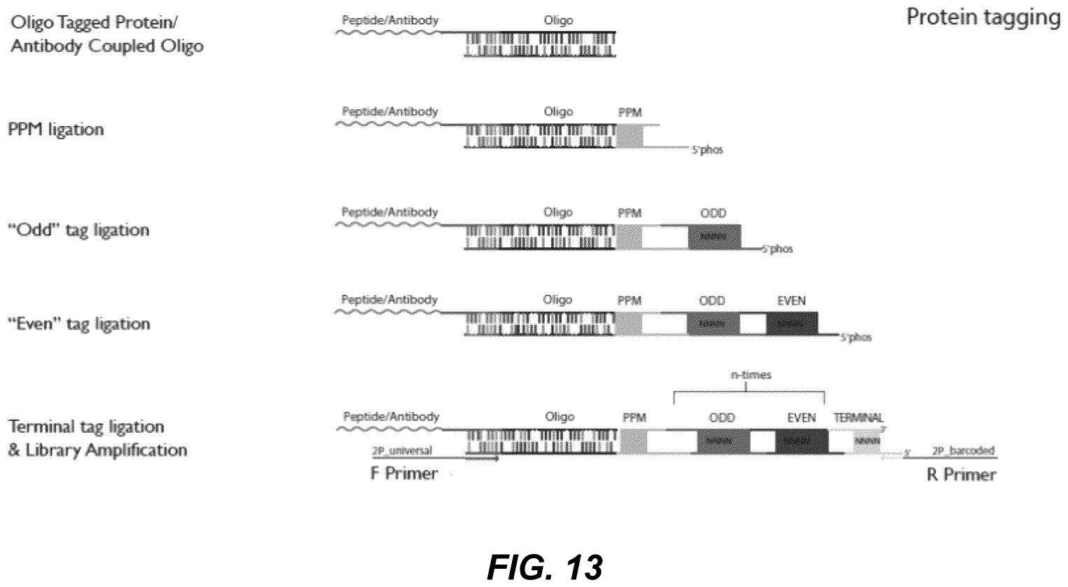

As used herein, the term “adaptor” refers to a molecule that may be coupled to a target molecule and enable or facilitate more effective nucleotide tagging (e.g., ligation), elongation, amplification, and/or sequencing of the target molecule. For example, DNA phosphate modified (DPM) adaptor according to embodiments disclosed herein and shown in FIG. 11A, is a molecule that couples to the 5′ and 3′ end of a DNA molecule allowing for the DNA molecule to be effectively ligated with a subsequent nucleotide tag. Another example of an adaptor is the RNA phosphate modified (RPM) adaptor according to embodiments disclosed herein and shown in FIG. 12A. The RPM adaptor couples to the 3′ end of an RNA molecule allowing for the RNA molecule to be effectively ligated with a subsequent nucleotide tag. In some embodiments disclosed herein, a protein phosphate modified (PPM) adaptor as shown in FIG. 13, is a molecule that couples to a target protein or to an antibody of a target protein, allowing for the protein to be effectively modified for subsequent nucleotide tagging. In some embodiments, the DPM, RPM, and/or PPM adaptor molecules may include a unique nucleotide sequence thereby also serving as a nucleotide tag. In addition to the tagging adaptors, a 5′ single stranded RNA (ssRNA) adaptor, for example, as shown in FIG. 12D, may be used, which ssRNA adaptor allows for the elongation of the RNA molecule for amplification and sequencing after 3′ nucleotide tagging of the RNA molecule.

As used herein, the terms “tagging” and “nucleotide tagging” can refer to the coupling of oligonucleotides to DNA, RNA, and/or protein molecules in order to label molecules that can be found to interact (directly or indirectly) in a complex. The tagging refers to the oligonucleotide label (tag) that identifies molecules that sort together thereby receiving the same tag. Additionally, coupling of oligonucleotides, according to embodiments disclosed herein, may also be used to enable molecules to be tagged. For example, as shown in FIG. 13, a protein or antibody may be coupled with an oligonucleotide in order for the protein or antibody molecule to subsequently receive (e.g., ligate) a nucleotide tag or receive a protein phosphate modified (PPM) adaptor that is capable of ligating a nucleotide tag. The coupling of oligonucleotides to proteins or antibodies is shown herein, but is also described in Los et al., “HaloTag: a novel protein-labeling technology for cell imaging and protein analysis, ACS Chem Biol., 2008, 3:373-382; Singh et al., “Genetically Encoded Multispectral Labeling of Proteins with Polyfluorophores on a DNA Backbone,” J. Am. Chem. Soc., 2013, 16:6184-6191; Blackstock et al., “Halo-Tag Mediated Self-Labeing of Fluorescent Proteins to Molecular Beacons for Nucleic Acid Detection,” Chem. Commun., 2014, 50: 1375-13738; Kozlov et al., “Efficient Strategies for the Conjugation of Oligonucleotides to Antibodies Enabling Highly Sensitive Protein Detection,” Biopolymers, 2004, 73:621; and Solulink, “Antibody-Oligonucleotide Conjugate Preparation,” Solulink.com, 4 pages, the entire contents of all of which can be incorporated herein by reference.

As used herein, “combinatorial barcode” has its customary and ordinary meaning as would be understood by one of ordinary skill in the art in view of this disclosure. It refers to a type of barcode that comprises multiple “combinatorial barcode units,” which together yield the combinatorial barcode. For example, each combinatorial barcode unit (e.g., Odd Tag, Even Tag) can comprise an oligonucleotide subunit, and the sequence of the oligonucleotide subunit can provide identification information for the combinatorial barcode unit. By way of example, a combinatorial barcode unit may comprise, consist essentially of, or consist of an oligonucleotide of at least 3, 4, 5, 6, 7, 8, 9, 10, 11, 12, 13, 14, 15, 16, 17, 18, 19, or 20 nucleotides in length, including ranges between any two of the listed values, for example, 3-8, 3-12, 3-16, or 3-20, 4-8, 4-12, 4-16, 4-20, 6-8, 6-12, 6-16, 6-20, 10-12, 10-16, or 10-20 nucleotides. The number of different combinatorial barcode units, and the length of the combinatorial barcode may depend on the scale of the detecting method or kit. For example, if there are at least “m” different partitions, the there may be at least “m” different combinatorial barcode units, so that each partition may be associated with a different combinatorial barcode unit. A combinatorial barcode may comprise at least 2 combinatorial barcode units, for example, at least 2, 3, 4, 5, 6, 7, 8, 9, 10, 11, 12, 13, 14, 15, 16, 17, 18, 19, or 20, including ranges between any two of the listed values, for example, 2-8, 2-12, 2-16, 2-20, 3-8, 3-12, 3-16, 3-20, 4-8, 4-12, 4-16, 4-20, 6-8, 6-12, 6-16, 6-20, 10-12, 10-16, or 10-20 combinatorial barcode units.

As used herein, “split-and-pool barcoding” has its customary and ordinary meaning as would be understood by one of ordinary skill in the art in view of this disclosure. It refers to barcoding in which a composition comprising molecules is split into two or more partitions that are separate from each other. Then, the composition of each partition is barcoded so that molecules in the same partition are barcoded with the same barcode, but molecules in different partitions are barcoded with different barcodes from each other. After the barcoding, the contents of the partitions can be pooled to form a composition. The process can be repeated on this composition, so that multiple iterations of splitting, barcoding, and pooling are performed. The “partitions” refer to spaces that are in fluid isolation from each other, so that the contents of the different partitions do not mix while they are in the partitions. For example, the partitions can be separated by one or more solid barriers. Examples of partitions include, but are not limited to, wells of a multi-well plate (e.g., 96-well plate), containers such as microcentrifuge tubes, chambers of a fluid device, and the like. After multiple iterations of split-and-pool barcoding, the molecules (e.g., nucleic acids) will each comprise a combination of combinatorial barcode units. These combinations may be referred to as “combinatorial barcodes” (and accordingly, the barcoding to produce the combinatorial barcodes may be referred to as “combinatorial barcoding.”).

Distribution or sorting of the suspension into the lysate suspensions may be performed using any suitable approach. As described in the examples disclosed herein, distribution of the suspension may be accomplished using a 96-well plate, thereby resulting in 96 suspensions and 96 unique nucleotide tags. The number of suspensions is not limited to a minimum or maximum. As is understood by the skilled person, an increase in the number of suspensions will increase the probability of sorting non-interacting molecules apart from each other. As used herein, a “well” refers to the well of a 96-plate, however, any number of wells or plates may be used. A well may also refer to the well of a tube or any similar vessel capable of holding the sorted lysate suspension separate from other sorted lysate suspensions. For example, a well may also include a flat surface.

To each of the distributed lysate suspensions, a unique nucleotide tag may be added. As used herein, “unique” means different from any other. As noted above in the definition of adding, either the unique nucleotide tag can be added to its respective distributed lysate suspension, or the distributed suspension may be added to a well containing its respective unique nucleotide tag. For example, in a 96-well set up, a plurality of lysate suspensions would refer to 96 suspensions receiving one of 96 different nucleotide tags. Each unique nucleotide tag is capable of tagging the DNA, RNA, and/or protein molecules in the lysate suspension. In some embodiments, the nucleotide tagging is facilitated by an adaptor molecule, such as the DPM, RPM, or PPM disclosed herein. In some embodiments, the nucleotide tagging of a protein molecule includes expressing a modified protein of interest in a cell, in which the expressed modified protein is capable of being coupled to an oligonucleotide. The oligonucleotide directly coupled to the protein may serve as a nucleotide tag for identification. In some embodiments, the oligonucleotide coupled to the protein may be ligated with subsequent nucleotide tags. In some embodiments, an antibody that binds to a target protein may be modified with an oligonucleotide. The antibody coupled oligonucleotide enables the protein to be labeled which may serve as a nucleotide tag for identification. In some embodiments, the oligonucleotide coupled to the antibody may be ligated with subsequent nucleotide tags. In some embodiments, an antibody modified with an oligonucleotide is incubated with the cell lysate prior to nucleotide tagging.

After a unique first nucleotide tag is coupled or ligated to each of the plurality of lysate suspensions, the lysate suspensions may be pooled, thereby forming a first tagged pool. In some embodiments, the first nucleotide tag may be any suitable oligonucleotide that is capable of being sequenced. In some embodiments, the first nucleotide tag is added to any one sorted lysate suspension is capable of binding to all DNA, RNA and/or protein molecules. In some embodiments, the first nucleotide tag is capable of ligating to all DNA, RNA, and/or protein molecules in the lysate suspension that have been modified with a DPM, RPM, or PPM adaptor as disclosed herein. This first nucleotide tag may be referred to as an “Odd” nucleotide tag as shown in FIGS. 11A, FIG. 12A, and FIG. 13. In some embodiments, depending on the approach and strategy used to target a complex, one distribution of the suspension may be adequate for identifying true interactions of molecules. Accordingly, the nucleotide tags in the first tagged pool may be amplified and subsequently sequenced for analysis. In some embodiments, the probability that non-interacting molecules will receive all of the same nucleotide tags decreases exponentially with each additional round of tagging and sorting. Accordingly, in some embodiments, the first tagged pool is distributed into a plurality of tagged pool suspensions. In some embodiments, the first tagged pool may be mixed thoroughly prior to redistribution to ensure separation of non-interacting complexes. To each of the distributed plurality of tagged pool suspensions, a unique second nucleotide tag may be added (or each of the plurality of tagged pool suspensions may be added to its respective unique second nucleotide tag). In some embodiments, all of the second nucleotide tags can be capable of ligating to any of the previously ligated first nucleotide tags. This second nucleotide tag is referred to as an “Even” nucleotide tag as shown in FIGS. 11A, FIG. 12A, and FIG. 13.

After a unique second nucleotide tag is coupled or ligated to each tagged pool suspension, the tagged pool suspensions may again be pooled forming a second tagged pool. In some embodiments, the nucleotide tags in the second tagged pool may be amplified and sequenced, or redistributed for another round of tagging. The pooling, distributing (sorting), and tagging may continue indefinitely so long as the integrity of the samples is maintained, and unique nucleotide tags remain available. In some embodiments, the second tagged pool is redistributed into a plurality of tagged re-pooled suspensions for a third nucleotide tagging in which the third nucleotide tag ligates to any of the second nucleotide tags. The third nucleotide tag may be referred to as an “Odd” tag as it can ligate to the previous “Even” tag. Nucleotide tagging may continue indefinitely so long as the previous tag is capable of ligating the subsequent tag. An example of this is the Odd to Even to Odd tagging as shown in FIG. 11A and FIG. 11C. The ligation sequences of these tags alternate to ensure ligation fidelity. The third nucleotide tagging may be followed again by pooling of the tagged re-pooled suspensions to form a third tagged pool which may be amplified for sequencing. In some embodiments, the third tagged pool may be distributed into a plurality of tagged thrice pooled suspensions for a fourth nucleotide tagging in which the fourth nucleotide tag ligates to any of the previously ligated third nucleotide tags. The fourth nucleotide tagging may be followed again by pooling of the tagged thrice pooled suspensions to form a fourth tagged pool which may be amplified for sequencing. In some embodiments, the fourth tagged pool may be distributed into a plurality of tagged 4× pooled suspensions for a fifth nucleotide tagging. In some embodiments, after the first nucleotide tagging, the pooling, distributing, and tagging may be carried out (n) number of times, such that the DNA, RNA, and/or protein molecules in the suspension receive (n)+1 number of nucleotide tags. In some embodiments, after the desired number of sorting and tagging has been performed, the plurality of tagged (n)x pooled suspensions can be pooled into a final pool and the tagged molecules in the final pool can be amplified for sequencing. In some embodiments, after the last nucleotide tag is added, the final pool may be redistributed again into a plurality of tagged final pool suspensions for the addition of a Terminal nucleotide tag. As shown in FIG. 11D, a Terminal tag may provide an additional unique sequence and may also provide a primer site for amplification. In some embodiments disclosed herein, the tagged final pool is first amplified to make a library of amplified tags as disclosed herein. Amplified tags can be then sequenced using next generation sequencing as disclosed.

### Exemplary RD-SPRITE Embodiments

In some embodiments of the methods and compositions provided herein, one or more elements and/or steps of RD-SPRITE can be employed. In some embodiments, RD-SPRITE can improve detection of lower abundance RNAs by increasing yield through one or more of the following adaptations. (i) The RNA ligation efficiency can be increased by utilizing a higher concentration of RPM, corresponding to −2000 molar excess during RNA ligation. (ii) Adaptor dimers that can be formed through residual purification on our magnetic beads can lead to reduced efficiency because they can preferentially amplify and preclude amplification of tagged RNAs. To reduce the number of adaptor dimers in library generation, an exonuclease digestion of excess reverse transcription (RT) primer can be introduced that dramatically reduces the presence of the RT primer. (iii) Reverse transcription can be used to add the barcode to the RNA molecule, yet when RT is performed on crosslinked material it will not efficiently reverse transcribe the entire RNA (because crosslinked proteins will act to sterically preclude RT). To address this, a short RT in crosslinked samples can be performed followed by a second RT reaction after reverse crosslinking to copy the remainder of the RNA fragment. (iv) Because cDNA is single-stranded, a second adaptor needs to ligated in some embodiments to enable PCR amplification. The efficiency of this reaction can be important for ensuring that a user detects each RNA molecule. cDNA ligation efficiency can be improved by introducing a modified “splint” ligation. Specifically, a double stranded “splint” adaptor containing the Readl Illumina priming region and a random 6-mer overhang is ligated to the 3′ end of the cDNA at high efficiency by performing a double stranded DNA ligation. This process can be more efficient than the single stranded DNA-DNA ligation previously utilized (Quinodoz et al., 2018). (v) Finally, nucleic acid purification performed after reverse crosslinking can lead to major loss of complexity because a percentage of the unique molecules is lost during each cleanup. In the initial RNA-DNA SPRITE protocol there can be several column (or bead) purifications utilized to remove enzymes and enable the next enzymatic reaction. These cleanups can be reduced by introducing biotin modifications into the DPM and RPM adaptors that enable binding to streptavidin beads and for all subsequent molecular biology steps to occur on the same beads. Together, these improvements can enable a dramatic improvement of our overall RNA recovery and enables generation of high complexity RNA/DNA structure maps.

Crosslinking, Lysis, Sonication, and Chromatin Digestion

Cells can be lifted using trypsinization and can be crosslinked in suspension at room temperature with 2 mM disuccinimidyl glutarate (DSG) for 45 minutes followed by 3% Formaldehyde for 10 minutes to preserve RNA and DNA interactions in situ. After crosslinking, the formaldehyde crosslinker can be quenched with addition of 2.5M Glycine for final concentration of for 5 minutes, cells can be spun down, and resuspended in 1×PBS+0.5% RNase Free BSA (AmericanBio AB01243-00050) over three washes, 1×PBS+0.5% RNase-Free BSA can be removed, and flash frozen at −80 C for storage. RNase Free BSA can be important to avoid RNA degradation. RNase Inhibitor (1:40, NEB Murine RNase Inhibitor or Thermofisher Ribolock) can be also added to all lysis buffers and subsequent steps to avoid RNA degradation. After lysis, cells can be sonicated at 4-5W of power for 1 minute (pulses 0.7 s on, 3.3 s off) using the Branson Sonicator and chromatin can be fragmented using DNase digestion to obtain DNA of approximately −150 bp-lkb in length.

Estimating Molarity

After DNase digestion, crosslinks can be reversed on approximately 10 μL of lysate in 82 μL of 1× Proteinase K Buffer (20 mM Tris pH 7.5, 100 mM NaCl, 10 mM EDTA, 10 mM EGTA, 0.5% Triton-X, 0.2% SDS) with 8 μL Proteinase K (NEB) at 65° C. for 1 hour. RNA and DNA can be purified using Zymo RNA Clean and Concentrate columns per the manufacturer's specifications (>17nt protocol) with minor adaptations, such as binding twice to the column with 2× volume RNA Binding Buffer combined with by 1× volume 100% EtOH to improve yield. Molarities of the RNA and DNA can be calculated by measuring the RNA and DNA concentration using the Qubit Fluorometer (HS RNA kit, HS dsDNA kit) and the average RNA and DNA sizes can be estimated using the RNA High Sensitivity Tapestation and Agilent Bioanalyzer (High Sensitivity DNA kit).

DNA End Repair and dA-Tailing

The DNA ends can then be repaired to enable ligation of tags to each molecule. Specifically, blunt end and phosphorylate the 5′ ends of double-stranded DNA using two enzymes in some embodiments. First, the NEBNext End Repair Enzyme cocktail (E6050L; containing T4 DNA Polymerase and T4 PNK) and 1×NEBNext End Repair Reaction Buffer can be added to beads and incubated at 20° C. for 1 hour, and inactivated and buffer exchanged as specified above. DNA can be then dA-tailed using the Klenow fragment (5′-3′ exo-, NEBNext dA-tailing Module; E6053L) at 37° C. for 1 hour, and inactivated and buffer exchanged as specified above. To prevent degradation of RNA, each enzymatic step can be performed with the addition of 1:40 NEB Murine RNase Inhibitor or Thermofisher Ribolock.

Ligation of the DNA Phosphate Modified (“DPM”) Tag

After end repair and dA-tailing of DNA, a pooled ligation can be performed with “DNA Phosphate Modified” (DPM) tag that contains certain modifications. Specifically, (i) incorporating a phosphothiorate modification into the DPM adaptor to prevent its enzymatic digestion by Exo1 in subsequent RNA steps and (ii) integrated an internal biotin modification to facilitate an on-bead library preparation post reverse-crosslinking. The DPM adaptor can also contain a 5′ phosphorylated sticky end overhang to ligate tags during split-pool barcoding. DPM Ligation can be performed using 11 μL of 4.5 μM DPM adaptor in a 250 μL reaction using Instant Sticky End Mastermix (NEB) at 20° C. for 30 minutes with shaking. All ligations can be supplemented with 1:40 RNase inhibitor (ThermoFisher Ribolock or NEB Murine RNase Inhibitor) to prevent RNA degradation. Because T4 DNA Ligase only ligates to double-stranded DNA, the unique DPM sequence enables accurate identification of DNA molecules after sequencing.

Ligation of the RNA Phosphate Modified (“RPM”) Tag

To map RNA and DNA interactions simultaneously, an RNA adaptor is ligated to RNA that contains the same 7nt 5′ phosphorylated sticky-end overhang as the DPM adaptor to ligate tags to both RNA and DNA during split-pool barcoding. To do this, the 3′ end of RNA is first modified to ensure that they all have a 3′ OH that is compatible for ligation. Specifically, RNA overhangs can be repaired with T4 Polynucleotide Kinase (NEB) with no ATP at 37° C. for 20 min. RNA can be subsequently ligated with a “RNA Phosphate Modified” (RPM) adaptor using High Concentration T4 RNA Ligase I (Shishkin et al., n15). Briefly, beads can be resuspended in a solution consisting of 30 μL 100% DMSO, 154 μL H₂O, and 20 μL of 20 μM RPM adaptor, heated at 65° C. for 2 minutes to denature secondary structure of RNA and the RPM adaptor, then immediately put on ice. An RNA ligation master mix can be added on top of this mixture consisting of: 40 μL 10×NEB T4 RNA Ligase Buffer, 4 μL 100 mM ATP (NEB), 120 μL 50% PEG 8000 (NEB), 20 μL Ultra Pure H₂O, 6 μL Ribolock RNase Inhibitor, 7 μL NEB T4 RNA Ligase, High Concentration (NEB, M0437M) for 24° C. for with shaking 1 hour 15 minutes. Because T4 RNA Ligase 1 only ligates to single-stranded RNA, the unique RPM sequence enables accurate identification of RNA and DNA molecules after sequencing. After RPM ligation, RNA can be converted to cDNA using Superscript III at 42° C. for 1 hour using the “RPM bottom” RT primer that contains an internal biotin to facilitate on-bead library construction (as above) and a 5′ end sticky end to ligate tags during SPRITE. Excess primer can be digested with Exonuclease 1 at 42° C. for 10⁻¹⁵ min. All ligations can be supplemented with 1:40 RNase inhibitor (ThermoFisher Ribolock or NEB Murine RNase Inhibitor) to prevent RNA degradation.

Split-and-Pool Barcoding to Identify RNA and DNA Interactions

The beads can be then repeatedly split-and-pool ligated over four rounds with a set of “Odd,” “Even” and “Terminal” tags (see SPRITE Tag Design above and Quinodoz et al., 2018). Both DPM and RPM contain the same 7 nucleotide sticky end that will ligate to all subsequent split-pool barcoding rounds. All split-pool ligation steps can be performed for 45 min to 1 hour at 20° C. Specifically, each well contained the following: 2.4 μL well-specific 0.45 μM SPRITE tag (IDT), 6.4 μL custom SPRITE ligation master mix, 5.6 μL SPRITE wash buffer (described above), and 5.6 μL Ultra-Pure H₂O. For all SPRITE ligations, a custom SPRITE ligation master mix (3.125× concentrated) can be made by combining 1600 μL of 2× Instant Sticky End Mastermix (NEB; M0370), 600 μL of 1,2-Propanediol (Sigma-Aldrich; 398039), and 1000 μL of 5×NEBNext Quick Ligation Reaction Buffer (NEB; B6058S). All ligations can be supplemented with 1:40 RNase inhibitor (ThermoFisher Ribolock or NEB Murine RNase Inhibitor) to prevent RNA degradation.

Reverse Crosslinking

After multiple rounds of SPRITE split-and-pool barcoding, the tagged RNA and DNA molecules can be eluted from NHS beads by reverse crosslinking overnight (˜12-13 hours) at 50° C. in NLS Elution Buffer (20 mM Tris-HCl pH 7.5, 10 mM EDTA, 2% N-Lauroylsarcosine, 50 mM NaCl) with added 5M NaCl to 288 mM NaCl Final combined with 5 μL Proteinase K (NEB).

Post Reverse-Crosslinking Library Preparation

AEBSF (Gold Biotechnology CAS #30827-99-7) can be added to the Proteinase K (NEB Proteinase K #P8107S; ProK) reactions to inactive the ProK prior to coupling to streptavidin beads. Biotinylated barcoded RNA and DNA can be bound to Dynabeads MyOne Streptavidin C1 beads (ThermoFisher #65001). To improve recovery, the supernatant can be bound again to 20 μL of streptavidin beads and combined with the first capture. Beads can be washed in 1×PBS+RNase inhibitor and then resuspended in 1× First Strand buffer to prevent any melting of the RNA:cDNA hybrid. Beads can be pre-incubated at 40 C for 2 min to prevent any sticky barcodes from annealing and extending prior to adding the RT enzyme. A second reverse transcription can be performed by adding Superscript III (Invitrogen #18080051) (without RT primer) to extend the cDNA through the can be as which can be previously crosslinked. The second RT ensures that cDNA recovery can be maximal, particularly if RT terminated at a crosslinked site prior to reverse crosslinking. After generating cDNA, the RNA can be degraded by addition of RNaseH (NEB #M0297) and RNase cocktail (Invitrogen #AM2288), and the 3′ end of the resulting cDNA can be ligated to attach an dsDNA oligo containing library amplification sequences for subsequent amplification.

Some embodiments comprise performing cDNA (ssDNA) to ssDNA primer ligation which relies on the two single stranded sequences coming together for conversion to a product that can then be amplified for library preparation. To improve the efficiency of cDNA molecules ligated with the Readl Illumina priming sequence, in some embodiments a “splint” ligation can be performed, which involves a chimeric ssDNA-dsDNA adaptor that contains a random 6-mer that anneals to the 3′ end of the cDNA and brings the 5′ phosphorylated end of the cDNA adaptor directly together with the cDNA via annealing. This ligation can be performed with 1× Instant Sticky End Master Mix (NEB #M0370) at 20° C. for 1 hour. This greatly improves the cDNA tagging and overall RNA yield in some embodiments.

Libraries can be amplified using 2×Q5 Hot-Start Mastermix (NEB #M0494) with primers that add the indexed full Illumina adaptor sequences. After amplification, the libraries can be cleaned up using 0.8×SPRI (AMPure XP) and then gel cut using the Zymo Gel Extraction Kit selecting for sizes between 280 bp-1.3 kb.

Annealing of Adaptors

A double-stranded DPM oligo and 2P universal “splint” oligo can generated by annealing the complementary top and bottom strands at equimolar concentrations. dsDNA SPRITE oligos can be annealed in 1× Annealing Buffer (0.2 M LiCl₂, 10 mM Tris-HCl pH 7.5) by heating to 95° C. and then slowly cooling to room temperature (−1° C. every 10 s) using a thermocycler.

TABLE 1
Primers Used for RPM, DPM, and Splint Ligation

| SEQ | | |
|---|---|---|
| ID | | |
| NO: | Name | Sequence |
| 1 | RPM top | /5Phos/rArUrCrArGrCrACTTAGCG |
| | | TCAG/3SpC3/ |
| 2 | RPM | /5Phos/TGACTTGC/iBiodT/ |
| | bottom | GACGCTAAGTGCTGAT |
| | (internal | |
| | biotin) | |
| 3 | DPM | /5Phos/ |
| | Phosphor- | AAGACCACCAGATCGGAAGAGCGTCGTG*T* |
| | othioate | A*G*G*/32MOErG/ |
| | top | |
| 4 | DPM | /5Phos/TGACTTGTCATGTCT/iBioT/ |
| | bottom | CCGATCTGGTGGTCTTT |
| | (internal | |
| | biotin) | |
| 5 | 2Puni | TACACGACGCTCTTCCGATCT NNNNNN/3SpC3/ |
| | splint | |
| | top | |
| 6 | 2Puni | /5Phos/AGA TCG GAA GAG CGT CGT GTA/ |
| | splint | 3SpC3/ |
| | bottom | |

*Denotes Phosphorothioate bonds

### EXAMPLES

Some aspects of the embodiments discussed above are disclosed in further detail in the following examples, which are not in any way intended to limit the scope of the present disclosure.

Experiments were performed using the methods and compositions provided herein, as described below, and also as noted in Quinodoz et al (2018) and Quinodoz et al (2021).

### Materials and Methods

ChIP-DIP

Cell Culture (Mouse Embryonic Stem Cells, Human K562 Cells)

(i) All mouse ES cell lines were grown at 37 C under 7% CO2 on plates coated with 0.2% gelatin (Sigma, G1393-100ML) and 1.75 mg/mL laminin (Life Technologies Corporation, #23017015) in serum-free 2i/LIF media composed as follows: 1:1 mix of DMEM/F-12 (GIBCO) and Neurobasal (GIBCO) supplemented with 1×N2 (GIBCO), 0.5×B-27 (GIBCO 17504-044), 2 mg/mL bovine insulin (Sigma), 1.37 mg/mL progesterone (Sigma), 5 mg/mL BSA Fraction V (GIBCO), 0.1 mM 2-mercaptoethanol (Sigma), 5 ng/mL murine LIF (GlobalStem), 0.125 mM PD0325901 (SelleckChem) and 0.375 mM CHIR99021 (SelleckChem). 2i inhibitors were added fresh with each medium change. Fresh medium was replaced every 24-48 hours depending on culture density, and cells were passaged every 72 hours using 0.025% Trypsin (Life Technologies) supplemented with 1 mM EDTA and chicken serum (1/100 diluted; Sigma), rinsing dissociated cells from the plates with DMEM/F12 containing 0.038% BSA Fraction V. (ii) K562 cells (ATCC, CCL-243) were purchased from ATCC and cultured under standard conditions outlines on the ATCC website.

Crosslinking (1% Formaldehyde)

(i) Mouse ES cells were trypsinized by adding 5 mL of TVP (1 mM EDTA, 0.025% Trypsin, 1% Sigma Chicken Serum; pre-warmed at 37 C) to each 15 cm plate, then rocked gently for 3-4 min until cells start to detach from the plate. Afterward, 25 mL of wash solution (DMEM/F-12 supplemented with 0.03% GIBCO BSA Fraction V, pre-warmed at 37 C) was added to each plate to inactivate the trypsin. Cells were lifted and transferred into a 15 mL or 50 mL conical tube, pelleted at 330 g for 3 min, and then washed in 4 mL of 1×PBS per 10 million cells. (ii) K562 cells were harvested by transferring the cell suspension to a 50 mL conical tube, pelleting at 330 g for 3 min and washing with 4 mL of 1×PBS per 10 million cells.

During all following crosslinking and wash steps, volumes were maintained at 4 mL of buffer or crosslinking solution per 10 million cells. After pelleting in 1×PBS, cells were first briefly resuspended in 1 mL of 1×PBS per 10 million cells and pipetted to disrupt clumps of cells. Next, cells were crosslinked in suspension in a final volume of 4 mL of 1% formaldehyde (FA Ampules, Pierce) diluted in 1×PBS per 10 million cells and rocked gently for 10 min at room temperature. Formaldehyde was immediately quenched with addition of 200 ml of 2.5 M glycine per 1 mL of 1% FA solution and incubated for 5 min at room temperature. Cells were pelleted, formaldehyde was removed, and cells were washed three times with 0.5% BSA in 1×PBS that was kept at 4 C. Aliquots of 10 million cells were allocated into 1.7 mL tubes and pelleted. Supernatant was removed and cells were flash frozen in liquid nitrogen and stored in 80 C until lysis.

Nuclear Isolation

Crosslinked cell pellets (10 million cells) were lysed using the following nuclear isolation procedure: cells were incubated in 0.7 mL of Nuclear Isolation Buffer 1 (50 mM HEPES pH 7.4, 1 mM EDTA pH 8.0, 1 mM EGTA pH 8.0, 140 mM NaCl, 0.25% Triton-X, 0.5% NP-40, 10% Glycerol, 1×PIC) for 10 min on ice. Cells were pelleted at 850 g for 10 min at 4 C. Supernatant was removed, 0.7 mL of Lysis Buffer 2 (50 mM HEPES pH 7.4, 1.5 mM EDTA, 1.5 mM EGTA, 200 mM NaCl, 1×PIC) was added and incubated for 10 min on ice. Nuclei were obtained after pelleting and supernatant was removed (as above), and 550 uL of Lysis Buffer 3 (50 mM HEPES pH 7.4, 1.5 mM EDTA, 1.5 mM EGTA, 100 mM NaCl, 0.1% sodium deoxycholate, 0.5% NLS, 1×PIC) was added and incubated for 10 min on ice prior to sonication.

Chromatin Fragmentation and Fragment Size Analysis

Chromatin was fragmented via sonication of the nuclear pellet using a Branson needle-tip sonicator (3 mm diameter (⅛″ Doublestep), Branson Ultrasonics 101-148-063) at 4 C for a total of 2.5 min at 4-5 W (pulses of 0.7 son, followed by 3.3 s off). To check the resulting DNA size distribution, a small aliquot of 20 uL of sonicated lysate was then added to 80 uL of Proteinase K buffer ((20 mM Tris pH 7.5, 100 mM NaCl, 10 mM EDTA, 10 mM EGTA, 0.5% Triton-X, 0.2% SDS) and reverse crosslinked at 80 C for 30 minutes. DNA was isolated using Zymo DNA Clean and Concentrator columns and eluted in water. 10 uL of purified DNA was then run for 10 minutes on a 1% e-gel (Invitrogen™ E-Gel™ EX Agarose Gels, 1%, Cat. No. G402021). Fragments were found to be 150-700 bp with an average size of roughly 350 bp. The remaining chromatin prep was stored at 4 C overnight to be used for the immunoprecipitation the next day.

Antibody ID Oligo Design

Antibody ID oligos were designed and ordered from IDT. The structure from 3′ to are: a 3′ biotin (/3Bio/), spacer sequence, Illumina's 2P Universal sequence, 9mer Antibody ID sequence, 8mer UMI, Odd sticky end.

The 5′ end of the oligo tag has a modified phosphate group and a complementary sequence that allows for ligation to the sticky-end overhang of the first set of Odd adaptors. The 3′ end of the oligo tag has a biotin group that allows for binding to free streptavidin, which is then used to subsequently couple to biotinylated Protein G beads.

The oligo tag also contains the following: (i) a partial sequence that is complementary to the universal Readl Illumina primer, which is used for library amplification, (ii) a 9 nt unique sequence specific to the antibody ID and (iii) a 8 nt Unique Molecular Identifier (UMI) for determining tag counts.

Bead Biotinylation

1 mL of Protein G Dynabeads (ThermoFisher, #10003D) were washed once with 1×PBSt (1×PBS+0.1% Tween-20) and resuspended in 1 mL PBSt. Beads were then incubated with μL of 5 mM EZ-Link Sulfo-NHS-Biotin (Thermo, #21217) on a HulaMixer for 30 minutes at room temperature. To quench the NHS reaction, beads were placed on a magnet, 500 μL of buffer was removed and replaced with 500 μL of 1M Tris pH 7.4 and beads were incubated on the HulaMixer for an additional 30 minutes at room temperature. Beads were then washed twice with 1 mL PBSt and resuspended in their original storage buffer until use.

Labeling Biotinylated Beads with Oligonucleotide Tags

Unique biotinylated oligonucleotides were first coupled to streptavidin (BioLegend, #280302) in a 96-well PCR plate. In each well, 20 μL of 10 μM oligo was added to 75 μL 1×PBS and 5 μL 1 mg/mL streptavidin. The 96-well plate was then incubated with shaking at 1600 rpm on a ThermoMixer for 30 minutes at room temperature. Each well was then diluted 1:4 in 1×PBS for a final concentration of 227 nM. This is the working stock.

For each desired target antibody in an experiment, 10 uL of biotinylated Protein G beads were prepared. All beads required for a single experiment were first pooled into a tube, washed in 1 mL of PBSt and then resuspended in 200 uL of 1× oligo binding buffer (0.5×PBST, 5 mM Tris pH 8.0, 0.5 mM EDTA, 1M NaCl) per 10 uL of beads. 200 μL of the bead suspension was aliquoted into individual wells of a deep well 96-well plate (Nunc 96-Well DeepWell Plates with Shared-Wall Technology, Thermo Scientific, Cat. No. 260251) followed by addition of 14 μL of 5.675 nM (1:40 from 227 nM working stock made fresh) streptavidin-coupled oligo to each well. The 96-well plate was then shaken at 1200 rpm on a ThermoMixer for 30 minutes at room temperature. Beads were then washed twice with M2 buffer (20 mM Tris 7.5, 50 mM NaCl, 0.2% Triton X-100, 0.2% Na-Deoxycholate, 0.2% NP-40), twice with 1×PBSt, and resuspended in 200 μL of 1×PBSt.

Binding Antibody to Labeled Protein G Beads

2.5 μg of each antibody was added to each well of the 96-well plate containing oligo labeled beads resuspended in 1×PBSt. The plate was incubated on a ThermoMixer overnight at 4 C with 30 seconds of shaking at 1200 RPM every 15 minutes. Beads were washed twice with 1×PBSt+2 mM biotin (Sigma, #B4639-5G), resuspended in 200 μL of 1×PBSt+2 mM biotin, and left shaking at 1200 rpm for 10 minutes at room temperature. All wells containing beads were then pooled together and washed twice with 1 mL 1×PBSt+2 mM biotin. This is the pool of beads with each bead having an Antibody ID oligo and a matched antibody.

Pooled Immunoprecipitation

For each experiment, fragmented lysate from 50,000 to 45,000,000 cells were prepared as described above. This was diluted with PBSt+10 mM biotin+1×PIC (final concentration). The pool of labeled beads was then added to the lysate and rotated on the hulu mixer for 1 hour at room temperature. Beads were then washed 2× with 1 mL IP Wash Buffer I (20 mM Tris pH 8.0, 0.05% SDS, 1% Triton X 100, 2 mM EDTA, 150 mM NaCl in water), II (20 mM Tris pH 8.0, 0.05% SDS, 1% Triton X 100, 2 mM EDTA, 500 mM NaCl in water) and SPRITE wash buffer (20 mM Tris pH7.5, 0.2% Triton X100, 0.2% NP-40, 0.2% DOC, and 50 mM NaCl).

Chromatin End Repair and dA-Tailing

To blunt end and phosphorylate double stranded DNA, the NEB End Repair Module (E6050L) was employed. Enzyme and buffer mixes from the kit were added to the beads and this mixture was incubated at 20 C for 15 minutes. The reaction was quenched with 3× volume of PBSt+50 uM EDTA, and then the beads were washed 2× with 1 mL PBSt. Next, DNA was dA-tailed using the NEBNext dA-tailing Module (E6053L). Enzyme and buffer mixes from the kit were added to the beads and this mixture was incubated at 37 C for 15 minutes. The reaction was quenched with 3× volume of PBSt+50 uM EDTA, and then the beads were washed 2× with 1 mL PBSt.

Split-and-Pool Barcoding to Identify DNA-Protein Interactions

Split-and-pool barcoding was performed as previously described (Quinodoz et al 2021) with minor modifications. Specifically, the number of barcoding rounds and number of tags used for each round was determined based on the number of beads that needed to be resolved. These parameters were selected to ensure that virtually all barcode clusters (>95%) represented molecules belonging to unique, individual beads. In most cases, 6 rounds of barcoding with 24-36 tags per round were performed. Each individual tag sequence was used in only a single round of barcoding. All split-and-pool ligation steps were performed for 5 minutes at room temperature and supplemented with 2 mM biotin and 5.4 uM ProteinG.

SPIDR

UV-Crosslinking for SPIDR

K562 cells (ATCC, CCL-243) and HEK293T cells (ATCC, CRL-3216) were purchased from ATCC and cultured under standard conditions. Crosslinking was performed as in Van Nostrand et al. 2016. Briefly, K562 cells were washed once with 1×PBS and diluted to a density of ˜10 million cells/mL in 1×PBS for plating onto culture dishes. HEK293T cells were washed once with 1×PBS and crosslinked directly on culture dishes. RNA-protein interactions were crosslinked on ice using 0.25 J cm⁻² (UV 2.5k) of UV at 254 nm in a Spectrolinker UV Crosslinker. Cells were then scraped from culture dishes, washed once with 1×PBS, pelleted by centrifugation at 330×g for 3 minutes, and flash-frozen in liquid nitrogen for storage at −80° C.

Bead Biotinylation/Labeling Biotinylated Beads with Oligonucleotide Tags/Binding Antibody to Labeled Protein G Beads

These procedures were performed as described above.

Pooled Immunoprecipitation (SPIDR)

For each experiment, 10 million cells were lysed in 1 mL RIPA buffer (50 mM HEPES pH 7.4, 100 mM NaCl, 1% NP-40, 0.5% Na-Deoxycholate, 0.1% SDS) supplemented with μL Protease Inhibitor Cocktail (Sigma, #P8340-5 mL), 10 μL of Turbo DNase (Invitrogen, #AM2238), 1× Manganese/Calcium mix (2.5 mM MnCl₂, 0.5 mM CaCl₂)), and 5 μL of RiboLock RNase Inhibitor (Thermo Fisher, #E00382)). Samples were incubated on ice for 10 minutes to allow lysis to proceed. After lysis, cells were sonicated at 3-4 W of power for 3 minutes (pulses 0.7 s on, 3.3 s off) using the Branson sonicator and then incubated at 37° C. for 10 minutes to allow for DNase digestion. DNase reaction was quenched with addition of 0.25 M EDTA/EGTA mix for a final concentration of 10 mM EDTA/EGTA. RNase If (NEB, #M0243L) was then added at a 1:500 dilution and samples were incubated at 37° C. for 10 minutes to allow partial fragmentation of RNA to obtain RNA of approximately ˜300-400 bp in length. RNase reaction was quenched with addition of 500 μL ice old RIPA buffer supplemented with 20 μL Protease Inhibitor Cocktail and 5 μL of RiboLock RNase Inhibitor, followed by incubation on ice for 3 minutes. Lysates were then cleared by centrifugation at 15000×g at 4° C. for 2 minutes. The supernatant was transferred to new tubes and diluted in additional RIPA buffer such that the final volume corresponded to 1 mL lysate for every 100 μL of Protein G beads used. Lysate was then combined with the labeled antibody-bead pool and 1 M biotin was added to a final concentration of 10 mM as to quench any disassociated streptavidin-coupled oligos. Beads were left rotating overnight at 4° C. on a HulaMixer. Following immunoprecipitation, beads were washed twice with RIPA buffer, twice with high salt wash buffer (50 mM HEPES pH 7.4, 1 M NaCl, 1% NP-40, 0.5% Na-Deoxycholate, 0.1% SDS), and twice with Tween buffer (50 mM HEPES pH 7.4, 0.1% Tween-20).

Ligation of the RNA Phosphate Modified (“RPM”) Tag

After immunoprecipitation, 3′ ends of RNA were modified to have 3′ OH groups compatible for ligation using T4 Polynucleotide Kinase (NEB, #M0201L). Beads were incubated at 37° C. for 10 minutes with shaking at 1200 rpm on a ThermoMixer. Following end repair, beads were buffer exchanged by washing twice with high salt wash buffer and twice with Tween buffer. RNA is subsequently ligated with an “RNA Phosphate Modified” (RPM) adaptor (See, e.g., Quinodoz et al 2021) using High ConcentrationT4 RNA Ligase I (NEB, M0437M). Beads were incubated at 24° C. for 1 hour 15 minutes with shaking at 1400 rpm, followed by three washes in Tween buffer. After RPM ligation, RNA was converted to cDNA using SuperScript III (Invitrogen, #18080093) at 42° C. for 20 minutes using the “RPM Bottom” RT primer to facilitate on-bead library construction and a 5′ sticky end to ligate tags during split-and-pool barcoding. Excess primer is digested with Exonuclease I (NEB, #M0293L) at 37° C. for 15 minutes.

Split-and-Pool Barcoding to Identify RNA Protein Interactions

Split-and-pool barcoding was performed as previously described (Quinodoz et al 2021) with minor modifications. Specifically, beads were split-and-pool ligated over ≥6 rounds with a set of “Odd,” “Even,” and “Terminal” tags. The number of barcoding rounds performed for each SPIDR experiment was determined based on the complexity of the given bead pool. It was ensured that virtually all barcode clusters (>95%) represented molecules belonging to unique, individual beads. All split-and-pool ligation steps were performed for 5 minutes at room temperature and supplemented with 2 mM biotin and 1:40 RiboLock RNase Inhibitor to prevent RNA degradation.

Chromatin End Repair and dA-Tailing

The DNA ends was then repaired to enable ligation of tags to each molecule.

Specifically, the 5′ ends of double-stranded DNA were blunt ended and phosphorylated using two enzymes. First, T4 Polynucleotide Kinase (NEB) treatment is performed at 37 C for 1 hr, the enzyme is quenched using 1 mL Modified RLT buffer, and then buffer is exchanged with two washes of 1 mL SPRITE Detergent Buffer to beads at room temperature. Next, the NEBNext End Repair Enzyme cocktail (containing T4 DNA Polymerase and T4 PNK) and 1×NEBNext End Repair Reaction Buffer is added to beads and incubated at 20 C for 1 hr, and inactivated and buffer exchanged as specified above. DNA was then dA-tailed using the Klenow fragment (50-30 exo-, NEBNext dA-tailing Module) at 37 C for 1 hr, and inactivated and buffer exchanged as specified above.

Split and pool barcoding was performed as described herein.

Library Preparation (SPIDR)

After split-and-pool barcoding, beads were aliquoted into 5% aliquots for library preparation and sequencing. RNA in each aliquot was degraded by incubating with RNase H (NEB, #M0297L) and RNase cocktail (Invitrogen, #AM2286) at 37° C. for 20 minutes. 3′ ends of the resulting cDNA were ligated to attach dsDNA oligos containing library amplification sequences using a “splint” ligation as previously described (Quinodoz et al 2021). The “splint” ligation reaction was performed with 1× Instant Sticky End Master Mix (NEB #M0370) at 24° C. for 1 hour with shaking at 1400 rpm on a ThermoMixer. Barcoded cDNA and biotinylated oligo tags were then eluted from beads by boiling in NLS elution buffer (20 mM Tris-HCl pH 7.5, 10 mM EDTA, 2% N-lauroylsarcosine, 2.5 mM TCEP) for 6 minutes at 91° C., with shaking at 1350 rpm.

Biotinylated oligo tags were first captured by diluting the eluant in 1× oligo binding buffer (0.5×PBST, 5 mM Tris pH 8.0, 0.5 mM EDTA, 1M NaCl) and subsequently binding to MyOne Streptavidin C1 Dynabeads (Invitrogen, #65001) at room temperature for 30 minutes. Beads were placed on a magnet and the supernatant, containing cDNA, was moved to a separate tube. Biotinylated oligo tags were amplified on-bead using 2×Q5 Hot-Start Mastermix (NEB #M0494) with primers that add the indexed full Illumina adaptor sequences.

To isolate barcoded cDNA, the supernatant was first incubated with a biotinylated antisense ssDNA (“anti-RPM”) probe that hybridizes to the junction between the reverse transcription primer and splint sequences to reduce empty insertion products. This mixture was then bound to MyOne Streptavidin C1 Dynabeads at room temperature for 30 minutes. Beads were placed on a magnet and the supernatant, containing the remaining cDNA products, was cleaned up on Silane beads (Invitrogen, #37002D) as previously described (Shishkin et al 2015). Finally, cDNA was amplified using 2×Q5 Hot-Start Mastermix (NEB #M0494) with primers that add the indexed full Illumina adaptor sequences.

After amplification, libraries were cleaned up using 1×SPRI (AMPure XP), size-selected on a 2% agarose gel, and cut at either ˜300 nt (barcoded oligo tag) or between 300-1000 nt (barcoded cDNA). Libraries were subsequently purified with Zymoclean Gel DNA Recovery Kit (Zymo Research, #4007).

Sequencing

Paired-end sequencing was performed on either an Illumina NovaSeq 6000 (S4 flowcell), NextSeq 550, or NextSeq 2000 with read length³ 100×200 nucleotides. For the K562 data, 37 SPIDR aliquots were generated and sequenced from two technical replicate experiments. The two experiments were generated using the same batch of UV-crosslinked lysate processed on the same day. Each SPIDR library corresponds to a distinct aliquot that was separately amplified with different indexed primers, providing an additional round of barcoding as previously described (Quinodoz et al 2021). Minimum required sequencing depth for each experiment was determined by the estimated number of beads and unique molecules in each aliquot. For oligo tag libraries, each library was sequenced to a depth of observing ˜5 unique oligo tags per bead on average. For cDNA libraries, each library was sequenced with at least 2× coverage of the total estimated library complexity.

Read Processing and Alignment (SPIDR)

Paired-end RNA sequencing reads were trimmed to remove adaptor sequences using Trim Galore! v0.6.2 and assessed with FastQC v0.11.8. Subsequently, the RPM (ATCAGCACTTA) sequence was trimmed using Cutadapt v3.4 from both 5′ and 3′ read ends. The barcodes of trimmed reads were identified with Barcode ID v1.2.0 (https://github.com/GuttmanLab/sprite2.0-pipeline) and the ligation efficiency was assessed. Reads with or without an RPM sequence were split into two separate files to process RNA and oligo tag reads individually downstream, respectively.

RNA read pairs were then aligned to a combined genome reference containing the sequences of repetitive and structural RNAs (ribosomal RNAs, snRNAs, snoRNAs, 45S pre-rRNAs, tRNAs) using Bowtie2. The remaining reads were then aligned to the human (hg38) genome using STAR aligner. Only reads that mapped uniquely to the genome were kept for further analysis.

Barcode Matching and Filtering (SPIDR)

Mapped RNA and oligo tag reads were merged, and a cluster file was generated for all downstream analysis as previously described. MultiQC v1.6 was used to aggregate all reports. To unambiguously exclude ligation events that could not have occurred sequentially, unique sets of barcodes were utilized for each round of split-and-pool. All clusters containing barcode strings that were out-of-order or contained identical repeats of barcodes were filtered from the merged cluster file. To determine the amount of unique oligo tags present in each cluster, sequences sharing the same Unique Molecular Identifier (UMI) were removed and the remaining occurrences were counted. To remove PCR duplication events within the RNA library, sequences sharing identical start and stop genomic positions were removed.

Splitting Alignment Files by Protein Identity

Barcode strings from filtered cluster files were then used to assign protein identities to the alignment file containing all mapped RNA reads. Because each cluster represents an individual bead, the frequency of oligo tags (each representing unique protein type) was used to determine protein assignments. Specifically, for each cluster ≥3 observed oligo tags was required and that the most common protein type represented ≥80% of all observed tags. RNA reads were then split into separate alignment files by barcode strings corresponding to protein type.

### Example 1

### ChIP-DIP

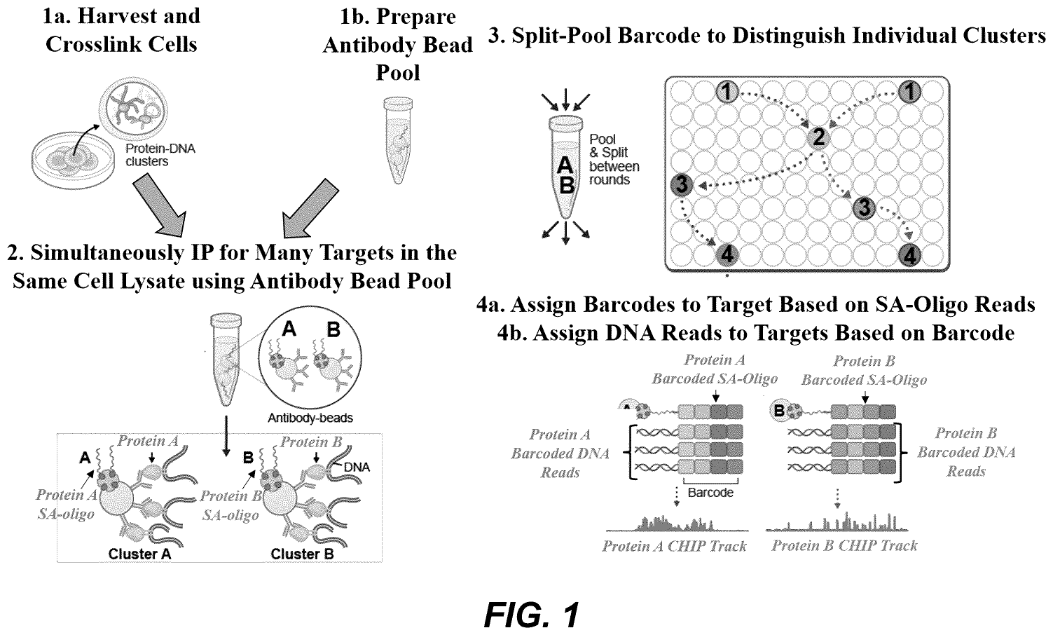

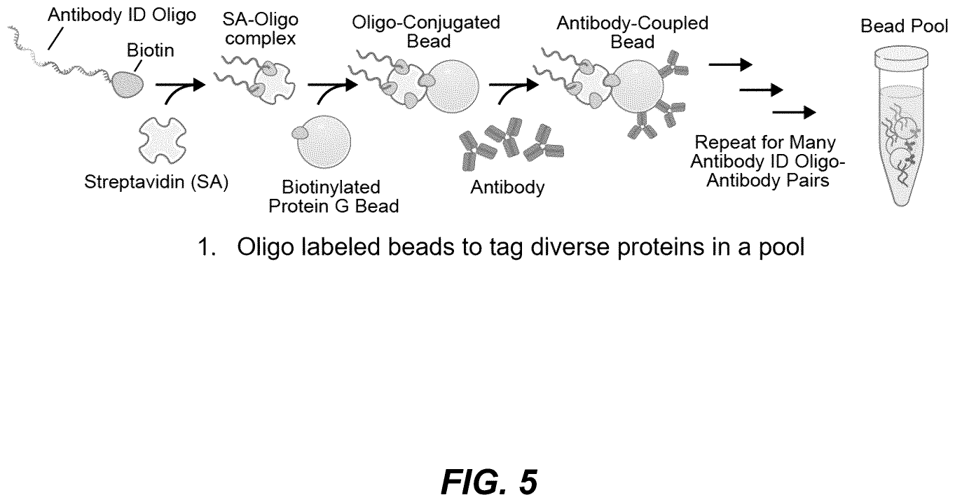

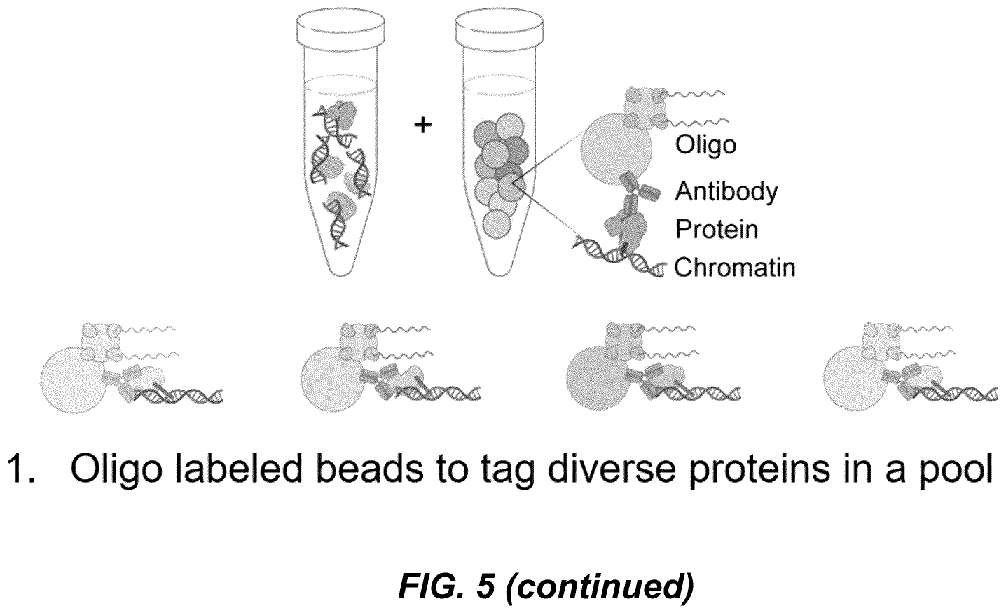

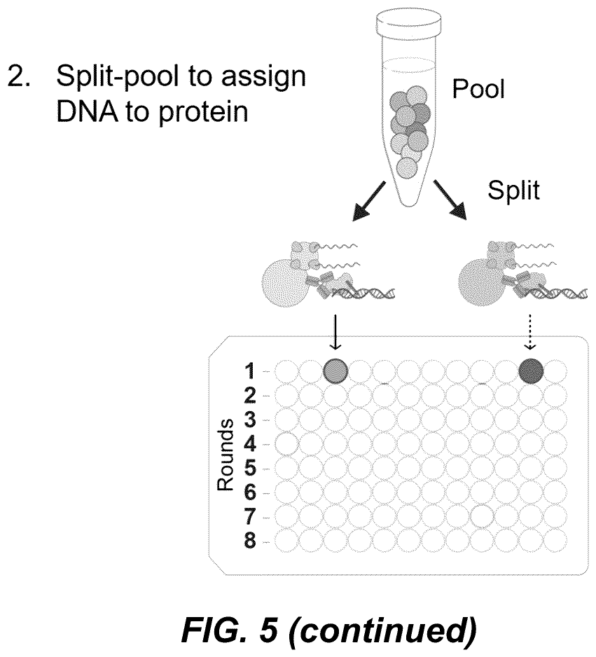

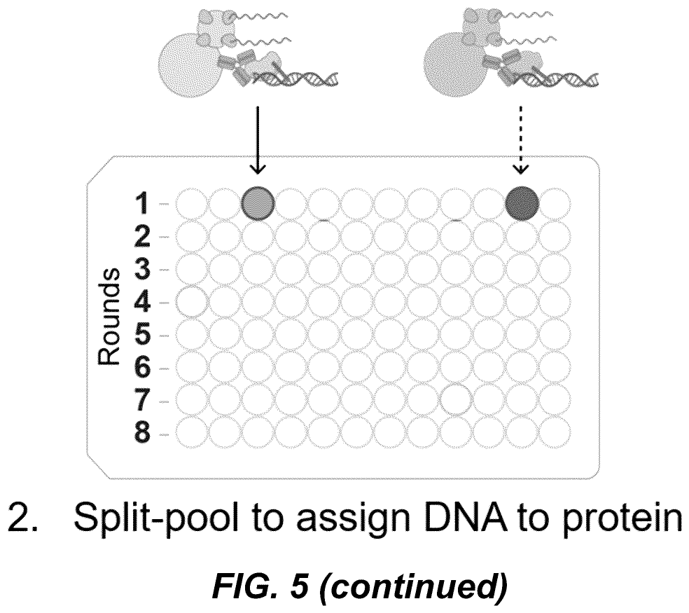

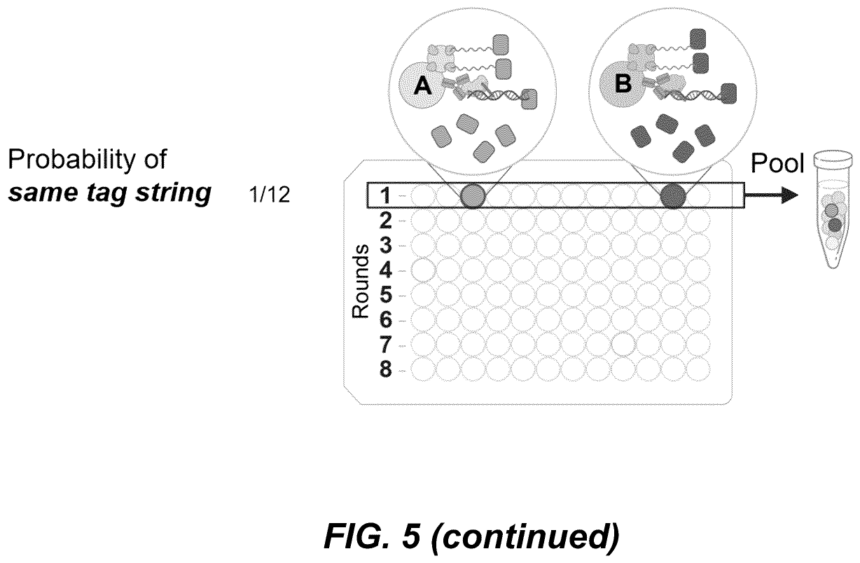

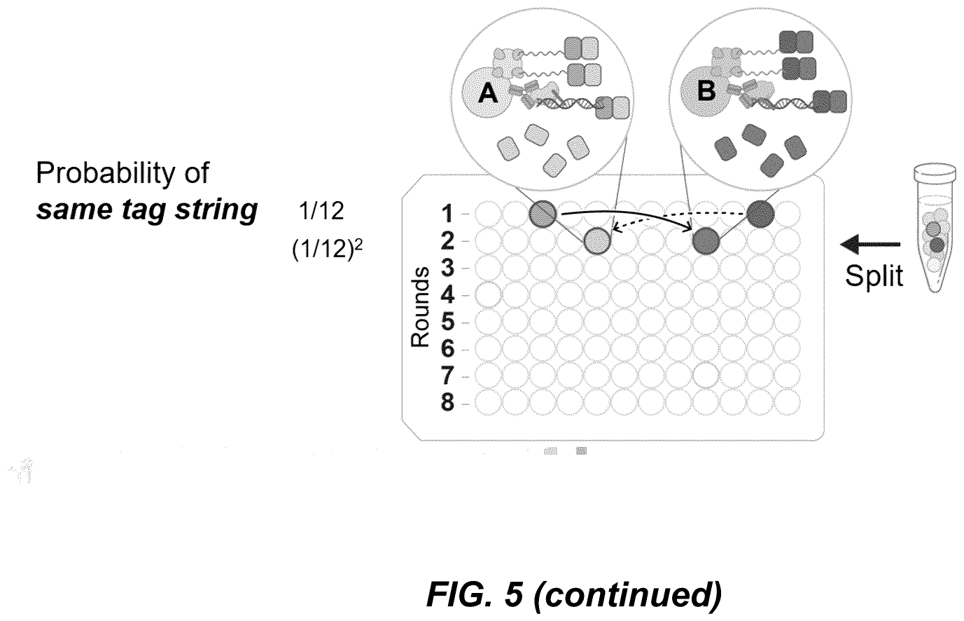

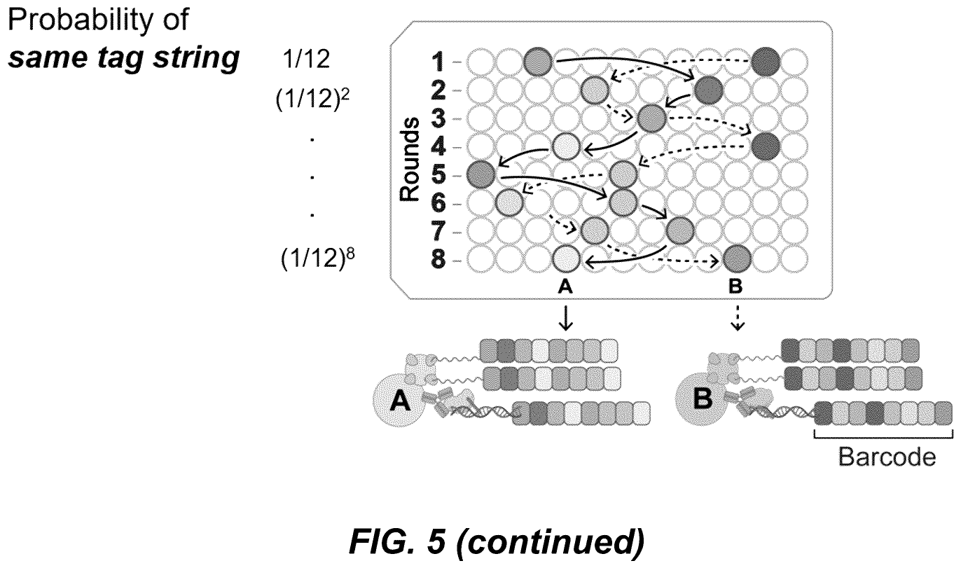

This example provides proof of concept for the ChIP-DIP compositions, methods, and systems provided herein. FIG. 4 depicts a non-limiting exemplary schematic related to the challenge of elucidating gene regulation via mapping of proteins on chromatin. FIG. 1 and FIG. 5 depict non-limiting exemplary schematics of the ChIP-DIP workflow. Each antibody can be associated with a unique “SA-Oligo” (aka antibody ID oligo) that is bound to the same Protein G bead and sequencing the SA-Oligo enables identification of the antibody on each, individual Protein G bead. Specifically, matching of split-pool barcodes allows SA-oligos bound to the same bead to be grouped together into a barcoded cluster. Barcoded clusters are highly enriched for a single type SA-Oligo, allowing for unambiguous assignment of the barcoded clusters to individual antibodies. To enable this one-to-one pairing, the number of possible split-pool barcodes is significantly higher than the total number of beads, such that the probability that any two beads share the same split-pool barcode is very low.

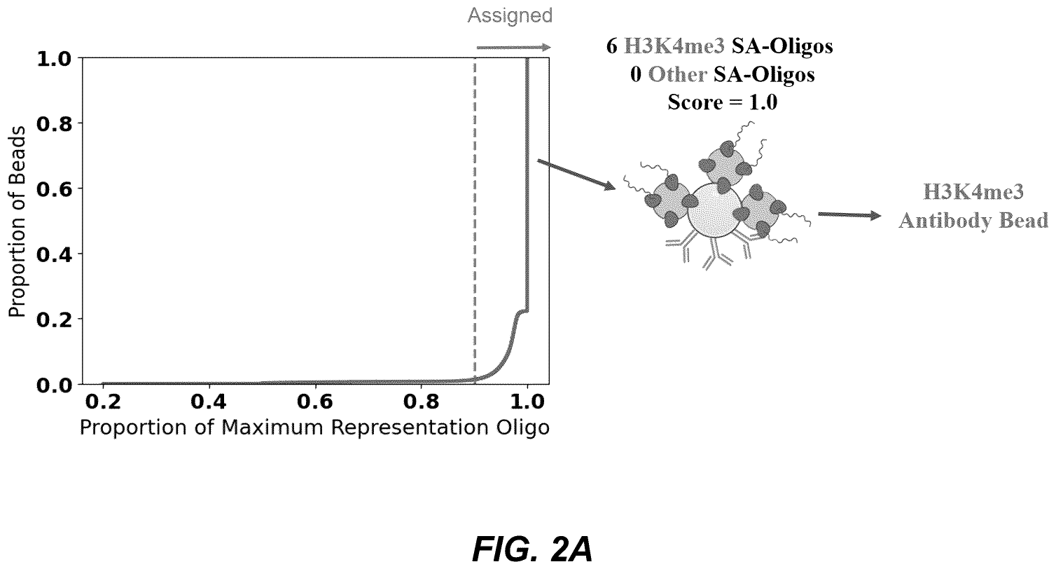

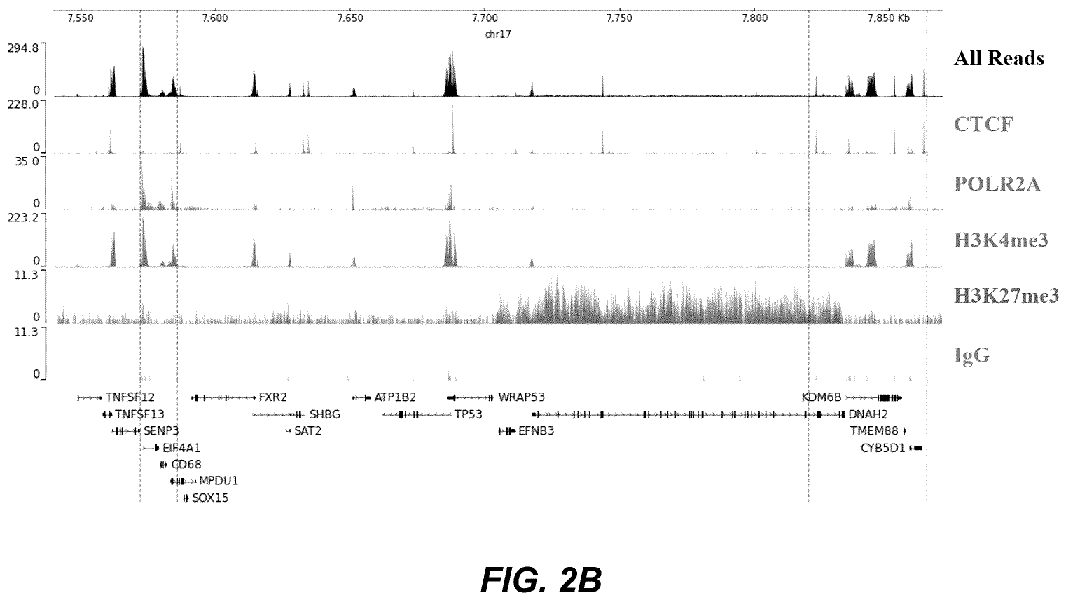

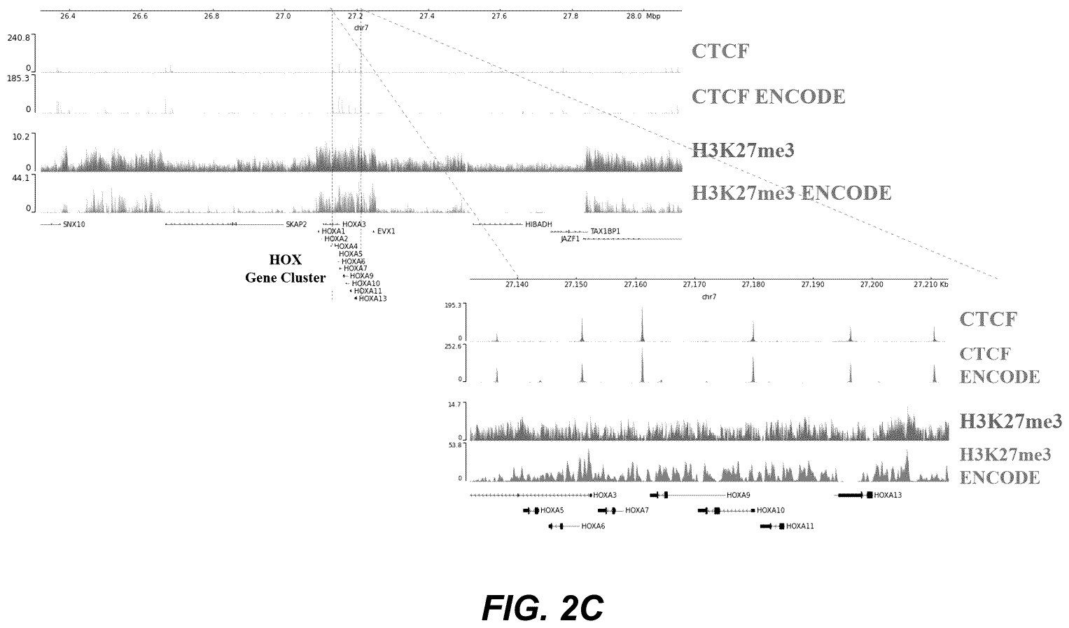

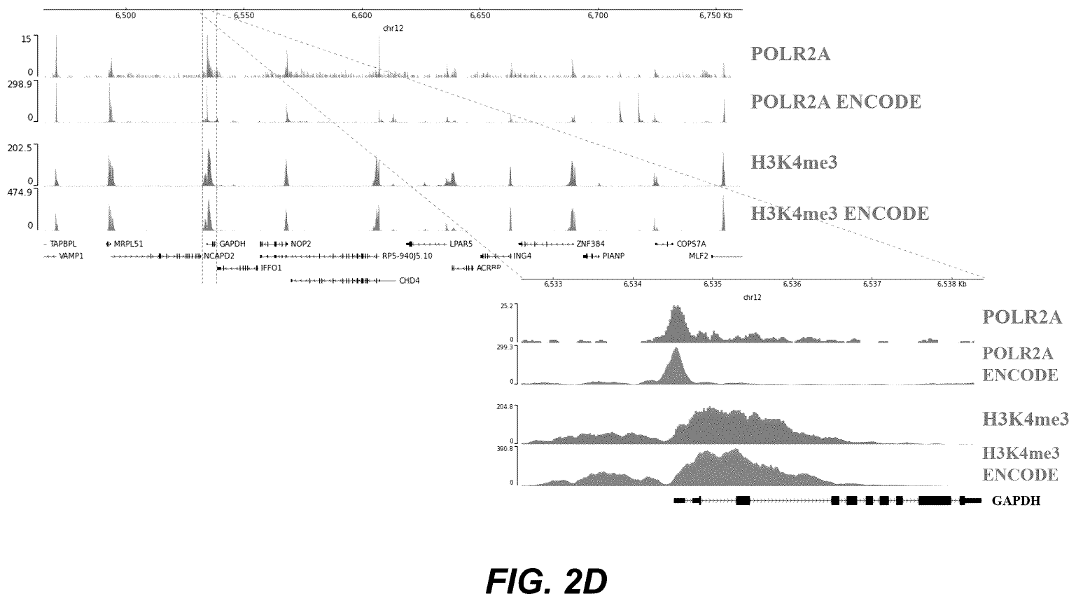

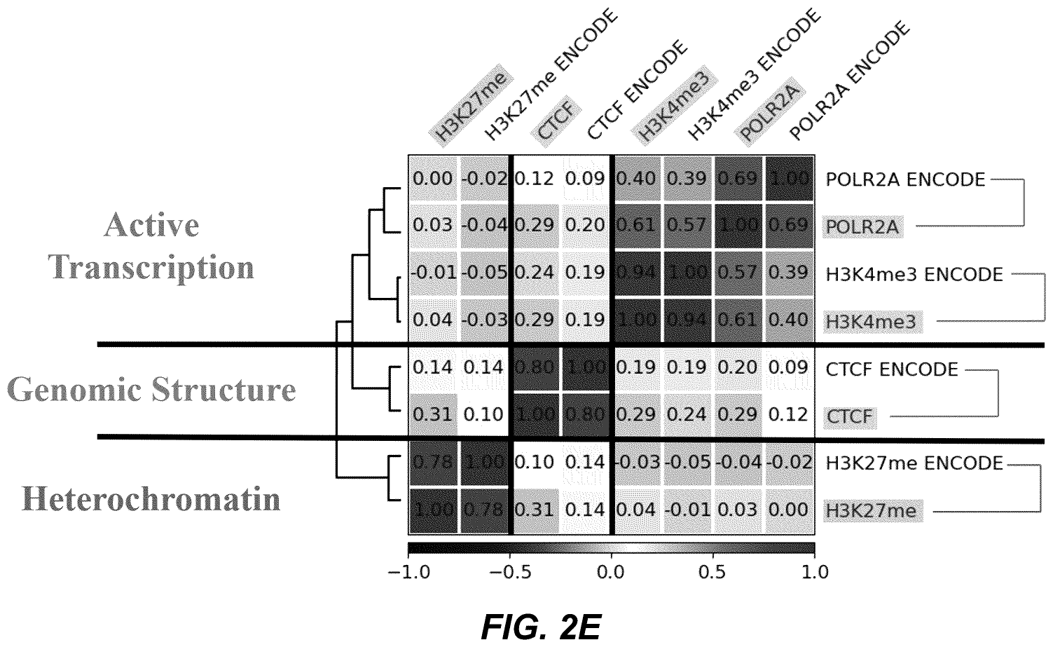

FIGS. 2A-2E depict data related to small proof-of-concept panel (CTCF, POLR2A, H3K4me3, H3K27me3, and IgG) ChIP-DIP experiment, including the assigning of cluster barcodes to target proteins using SA-Oligo reads (FIG. 2A), separated tracks of individual target proteins (FIG. 2B), track comparison with ENCODE (FIG. 2C and FIG. 2D), and track correlations with ENCODE (FIG. 2E). With regards to the experiment depicted in FIG. 2A, unique biotinylated oligonucleotides were first coupled to streptavidin (BioLegend, #280302) in a 96-well PCR plate. In each well, 20 μL of 10 μM oligo was added to 75 μL 1×PBS and 5 μL 1 mg/mL streptavidin. The 96-well plate was then incubated with shaking at 1600 rpm on a ThermoMixer for 30 minutes at room temperature. Each well was then diluted 1:4 in 1×PBS for a final concentration of 227 nM (this is the working stock). For each antibody used in the experiment, 10 uL of biotinylated Protein G beads were then prepared. First, all biotinylated Protein G beads to be used in the experiment were pooled, washed in 1 mL of PBSt and then resuspended in 200 uL of 1× oligo binding buffer (0.5×PBST, 5 mM Tris pH 8.0, 0.5 mM EDTA, 1M NaCl) per 10 uL of beds. 200 μL of the bead suspension was aliquoted into individual wells of a deep well 96-well plate (Nunc 96-Well DeepWell Plates with Shared-Wall Technology, Thermo Scientific, Cat. No. 260251). To couple biotinylated Protein G beads with SA-oligos, 14 μL of 5.675 nM (1:40 from 227 nM working stock made fresh) streptavidin-coupled oligo was added to each well. The 96-well plate was then shaken at 1200 rpm on a ThermoMixer for 30 minutes at room temperature. Beads were then washed twice with M2 buffer (20 mM Tris 7.5, 50 mM NaCl, 0.2% Triton X-100, 0.2% Na-Deoxycholate, 0.2% NP-40), twice with 1×PBSt, and resuspended in 200 μL of 1×PBSt. Using the protocol outlined above, beads from various antibodies were pooled together, immunoprecipitation was performed with a fragmented chromatin sample and then split pool barcoding was performed. Molecules with the same string of split pool tags (Quinodoz et al, 2018) were grouped together into barcoded clusters. Then, the frequency of each antibody ID oligo in each cluster was calculated. For example, if there were 5 oligos for antibody A and 1 oligo for antibody B in a given cluster, oligo A had a frequency of 83.3% and is the oligo with the max representation. Calculation of the maximum representation frequency was repeated for each cluster and plotted as an ECDF in FIG. 2A. With regards to FIGS. 2B-2D, 5 sets of beads were coupled with 5 unique bead oligos as outlined in FIG. 5 and then a different antibody was coupled to each uniquely labeled set of beads. Beads were pooled together, an IP using K562 fragmented chromatin was performed, split pool was performed, and the resulting libraries were sequenced on a NextSeq 2000. Assignment of DNA reads to each antibody was performed by grouping molecules with the same string of split pool tags, as described above. All DNA reads corresponding to the same antibody were combined and aligned to the hg38 genome. The resulting alignments were visualized alongside reference data downloaded from the ENCODE portal on the Integrated Genome Viewer (IGV) and screenshots were used to generate the figures.

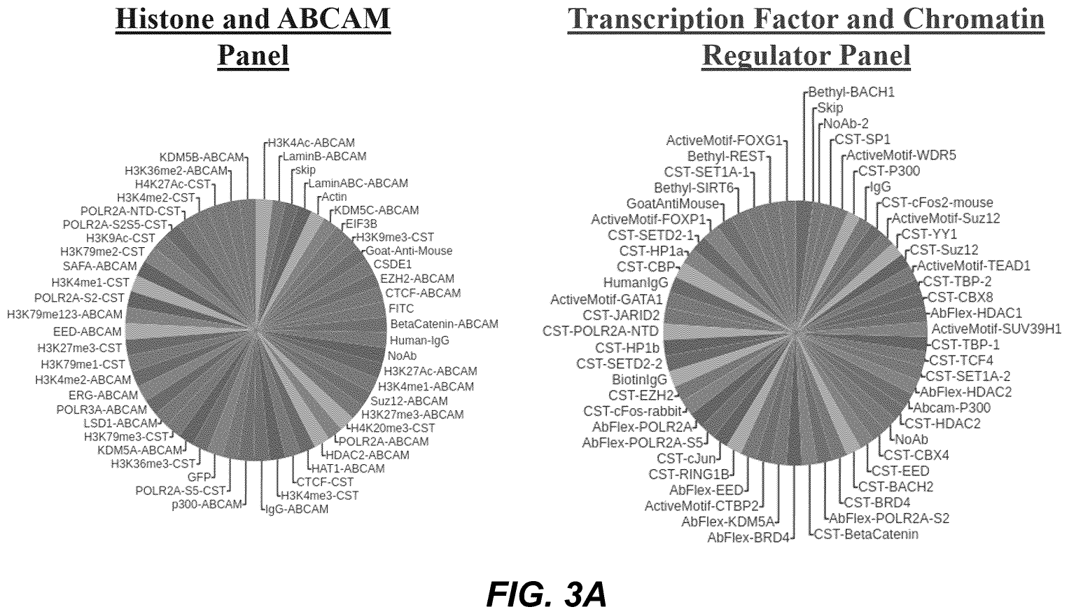

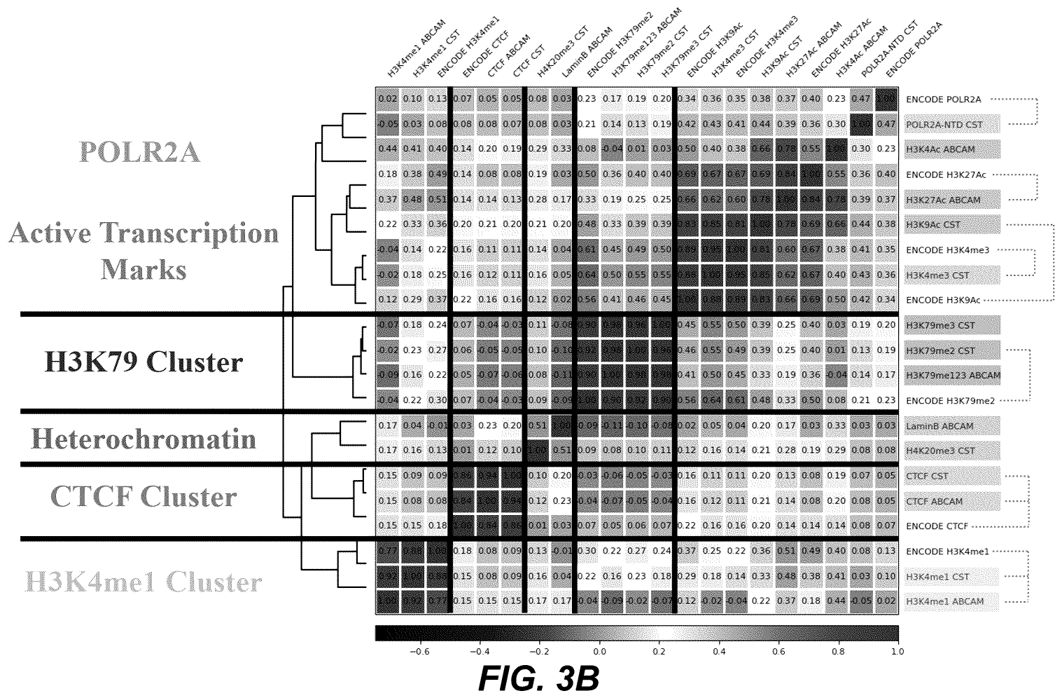

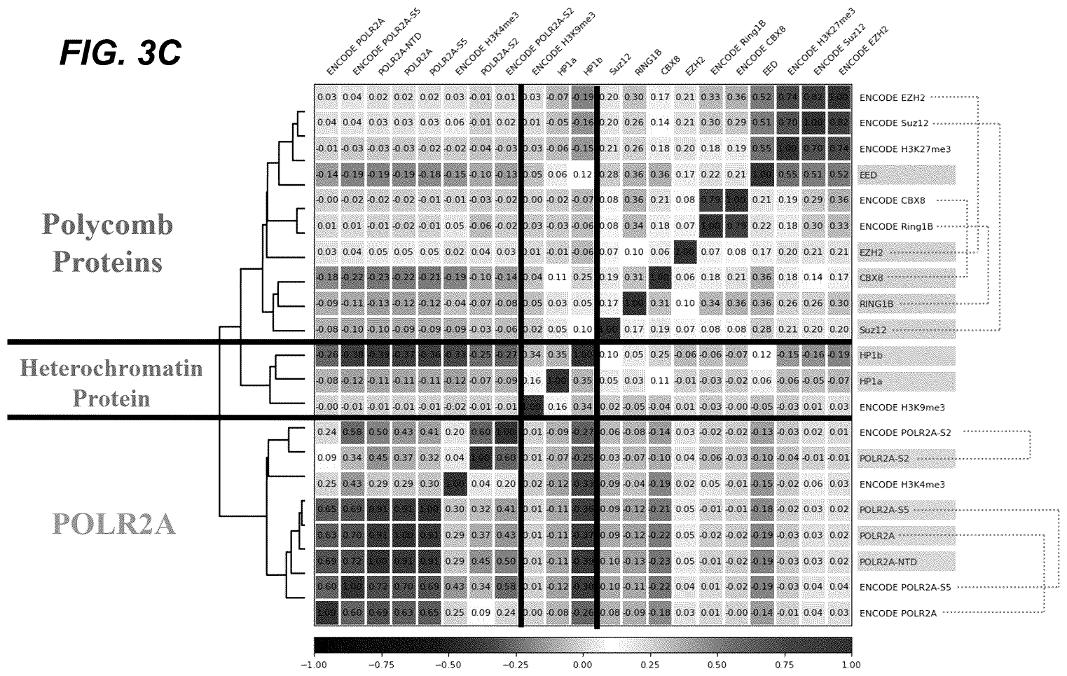

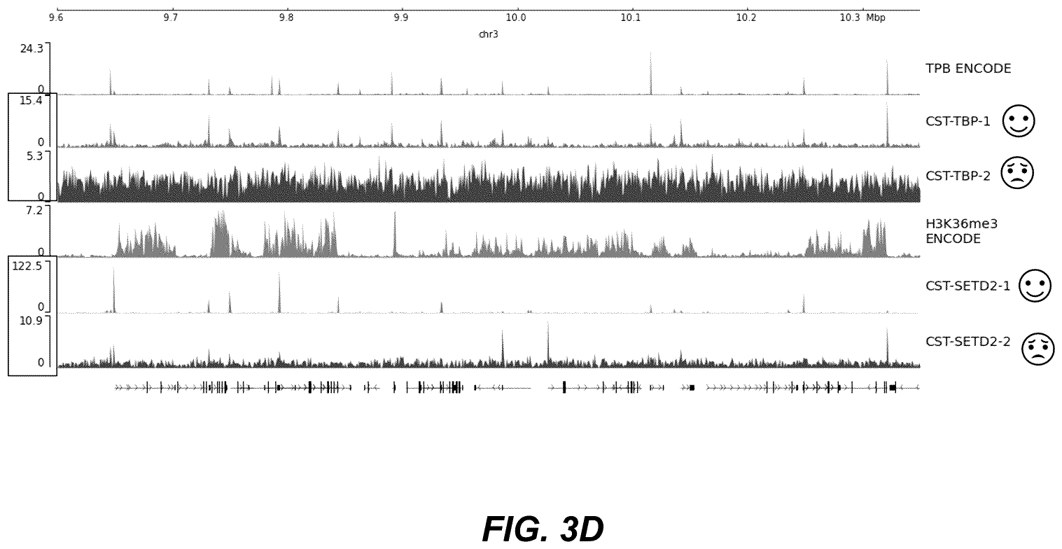

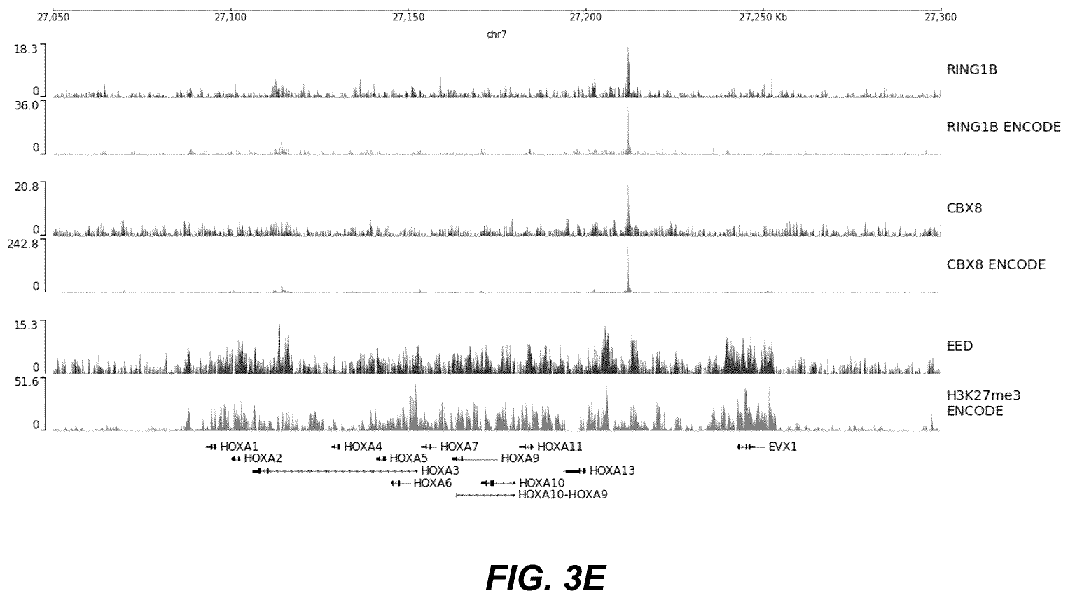

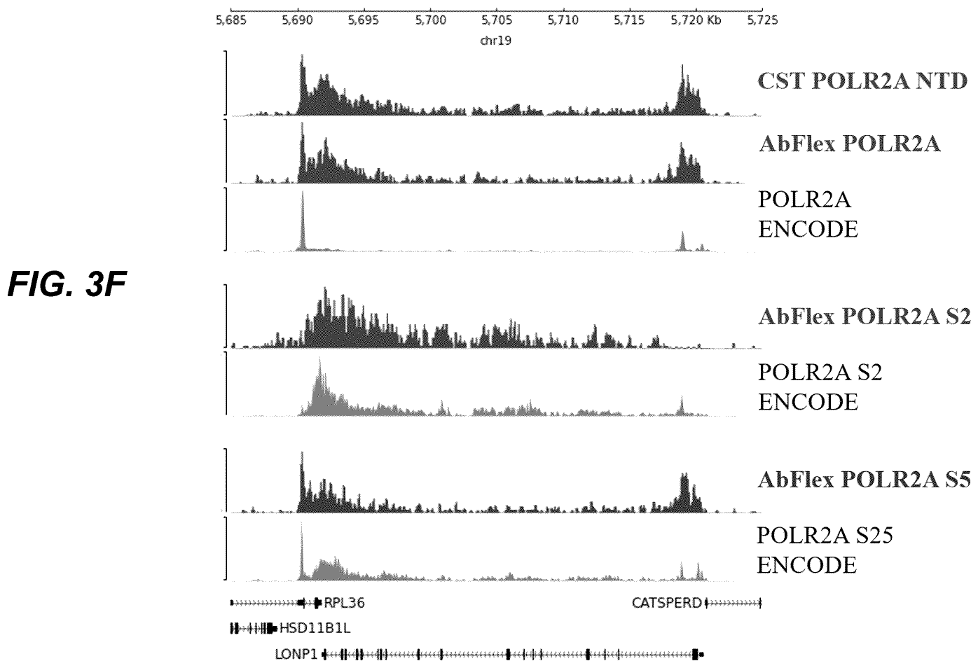

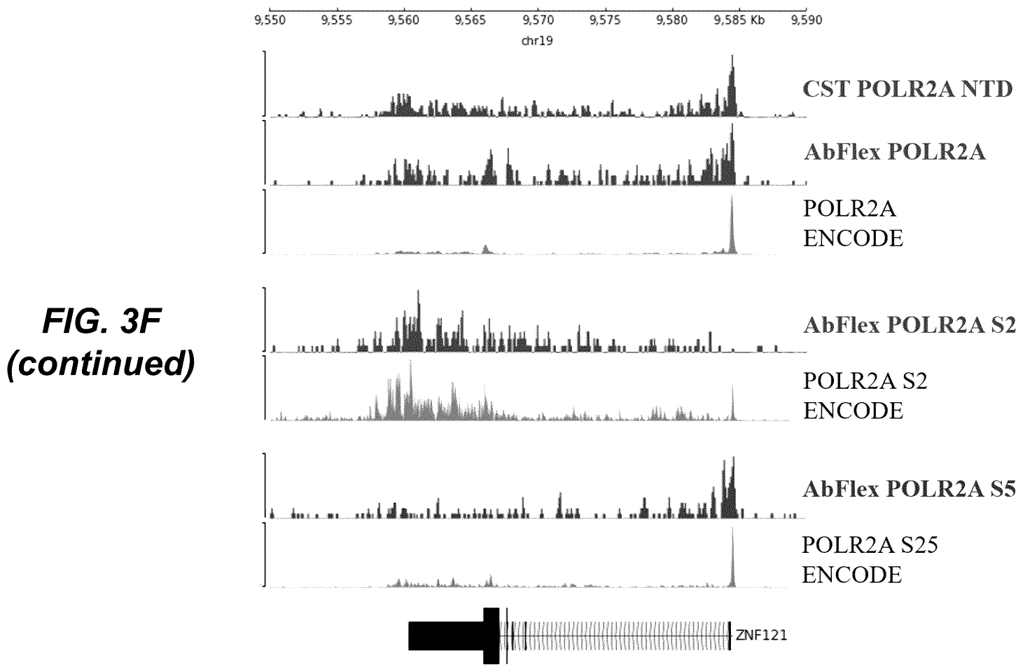

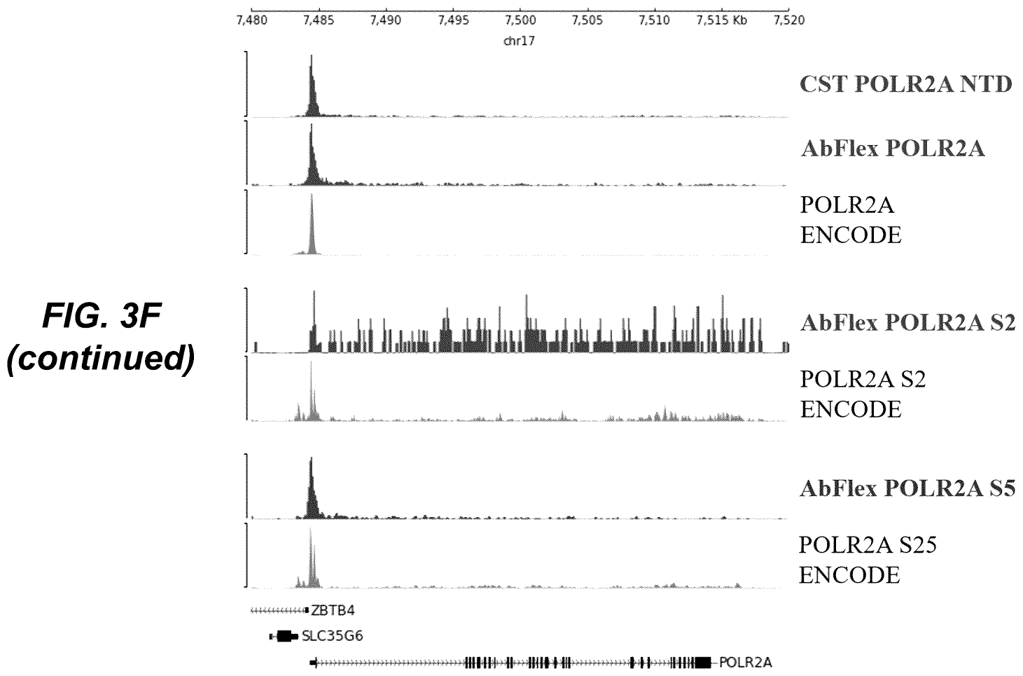

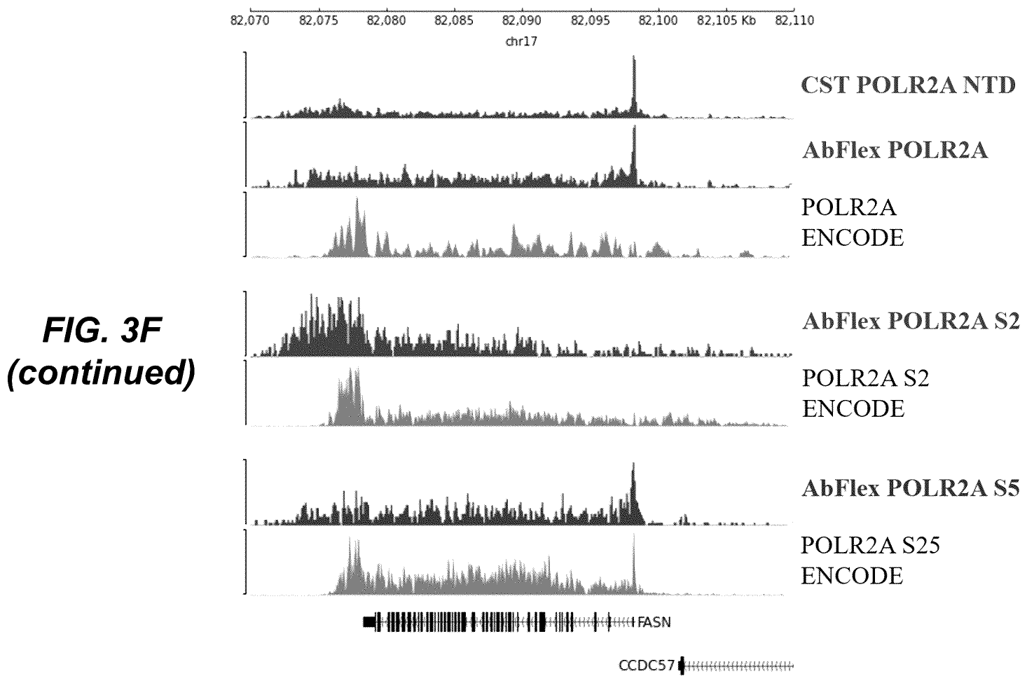

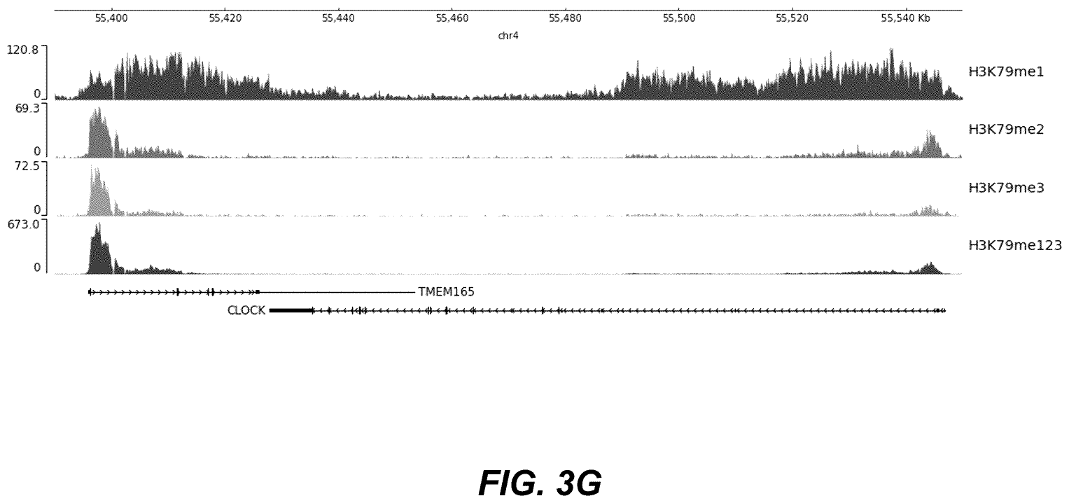

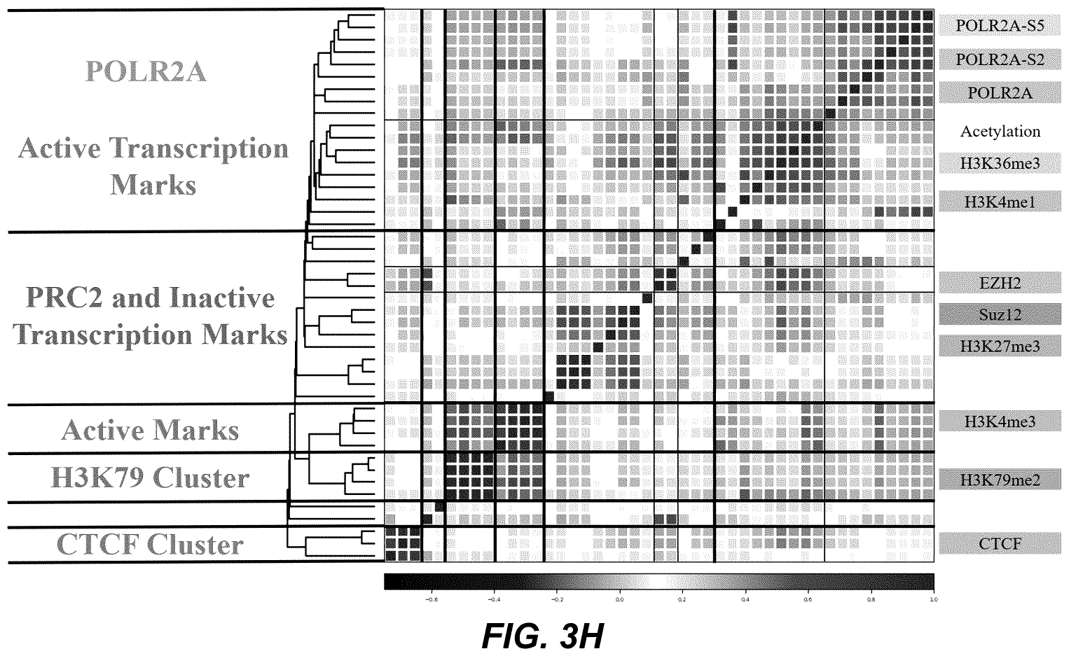

A set of larger proof-of-concept ChIP-DIP experiments was performed, interrogating over 50 diverse targets (a Histone and ABCAM Panel, a Transcription Factor and Chromatin Regulator Panel and an All Class of Targets Panel), FIGS. 3A-3H, depict the results of these experiments, including the distribution of beads assigned to each target (Histone and ABCAM Panel and Transcription Factor and Chromatin Regulator Panel; FIG. 3A), Pearson correlation matrices comparing track coverage with ENCODE (Histone and ABCAM Panel, FIG. 3B; Transcription Factor and Chromatin Regulator Panel, FIG. 3C), a comparison of the performance of multiple antibodies targeting the same protein (Transcription Factor and Chromatin Regulator Panel, FIG. 3D), visualization of multiple targets responsible for silencing at the Hox Gene Cluster (Transcription Factor and Chromatin Regulator Panel, FIG. 3E), visualization of various phosphorylation states of POLR2A (Transcription Factor and Chromatin Regulator Panel, FIG. 3F), visualization of various methylation states of H3K79. (Histone and ABCAM Panel, FIG. 3G), and a Pearson correlation matrix comparing track coverage with ENCODE over a diverse set of targets (All Class of Targets Panel, FIG. 311). With regards to FIGS. 3C-3G, the data was generated the same way as in FIGS. 2B-2D, but the pools of antibody-bead-oligo were from two different experiments using 55 and 66 different antibodies. The data was then visualized on IGV. A Pearson correlation matrix was generated in FIG. 2E, FIG. 3B, FIG. 3C, and FIG. 311 using DeepTools (https://deeptools.readthedocs.io/en/develop/content/list_of_tools.html).

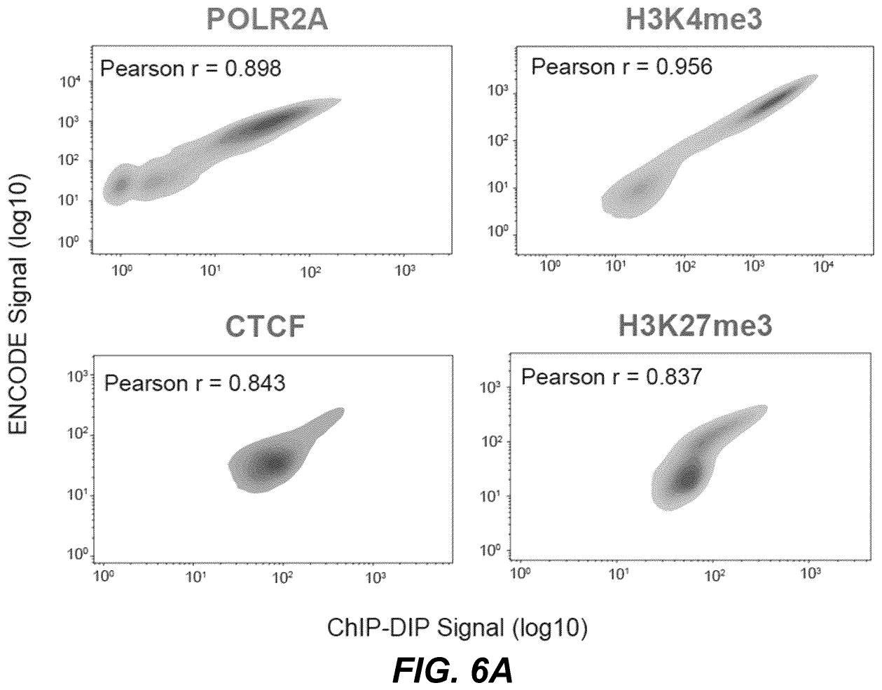

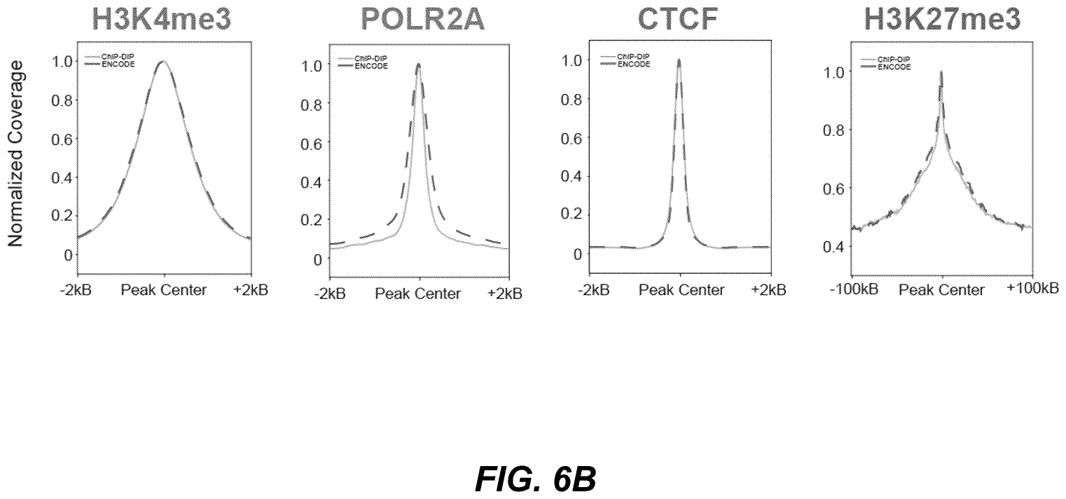

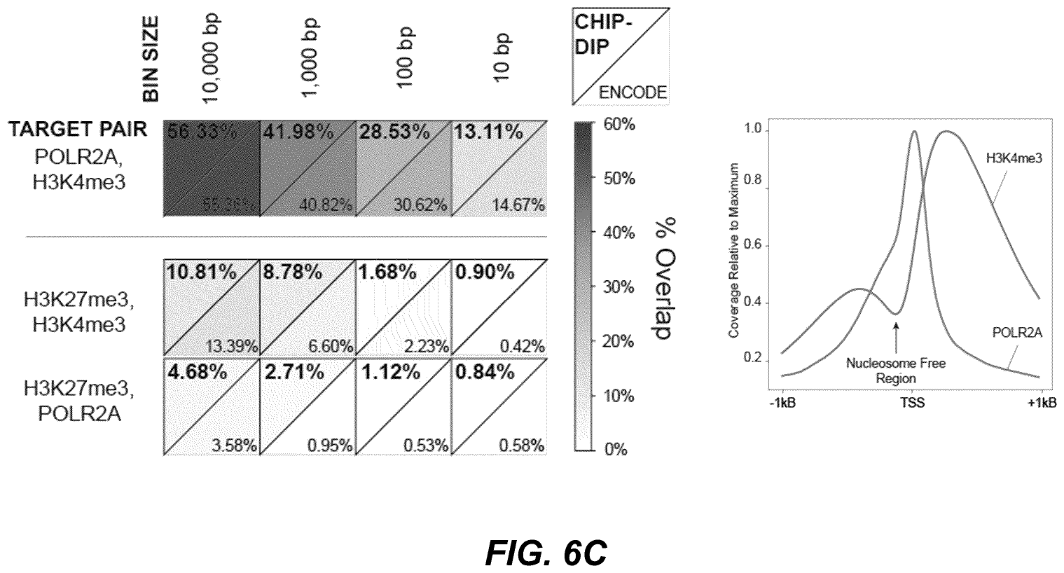

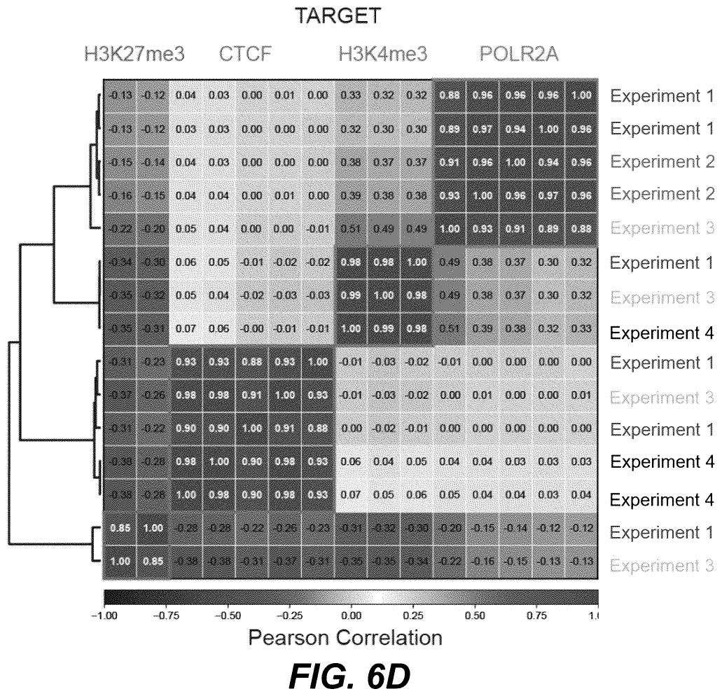

FIGS. 6A-6D depict data related to ChIP-DIP experiments interrogating a panel of model proteins (CTCF (chromatin loop formation), POLR2A (active transcription), H3K4me3 (active promoters), H3K27me3 (transcriptionally silenced chromatin), and IgG (negative control)), including signal (log 10) comparisons with ENCODE (FIG. 6A), peak-centered coverage comparison with ENCODE (FIG. 6B), sensitivity and specificity of target detection relative to ENCODE (FIG. 6C), and ChIP-DIP reproducibility (FIG. 6D). The experiment shown in FIGS. 6A-C is identical to that shown in FIGS. 2B-2E. FIG. 6A shows genome-wide Pearson correlations between ENCODE and ChIP DIP generated data. FIG. 6B shows a comparison of ChIP DIP data and ENCODE data at peaks called using Homer's “Call Peaks” program on ChIP DIP generated data. FIG. 6D shows Pearson correlations between multiple ChIP-DIP experiments at peak sites called on ChIP-DIP generated data.

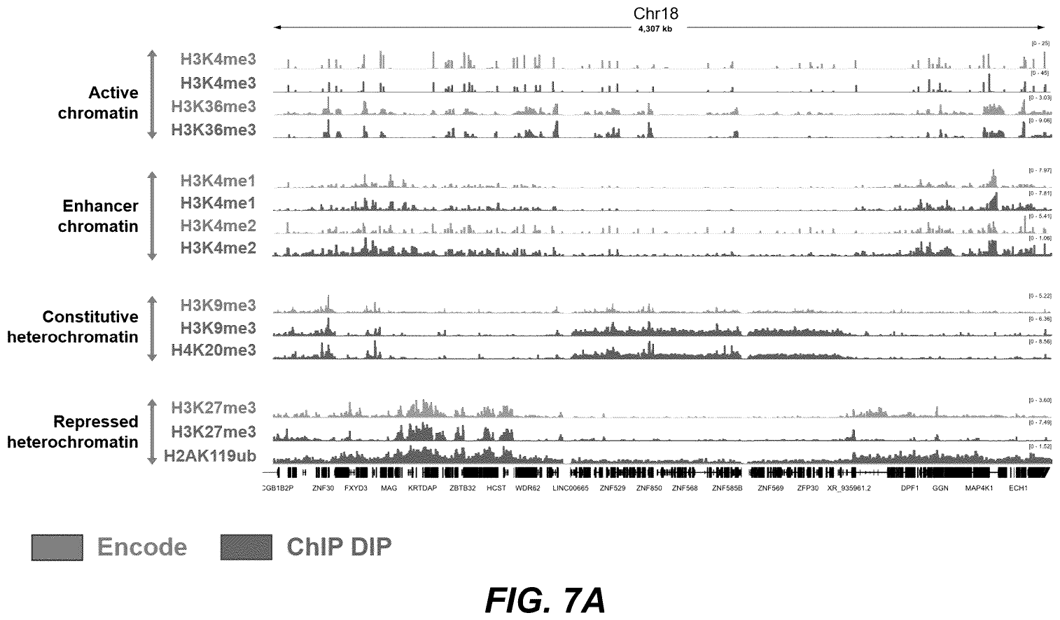

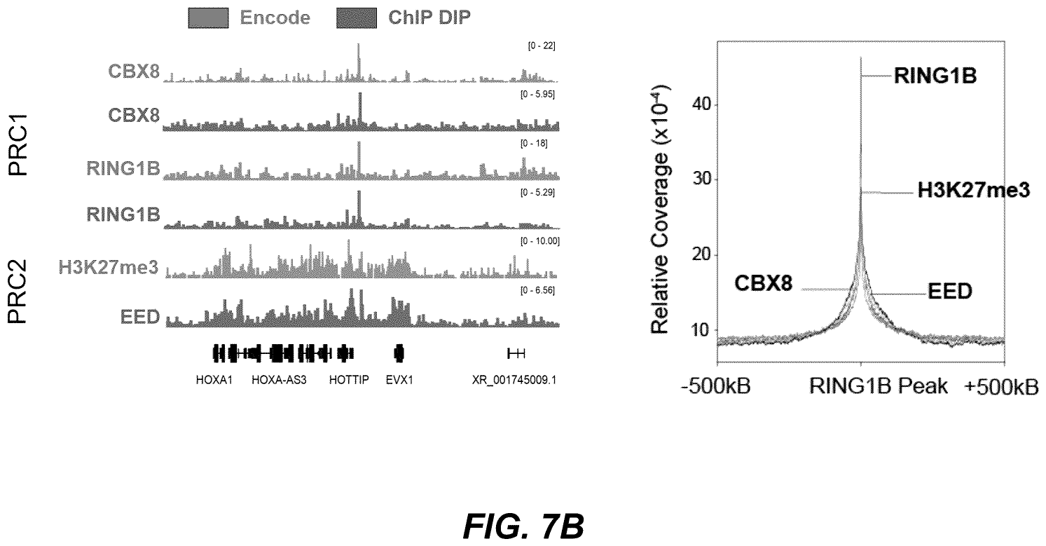

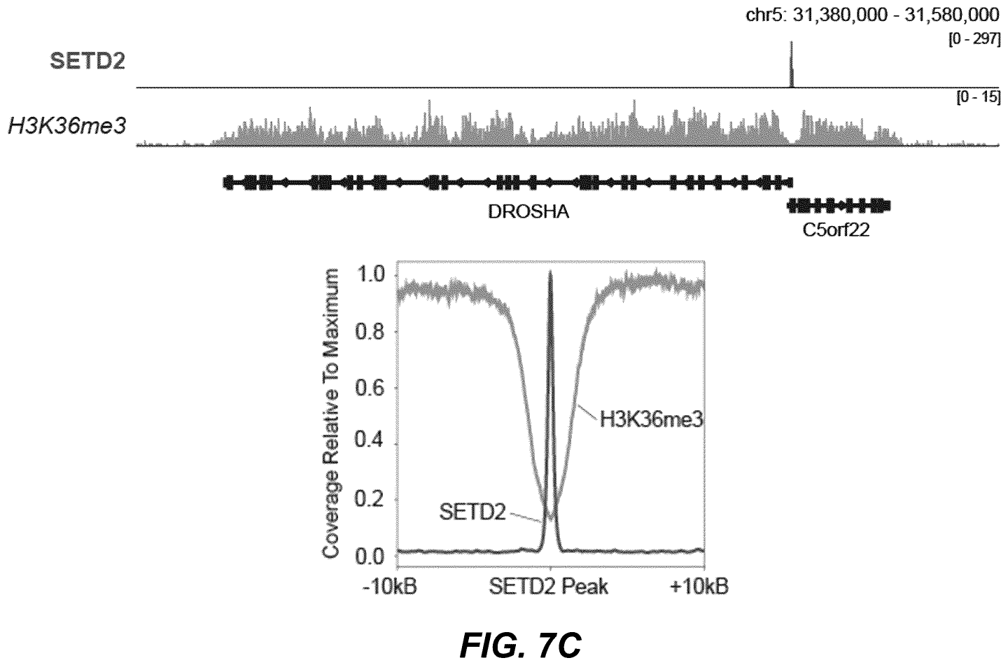

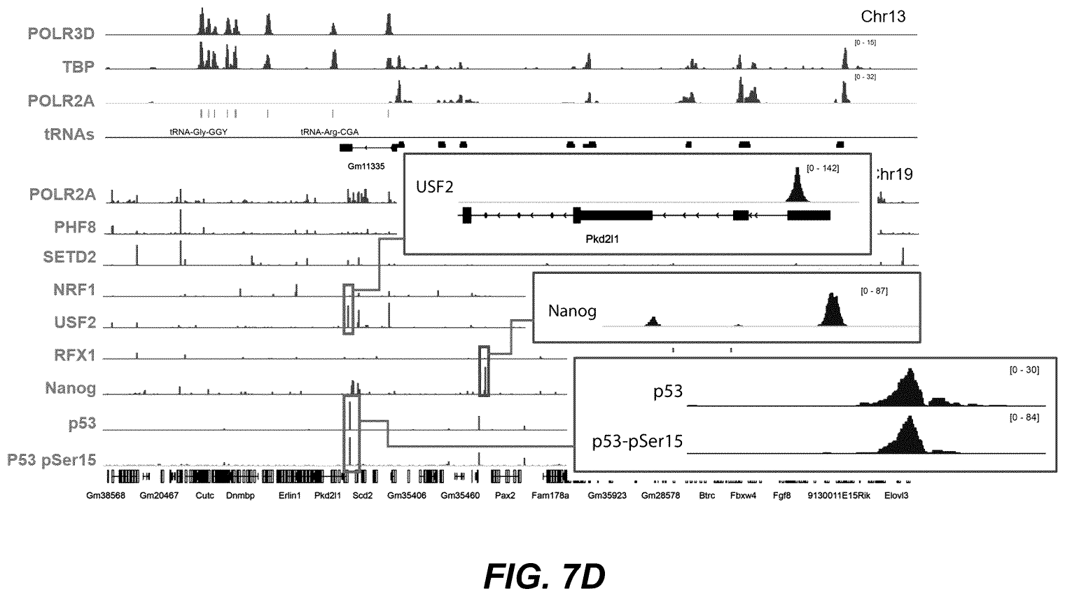

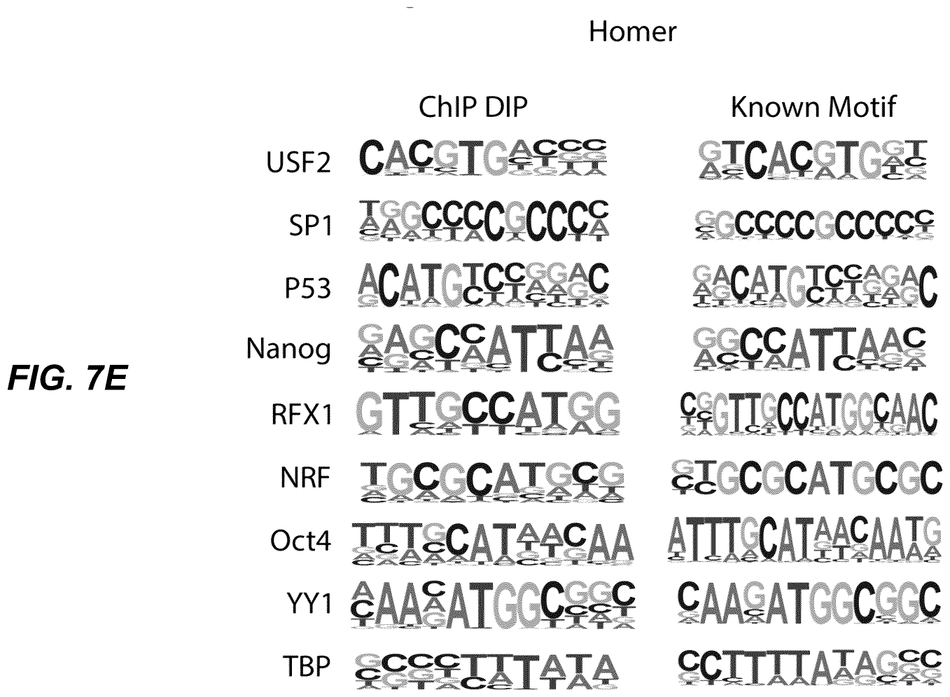

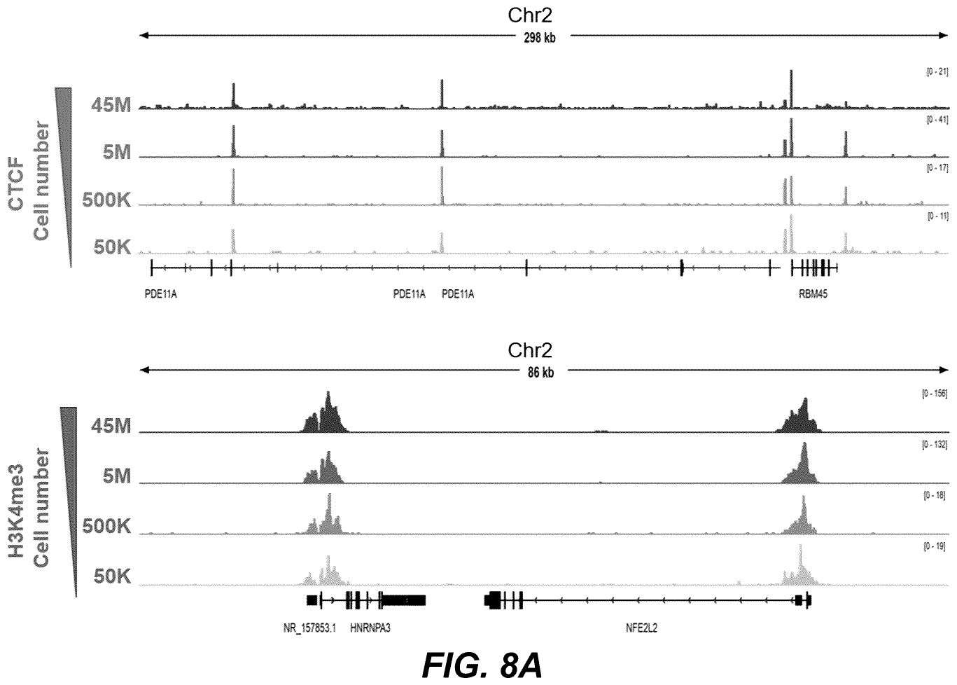

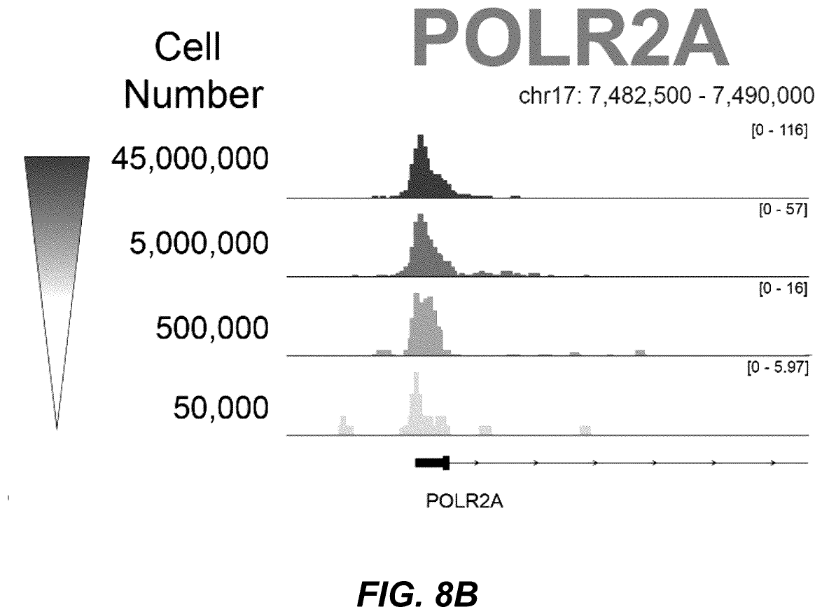

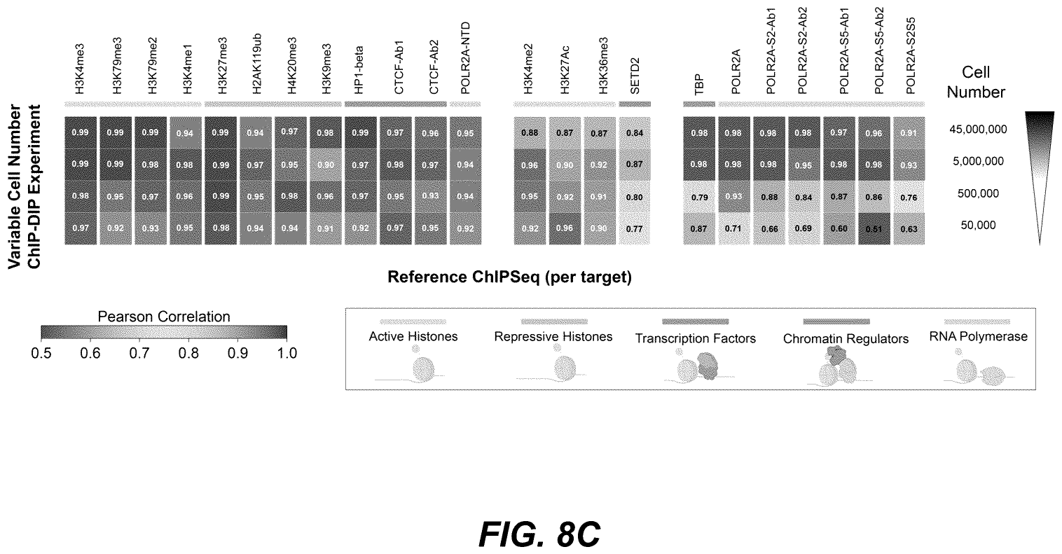

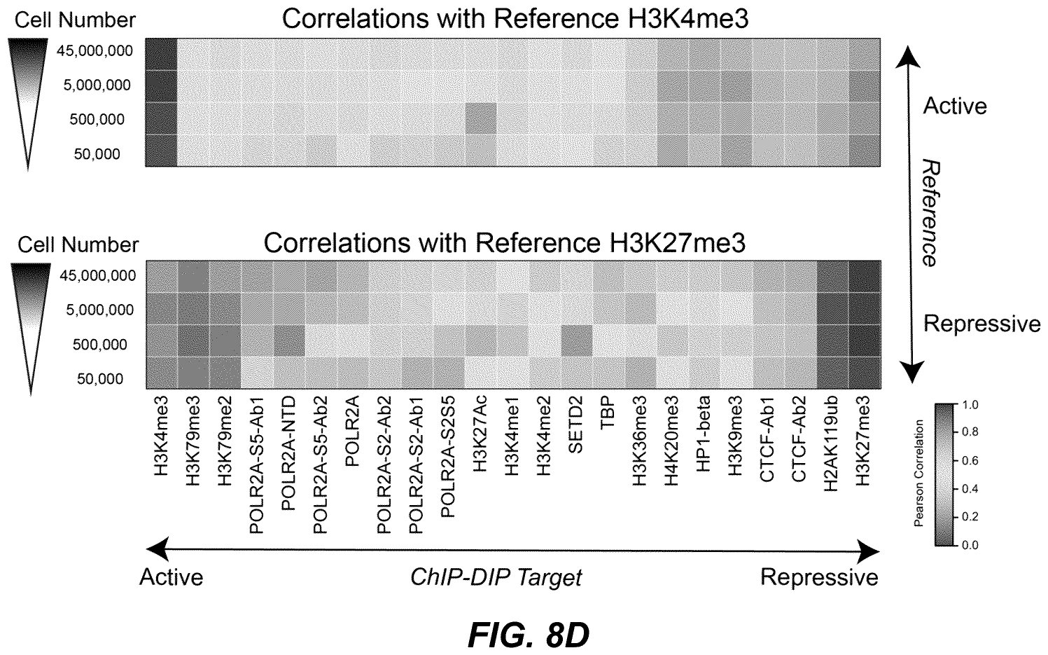

FIGS. 7A-7E depict data related to ChIP-DIP experiments demonstrating the diversity of proteins capable of being handled by the method, including many histone proteins (FIG. 7A (K562)), many chromatin regulators (FIG. 7B (K562) and FIG. 7C (K562)), and many transcription factors (FIG. 7D (mESCs)), as well as data demonstrating that ChIP-DIP data can be used to accurately call motifs (FIG. 7E (mESCs)). In FIG. 7A, the dataset described below in FIG. 8 was visualized on IGV. In FIGS. 7B-C, the same datasets outlined in FIGS. 3A-H were visualized on IGV. For FIG. 7D, a dataset using 169 different antibodies on lysate from mouse embryonic stem cells was generated using the same procedures as above and visualized on IGV. With regards to FIG. 7E, motifs were called using HOMER on a select number of targets from the same dataset as FIG. 7D.

FIGS. 8A-8D depict data related to ChIP-DIP cell type accessibility, including data showing operation of ChIP-DIP with minimal cell numbers (FIG. 8A and FIG. 8B), data showing genome wide concordance as input decreases (FIG. 8C), and data showing Pearson correlations with active or repressive histone modifications for different antibody signals (FIG. 8D) With regards to FIGS. 8A-8D, ChIP DIP was performed on K562 cells using 45,000,000, 5,000,000, 500,000, or 50,000 cells with 35 different antibodies.

The results of this Example provide proof of principle for the use of the ChIP-DIP compositions and methods provided herein for a variety of applications.

### Example 2

### SPIDR

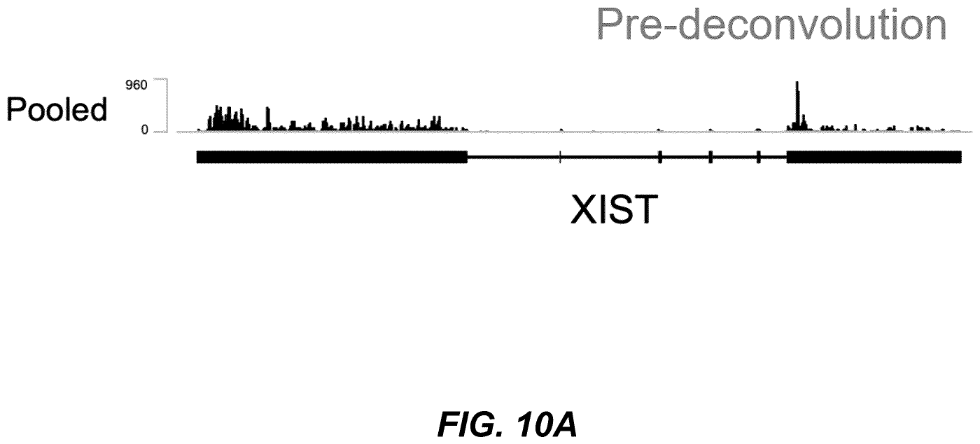

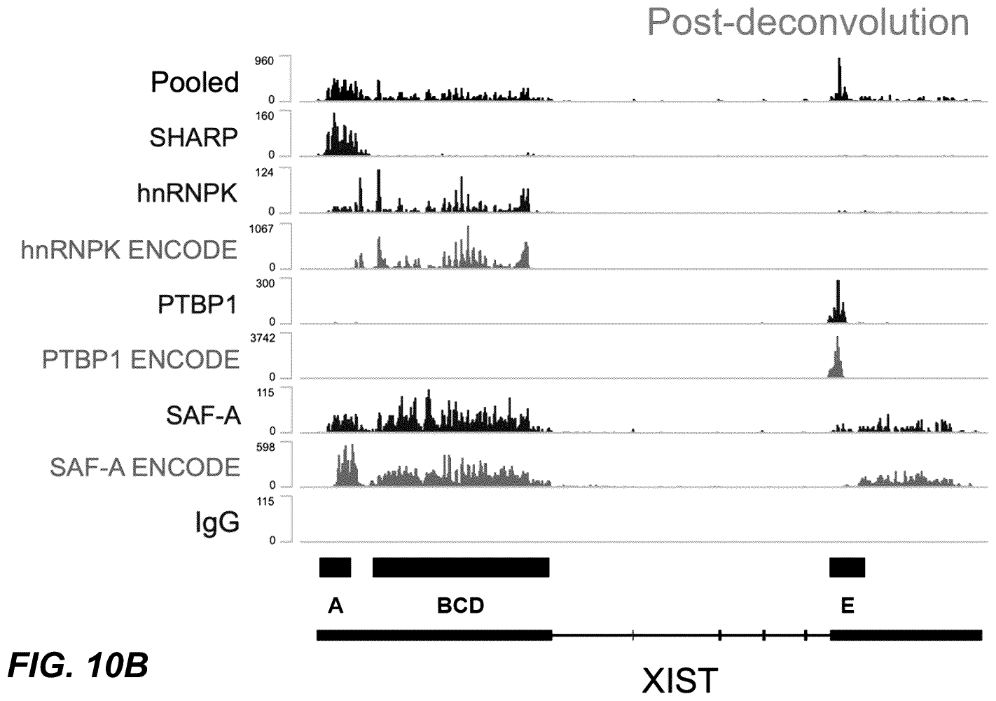

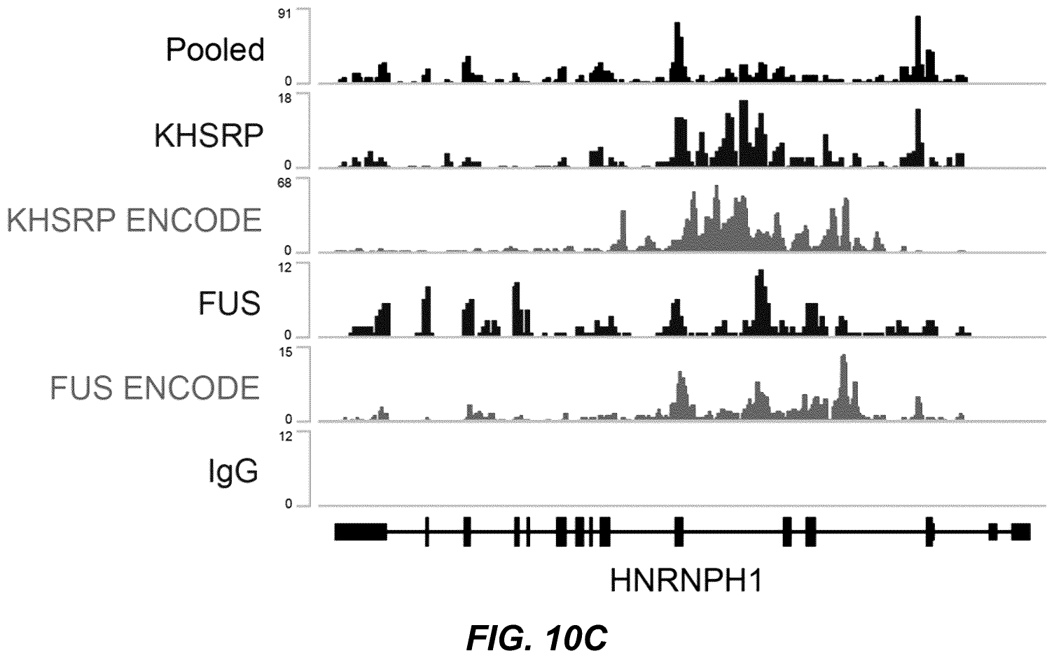

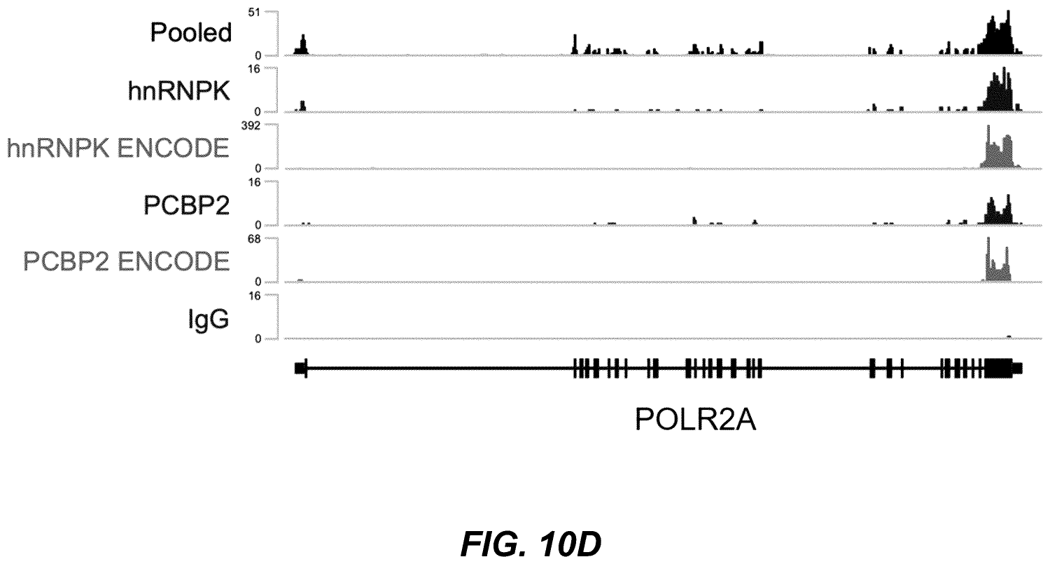

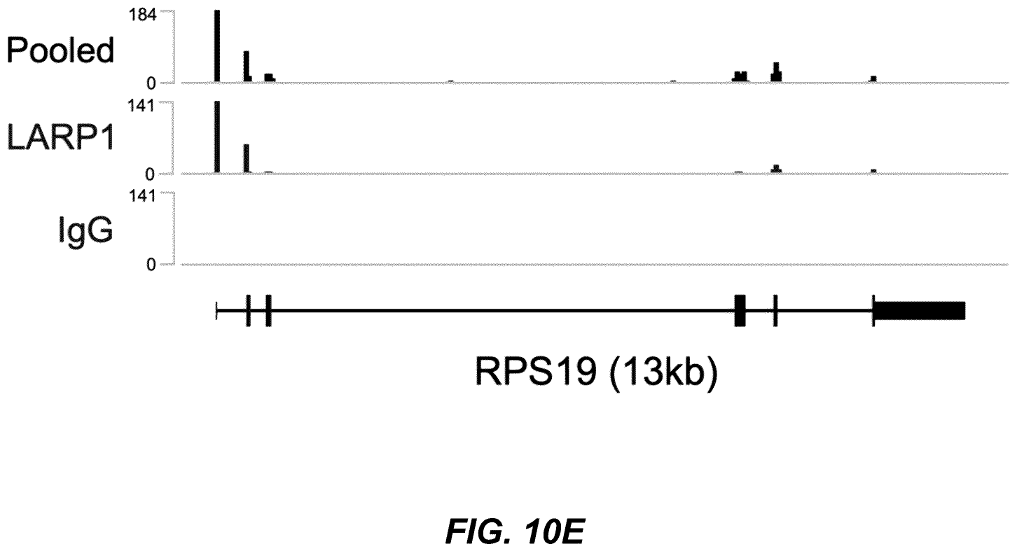

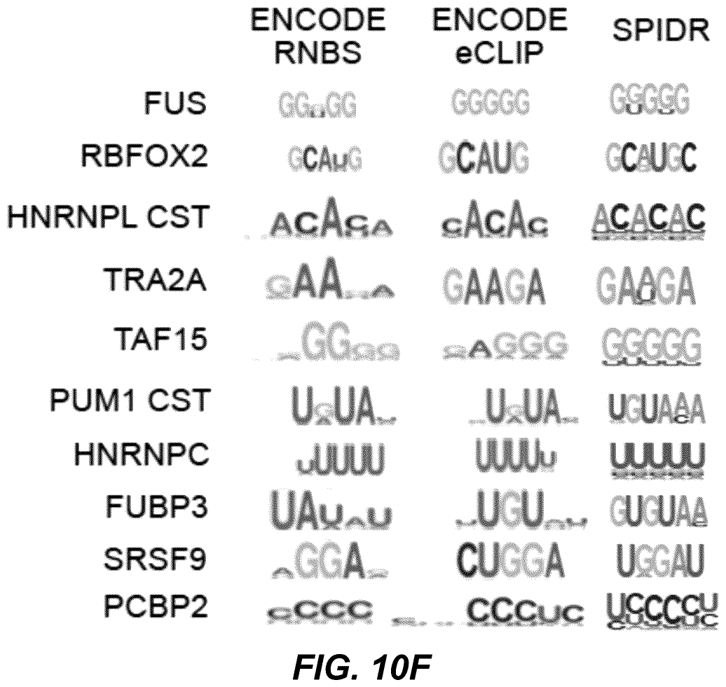

Proof-of-concept SPIDR experiments were conducted versus a variety of RBPs. A non-limiting exemplary workflow for Split Pool Identification of RBPs (SPIDR) is shown in FIG. 9. A set of 25 RBPs that spanned a variety of functions ranging from classic XIST RBPs to splicing proteins to proteins involved in translational regulation were interrogated using SPIDR. The SPIDR experiment was performed on one single sample. Mapped RNA reads for XIST RBPs SHARP, hnRNPK, PTBP1, and SAF-A generated by SPIDR, pre-deconvolution and post-deconvolution, are shown in FIG. 10A and FIG. 10B, respectively. In the pre-deconvolution mapped RNA reads to the transcriptome, as expected, no distinct patterns were observed. Employing the RNA and protein label information from split pool barcoding, one can deconvolute each RNA read and assign it back to its associated protein, and then distinct patterns were observed. These positive results were seen not only for XIST RBPs, but for wide variety of proteins such as splicing and translation where there was a faithful recapitulation of the same binding profile patterns that ENCODE was able to detect. Mapped RNA reads generated by SPIDR for splicing proteins FUS and KHSRP, translation proteins hnRNPK and PCBP2, and translation protein LARP1 are shown in FIGS. 10C-10E, respectively. A comparison of RBP motifs generated by SPIDR, ENCODE RNBS, and ENCODE eCLIP is depicted in FIG. 10F. The results of this Example provide proof of principle for the use of the SPIDR compositions and methods provided herein for a variety of applications.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for detecting interactions between nucleic acid molecules and proteins of interest, comprising:
providing a pool of barcoded detection particles,
wherein each barcoded detection particle comprises a particle associated with an antigen-binding protein and a plurality of barcoding oligonucleotides, wherein the antigen binding protein is capable of specifically binding a protein of interest,
wherein plurality of barcoding, oligonucleotides comprise a first ligand,
wherein the particle comprises a second ligand,
wherein the plurality of barcoding oligonucleotides are associated with the particle via a multivalent binding agent comprising two or more binding moieties capable of binding the first ligand and/or the second ligand,
wherein each barcoding oligonucleotide comprises a capture barcode, wherein the capture barcode is a unique sequence specific to the antigen-binding protein associated with the particle to which the barcoding oligonucleotide is associated with, and
wherein two or more barcoded detection particles of the pool of barcoded detection particles differ from each other with respect to the antigen-binding protein associated with the particle;
lysing a sample comprising a plurality of cells to generate a cell lysate, wherein the cells comprise nucleic acid molecules suspected of being associated with proteins of interest;
contacting the cell lysate, or a product thereof, with the pool of barcoded detection particles to form a plurality of detection complexes, wherein each of the plurality of detection complexes comprises:
a barcoded detection particle;
a captured protein of interest; and
captured nucleic acid molecule(s) associated with the captured protein of interest;
performing two or more iterations of split-and-pool barcoding, wherein each iteration comprises:
(i) randomly distributing the plurality of detection complexes into a plurality of partitions;
(ii) in the plurality of partitions, combinatorially barcoding captured nucleic acid molecules and barcoding oligonucleotides, or products thereof, with a combinatorial barcode unit,
wherein within each partition, the captured nucleic acid molecules and barcoding oligonucleotides are barcoded with the same combinatorial barcode unit,
wherein captured nucleic acid molecules and barcoding oligonucleotides of different partitions receive different combinatorial barcode units from each other, and
wherein captured nucleic acid molecules and barcoding oligonucleotides of the same detection complex will assort together in a partition of the plurality of partitions; and
(iii) pooling the detection complexes from the plurality of partitions,
wherein, after said two or more iterations of split-and-pool barcoding, each combinatorially barcoded captured nucleic acid molecule and each combinatorially barcoded barcoding oligonucleotide comprises a combinatorial barcode comprising two or more combinatorial barcode units, wherein each combinatorial barcode unit corresponds to an iteration of split-and-pool-barcoding;
obtaining sequence information of the combinatorially barcoded captured nucleic acid molecules and the combinatorially barcoded barcoding oligonucleotides, or products thereof; and
detecting interactions between captured nucleic acid molecules and proteins of interest based on the sequence information.

2. The method of claim 1, wherein the pool of barcoded detection particles comprises at least about 2 to about 500 barcoded detection particles that differ from each other with respect to the antigen-binding protein and barcoding oligonucleotide associated with the particle.

3. The method of claim 1, wherein the method further comprises:
(i) adding a crosslinking agent to the plurality of cells prior to the lysis step; or adding a crosslinking agent to the cell lysate;
(ii) isolation of the nuclei of the plurality of cells;
(iii) fragmentation of the chromatin of the plurality of cells, wherein fragmentation comprises enzymatic chromatin fragmentation and/or sonication of the nuclear pellet; and/or
(iv) reversing crosslinking to elute the combinatorially barcoded captured nucleic acid molecules and the combinatorially barcoded barcoding oligonucleotides from the particles.

4. The method of claim 1, wherein the method further comprises:
processing at least one end of the captured nucleic acid molecule(s) to enable ligation of said captured nucleic acid molecule(s) to a ligation adaptor molecule, wherein said processing comprises blunt-ending, phosphorylation, and/or dA-tailing; and/or
ligating a ligation adaptor molecule to the captured nucleic acid molecule(s).

5. The method of claim 1, wherein a probability of the interaction between the nucleic acid molecule and the protein of interest as being bona fide is proportional to the number of iterations of split-and-pool barcoding.

6. The method of claim 1, wherein each combinatorial barcode unit comprises:
at least one 5′ overhang, and wherein said 5′ overhang is capable of ligating to a 5′ overhang of one or more of a ligation adaptor molecule, a combinatorial barcode unit, or a terminal tag; and/or
a modified 5′ phosphate group.

7. The method of claim 1, wherein the combinatorial barcoding step comprises:
annealing the 5′ overhang of a barcoding oligonucleotide, a ligation adaptor molecule, or a combinatorial barcode unit, to the 5′ overhang of a combinatorial barcode unit; and
ligating the annealed molecules.

8. The method of claim 1, wherein the method further comprises, following the two or more iterations of split-and-pool barcoding:
annealing a terminal tag to each captured nucleic acid molecule and each barcoding oligonucleotide; and
ligating said annealed molecules.

9. The method of claim 1, wherein the multivalent binding agent, the first ligand, the second ligand, and/or at least one of the two or more binding moieties is a biotin moiety and/or an avidin moiety.

10. The method of claim 1, wherein obtaining sequence information comprises:
obtaining sequencing data comprising a plurality of sequencing reads of the combinatorially barcoded captured nucleic acid molecules and the combinatorially barcoded barcoding oligonucleotides, or products thereof,
wherein each of the plurality of sequencing reads of the combinatorially barcoded captured nucleic acid molecules, or products thereof, comprise:
a combinatorial barcode sequence, and
a captured nucleic acid molecule sequence; and
wherein each of the plurality of sequencing reads of the combinatorially barcoded barcoding oligonucleotides, or products thereof, comprise:
a combinatorial barcode sequence, and
a capture barcode sequence.

11. The method of claim 1, wherein detecting interactions comprises:
for each unique combinatorial barcode sequence, which indicates a single detection complex of the plurality of detection complexes, identifying the captured nucleic acid molecule sequence and capture barcode sequence of sequencing reads sharing a combinatorial barcode sequence; and/or
for each unique capture barcode sequence, which indicates a captured protein of interest, identifying the captured nucleic acid molecule sequence of sequencing reads sharing a capture barcode sequence.

12. The method of claim 1, wherein the method further comprises:
determining the binding site of a captured protein of interest on associated captured nucleic acid molecule sequence(s); and/or
aligning captured nucleic acid molecule sequence(s) to a reference genome.

13. The method of claim 1, wherein the nucleic acid molecules are selected from the group comprising double-stranded DNA, single-stranded DNA, microRNA (miRNA), messenger RNA (mRNA), long non-coding RNA (lncRNA), ribosomal RNA (rRNA), transfer RNA (tRNA), small nuclear RNA (snRNA), small nucleolar RNA (snoRNA), Piwi-interacting RNA (piRNA), interfering RNA (siRNA), antisense RNA (aRNA), transfer messenger RNA (tmRNA), tRNA-derived small RNA (tsRNA), rDNA-derived small RNA (srRNA), ribozyme, viral RNA, single-stranded RNA, double-stranded RNA, or any combination thereof.

14. The method of claim 1, wherein detecting interactions between nucleic acid molecules and proteins of interest comprises detecting interactions between nucleic acid molecules and at least about 2 to about 500 different proteins of interest.

15. The method of claim 1,
wherein the 5′ end of the barcoding oligonucleotide comprises a modified phosphate group and/or a 5′ overhang capable of ligation to the 5′ overhang of a combinatorial barcode unit;
wherein the barcoding oligonucleotide comprises a unique molecular identifier (HMI);
wherein the barcoding oligonucleotide comprises a universal library sequence, wherein the universal library sequence comprises a sequence complementary to at least a portion of a sequencing primer; and/or
wherein the barcoding oligonucleotide further comprises a 3′ spacer sequence.

16. The method of claim 1, wherein the protein of interest is:
a histone modification selected from the group comprising H2AZK4/K7Ac, H2BK12Ac, H213K15Ac, H2BK20Ac, H3K14Ac, H3K18Ac, H3K9Ac, H3K27Ac, H3K36Ac, H3K56Ac, H3K9/K14Ac, H4K5Ac, H4K12Ac, H4K16Ac, H3Ser10p, H3Thr3p, H2AK119ub, 112AK120ub, H3K4me1, H3K79me1, H3K9me1, H3K27me2, H3K4me2, H3K79me2, H3K9me2, H3K9me2me3, H3K4me3, H3K36me3, H3K36me1, H3K36me2, H3K79me3, H3K9me3, H4K20me3, H3R8me2, H3R3me2, H3R18me2, or any combination thereof; and/or
a chromatin-associated protein selected from the group comprising AEBP2, ATF2, BCL6, Beta Catenin, CBFβ, CDK8 NELFb, CREB, CTCF, DNMT3A, DNMT3B, E2F1, E2F4, EGR1, ELK1, ELL, FoxP1, HIFI, INTS9, KLF5, LAP1α, LAP1β, MAX, MAZ, MBD2, MBD3, MITE, MNT, MeCP2, NRF1, Nanog, Pou5f1, RAD21, RBPJ, RFX1, RNF20, RING1, SP1, SPT16, Suz12, Sox2, TAF1, TBP, TCF4, TET1, TET2, TH1L, USF2, UTX, YY1, ZNF24, ZNF687, cFos, cFos-pSer32, cJun, dun-pSer63, chin-pSer73, P53, P53-pSer15, POLR1A, POLR2A, POLR2A-pSer2, POLR2A-pSer5, POLR2A-pSer2/5, POLR3A, POLR2A-pThr4, POLR3D, POLR3E, ASH2, BAF57, BRD3, BRD4, BRG1, CBP, CLOCK, ESE, EZH2, G9a, HDAC1, HDAC2, HDAC3, HDAC5, HDAC6, HP1α, HP1β, JARID1A, JARID1B, JARID2, JMJD2A, LSD1, MLL, MTA1, MTA2, Menin, NFRkB, PCAF, PHC1, PHF8, RBBP5, RING1B, SAP30, SETD1A, SETD2, S1N3A, SIRT6, SPT4, SPT6, SRC3, SSRP1, WDR5, ZMYND11, or any combination thereof.

17. The method of claim 1, wherein the antigen binding protein:
comprises an antibody, an antibody fragment, an scFv, a Fv, a Fab, a (Fab)2, a single domain antibody (SDAB), a VH or VI, domain, a camelid VIM domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof; and/or
is not conjugated to an oligonucleotide.

18. A method for detecting interactions between ribonucleic acid molecules and RNA-binding proteins (RBPs), comprising:
providing a pool of barcoded detection particles,
wherein each barcoded detection particle comprises a particle associated with an antigen-binding protein and a plurality of barcoding oligonucleotides, wherein the antigen binding protein is capable of specifically binding a REP,
wherein plurality of barcoding oligonucleotides comprise a first ligand,
wherein the particle comprises a second ligand,
wherein the plurality of barcoding oligonucleotides are associated with the particle via a multivalent binding agent comprising two or more binding moieties capable of binding the first ligand and/or the second ligand,
wherein each barcoding oligonucleotide comprises a capture barcode, wherein the capture barcode is a unique sequence specific to the antigen-binding protein associated with the particle to which the barcoding oligonucleotide is associated with, and
wherein two or more barcoded detection particles of the pool of barcoded detection particles differ from each other with respect to the antigen-binding protein associated with the particle;
lysing a sample comprising a plurality of cells to generate a cell lysate, wherein the cells comprise ribonucleic acid molecules suspected of being associated with RBPs;
contacting the cell lysate, or a product thereof, with the pool of barcoded detection particles to form a plurality of detection complexes, wherein each of the plurality of detection complexes comprises:
a barcoded detection particle;
a captured RBP; and
captured ribonucleic acid molecule(s) associated with the captured RBP;
converting the captured ribonucleic acid molecule(s) to complementary DNA (cDNA) molecules;
performing two or more iterations of split-and-pool barcoding, wherein each iteration comprises:
(i) randomly distributing the plurality of detection complexes into a plurality of partitions;
(ii) in the plurality of partitions, combinatorially barcoding cDNA molecules and barcoding oligonucleotides, or products thereof, with a combinatorial barcode unit,
wherein within each partition, the cDNA molecules and barcoding oligonucleotides are barcoded with the same combinatorial barcode unit,
wherein cDNA molecules and barcoding oligonucleotides of different partitions receive different combinatorial barcode units from each other, and
wherein cDNA molecules and barcoding oligonucleotides of the same detection complex will assort together in a partition of the plurality of partitions; and
(iii) pooling the detection complexes from the plurality of partitions,
wherein, after said two or more iterations of split-and-pool barcoding, each combinatorially barcoded cDNA molecule and each combinatorially barcoded barcoding oligonucleotide comprises a combinatorial barcode comprising two or more combinatorial barcode units, wherein each combinatorial barcode unit corresponds to an iteration of split-and-pool-barcoding;
obtaining sequence information of the combinatorially barcoded cDNA molecules and the combinatorially barcoded barcoding oligonucleotides, or products thereof; and
detecting interactions between captured ribonucleic acid molecules and RBPs based on the sequence information.

19. A composition, comprising:
a plurality of barcoded detection particles,
wherein each barcoded detection particle comprises a particle associated with an antigen-binding protein and a plurality of barcoding oligonucleotides,
wherein the antigen-binding protein is associated with the particle via an immunoglobulin-binding moiety,
wherein plurality of barcoding oligonucleotides comprise a first ligand,
wherein the particle comprises a second ligand, and
wherein the plurality of barcoding oligonucleotides are associated with the particle via a multivalent binding agent comprising two or more binding moieties capable of binding the first ligand and/or the second ligand.

20. The composition of claim 19, wherein:
the first ligand comprises biotin;
the second ligand comprises biotin;
the plurality of multivalent binding agents comprise streptavidin;
the particle is a Dynabead; and/or
the immunoglobulin-binding moiety comprises Protein G.
